(19) United States
(12) Patent Application Publication
(10) Pub. No.: US 2018/0256747 A1
(43) Pub. Date: Sep. 13, 2018

(54) COMPOSITIONS AND METHODS RELATED TO SCAVENGER PARTICLES

(71) Applicant: NANOTICS, LLC, Mill Valley, CA (US)

(72) Inventors: Louis Hawthorne, Mill Valley, CA (US); John Dodgson, London (GB)

(21) Appl. No.: 15/738,954

(22) PCT Filed: Jun. 29, 2016
(86) PCT No.: PCT/US2016/040022

§ 371 Date: Dec. 21, 2017

Related U.S. Application Data
(60) Provisional application No. 62/186,838, filed on Jun. 30, 2015.
(60) Provisional application No. 62/198,541, filed on Jul. 29, 2015.
(60) Provisional application No. 62/198,519, filed on Jul. 29, 2015.
(60) Provisional application No. 62/236,507, filed on Oct. 2, 2015.
(60) Provisional application No. 62/319,092, filed on Apr. 6, 2016.

(51) Int. Cl. A61K 47/69; A61K 9/14; A61K 38/17; A61P 35/00; A61P 39/00; A61P 31/18

(52) U.S. Cl. CPC A61K 47/6937; A61K 47/6923; A61K 47/6927; A61K 9/143; A61P 31/18; A61K 38/17; A61P 35/00; A61P 39/00; A61K 9/146

(57) ABSTRACT

The disclosure provides, among other things, compositions that bind to and inhibit the biological activity of soluble biomolecules, as well as pharmaceutical compositions thereof. The compositions may comprise a plurality of particles that specifically bind a target, such as a soluble biomolecule or a biomolecule on the surface of a pathogen, to inhibit the target (or pathogen) from interacting with other molecules or cells. Also provided herein are a number of applications (e.g., therapeutic applications) in which the compositions are useful.

### PRIORITY CLAIM

This application claims priority to U.S. Provisional Patent Application No. 62/186,838, filed Jun. 30, 2015; U.S. Provisional Patent Application No. 62/198,519, filed Jul. 29, 2015; U.S. Provisional Patent Application No. 62/198,541, filed Jul. 29, 2015; U.S. Provisional Patent Application No. 62/236,507, filed Oct. 2, 2015; and U.S. Provisional Patent Application No. 62/319,092, filed Apr. 6, 2016; each of which is hereby incorporated by reference in its entirety.

### BACKGROUND

Dozens of anti-cancer therapies available clinically or under development involve stimulation of the immune system's ability either to recognize or destroy cancer, or both. Three of the most prominent are the anti-checkpoint inhibitors Yervoy® (ipilimumab) from Bristol-Myers Squibb, Keytruda® (pembrolizumab, formerly lambrolizumab) from Merck. However, these and other approaches involve net up-regulation of a subject's immune system, inducing potentially serious symptoms akin to autoimmune disorders and/or other significant side effects.

There is a need in the art for more effective pharmacological approaches for addressing cancer, particularly metastatic cancer, without disturbing a subject's capacity for avoiding auto-immunity. Among other things, the present disclosure provides methods and compositions based on alternative approaches for harnessing a subject's own immune system against cancer, including dis-inhibiting the tumor microenvironment, i.e., weakening the tumor's defensive system, versus stimulating immune cells.

### SUMMARY

The disclosure provides, among other things, compositions that bind to and inhibit the biological activity of biomolecules, especially soluble molecules, as well as pharmaceutical compositions thereof. Also provided herein are a number of applications in which the compositions are useful. For example, compositions described herein are useful for inhibiting proliferation, growth, and/or survival of a cell, such as a cancer cell. Additionally, compositions described herein are useful for preventing and/or treating aging, metabolic disorders, and neurodegenerative diseases. In another example, compositions described herein can be useful to bind to and neutralize toxins (e.g., zootoxins, bacterial toxins, and/or plant toxins), viruses, or other foreign compounds in the circulation of a subject.

### BRIEF DESCRIPTION OF THE DRAWINGS

FIG. 1 depicts an exemplary embodiment of a particle that binds to soluble forms of TNF receptor (sTNF-R). The particle is approximately one cubic micron. The inner surfaces of the particle comprise an immobilized TNF agent, which is capable of binding to the sTNF-R target and sequestering (scavenging) it away from its natural ligands, thereby inhibiting interactions between the sTNF-R target and other proteins and cells. The inner surfaces of the particle define boundaries comprising void space.

FIG. 2 depicts an exemplary embodiment of a particle comprising a TNF agent that binds to soluble forms of a TNF receptor (sTNF-R) target. The three particles shown in FIG. 2 are depicted as having bound 0, 3, or 10 molecules of the sTNF-R target. The ring-shaped particle has a diameter of approximately 175 nm, although the TNF agent and sTNF-R target are not shown to scale. The inner surfaces of the particle contain immobilized TNF agent, which is capable of binding to the sTNF-R target and sequestering (scavenging) it away from its natural ligands, thereby inhibiting interactions between the sTNF-R target and other proteins and cells. The interior of the ring-shaped particle comprises void space.

FIG. 3 depicts exemplary embodiments of particles comprising protrusions. The particle at the left of the figure is an octahedron with a core having a longest dimension of 100 to 150 nm. The particle at the right of the figure is an icosahedron with a core having a longest dimension of 200 to 300 nm. Each particle further comprises molecular protrusions pointing outward from the vertices of the core polyhedral structure. The particles are depicted as comprising an agent, shown in dark gray, and some particles are depicted as having bound a target (e.g., a biomolecule), shown in light gray and identified as 0 or 3 “captures.” The protrusions serve as “cell repellers,” which inhibit interactions between the target bound to the agent of the particle and cell surfaces. The representations of the particles, protrusions, agent, and bound target in FIG. 3 are not necessarily shown to scale.

FIG. 4 consists of two panels, labeled panels (A) and (B). Panel (A) depicts the packing of subparticles within a particle comprising core subparticles and protecting subparticles, wherein each subparticle is substantially spherical and approximately the same size. Nevertheless, a particle may comprise subparticles of varying shapes and/or sizes. Additionally, the subparticles are shown as packing in a hexagonal pattern; however, subparticles may pack randomly or with other geometries. Panel (B) depicts (i) “capture ligands” (i.e., agent), which are immobilized on the surface of core subparticles, (ii) targets (e.g., biomolecules) specifically bound to the agent, and (iii) targets within the fluid-filled void space of the particle. Panel (B) does not depict protecting subparticles. The relative sizes of the subparticles, capture ligands, targets, and void space in FIG. 4 are not necessarily shown to scale.

FIG. 5 consists of four panels, labeled panels (A), (B), (C), and (D). Each panel depicts subparticles of a particle, in which core subparticles are shown in gray and protecting subparticles are shown in white. Each particle comprises 55 core subparticles. Panels (A) and (B) depict views of the particle that are orthogonal to the views depicted in panels (C) and (D). Panels (A) and (C) depict the core subparticles only, and panels (B) and (D) depict the core subparticles and a number of protecting subparticles. A completed particle comprising core subparticles and protecting subparticles is preferably covered by at least one layer of protecting subparticles, which is not shown in its entirety in any panel. In FIG. 5, each core subparticle and protecting subparticle is substantially spherical and approximately the same size; however, the subparticles within a particle may vary in shape and/or size. Additionally, the subparticles of FIG. 5 are shown as packing in a hexagonal pattern; however, the subparticles of a particle may pack with other geometries or they may pack randomly. The relative sizes of the subparticles, capture ligands, targets, and void space in FIG. 5 are not necessarily shown to scale. In particular, the length of the linkers connecting various subparticles may be adjusted to allow for more or less void space between the subparticles.

FIG. 6 consists of 6 panels, labeled panels (A), (B), (C), (D), (E), and (F). Each panel depicts a view of a substantially 2-dimensional particle. In each panel, circles depict agent that is immobilized on the surface of the particle. Substantially 2-dimensional particles may comprise “void space,” e.g., between the arms of a cross or star. Panel (A) depicts a “top-view” of a particle comprising a cross shape, and panel (B) depicts an orthogonal, “side-view” of the same, cross-shaped particle. The “cross shape” of panel (A) is the “substantially 2-dimensional shape,” and the orthogonal, “side-view” is the third dimension, which does not contain the 2-dimensional shape. The “side-view” shows that a substantially 2-dimensional particle may comprise different surfaces, i.e., an “interior surface,” on which the agent is immobilized (black), and an “exterior surface” (i.e., “outer surface”), which is substantially free of agent (gray). The different surfaces may comprise different materials, e.g., the particle may be lamellar, or the different surfaces may be prepared, for example, by masking one surface while the other surface is crosslinked to an agent or a coating molecule. Depending on the size of the particle and the nature of the agent and target, a cross shape will inhibit interactions between a bound target (e.g., biomolecule) and other proteins or cells to varying extents. The geometry of a particle may be adjusted, for example, to further inhibit such interactions. Panel (C) depicts a particle comprising a 6-pointed star geometry, which may inhibit interactions between bound target and other proteins or cells to a greater extent than the cross-shaped particle of panel (A). Panel (D) depicts a 3-pointed star, which may only minimally inhibit interactions between bound target and other proteins or cells. Nevertheless, particles comprising a 3-pointed star geometry may be modified to inhibit interactions between bound target and other proteins or cells to a greater extent. For example, panel (E) depicts a particle comprising a 3-pointed star geometry in which a material that is substantially free of agent encircles the particle, and panel (F) depicts a particle comprising a 3-pointed star geometry (i.e., comprising four 3-pointed stars) having outer surfaces that are substantially free of agent.

### DETAILED DESCRIPTION

The disclosure features compositions and methods for sequestering a soluble biomolecule away from its natural environment, e.g., to thereby inhibit the biological activity of the soluble biomolecule. For example, the disclosure provides a particle, or a plurality of particles, having a surface comprising an agent (e.g., immobilized on a surface of the particle) that selectively binds to a soluble biomolecule. Once the soluble biomolecule is bound by the agent, it is sequestered by the particle such that the soluble biomolecule has a reduced ability (e.g., substantially reduced ability or no ability) to interact with other natural binding partners of the soluble biomolecule. Thus, the soluble biomolecule becomes inert.

### I. Biomolecule

The soluble biomolecule is, generally, a first member of a specific binding pair. As used herein, a “binding partner,” “specific binding partner,” or a “member of a specific binding pair,” generally comprises any member of a pair of binding members that bind to each other with substantial affinity and specificity. A pair of binding partners may bind to one another to the substantial exclusion of at least most or at least substantially all other components of a sample, and/or may have a dissociation constant of less than about 10⁻⁴, 10⁻⁵, 10⁻⁶, 10⁻⁷, or 10⁻⁸M, among others. A pair of binding partners may “fit” together in a predefined manner that relies on a plurality of atomic interactions to cooperatively increase specificity and affinity. Binding partners may be derived from biological systems (e.g., receptor-ligand interactions), chemical interactions, and/or by molecular imprinting technology, among others. Exemplary corresponding pairs of binding partners, also termed specific binding pairs, are presented in Table 1, with the designations “first” and “second” being arbitrary and interchangeable.

The term “biomolecule” as used herein, refers to any molecule that may exert an effect on a living organism. In some embodiments, the biomolecule is an atom, such as lithium or lead (e.g., the biomolecule may be a metal cation). In some embodiments, the biomolecule is not an atom or metal ion. For example, the biomolecule may be a molecule, such as an organic compound or inorganic compound. In some embodiments, the biomolecule is a drug, such as warfarin or dabigatran. The biomolecule may be a psychoactive drug, such as diacetylmorphine. The biomolecule may be a poison, toxin, or venom. The biomolecule may be an allergen. The biomolecule may be a carcinogen. The biomolecule may be the agent of a chemical weapon, such as a nerve agent. The biomolecule may be a molecule that is endogenous to the organism, such as a hormone, cytokine, neurotransmitter, soluble extracellular receptor, antibody, or soluble matrix protein. The biomolecule may be a peptide, polypeptide, protein, nucleic acid, carbohydrate, or sugar. The biomolecule may comprise a peptide, polypeptide, protein, nucleic acid, carbohydrate, or sugar. The biomolecule may be a misfolded protein. The biomolecule may be an amyloid or the soluble precursor of an amyloid. “Polypeptide,” “peptide,” and “protein” are used interchangeably and mean any peptide-linked chain of amino acids, regardless of length or post-translational modification. The biomolecule may be a lipid, a steroid, or cholesterol. The biomolecule may comprise a lipid, a steroid, or cholesterol. The biomolecule may be a circulating, cell-free nucleic acid, such as a circulating, cell-free RNA. The biomolecule may be a micro RNA (miRNA).

The biomolecule may be a biomolecule that is secreted by a cell (e.g., a mammalian cell). The biomolecule may be an extracellular region of a membrane protein that is susceptible to cleavage into a soluble form. The biomolecule may be a cytosolic biomolecule. For example, the biomolecule may be a cytosolic biomolecule that is released in vivo following apoptosis, or a particle may be used in an in vitro method in which the cytosolic biomolecule is free in solution.

In certain preferred embodiments, the biomolecule is a soluble biomolecule. In certain preferred embodiments, the target is a soluble biomolecule. Nevertheless, a particle may target biomolecules that are not solutes in aqueous solution, and/or that do not interact with binding partners on a cell surface. For example, a particle may specifically bind a biomolecule that is associated with a protein aggregate, such as amyloid or a prion aggregate. Such particles may provide a therapeutic benefit by disassembling the aggregate (e.g., by shifting a thermodynamic equilibrium away from aggregated states) and/or by sequestering the aggregate (e.g., to inhibit further aggregation and/or to allow for clearance of the bound aggregate). Similarly, a particle may specifically bind to crystalline calcium or hydroxyapatite. Similarly, a particle may specifically bind to a biomolecule that is associated with a virus or cell, such as a bacterial, protozoan, fungal, or yeast cell, e.g., wherein the biomolecule is not a solute in aqueous solution, but the biomolecule is partitioned into a membrane, cell wall, or capsid. Thus, a particle may sequester a pathogenic virus or cell, thereby attenuating the pathogenicity of the virus or cell. A particle may specifically bind to a biomolecule that is associated with an extracellular vesicle, such as an ectosome, exosome, shedding vesicle, or apoptotic body. A particle may specifically bind to a low-density lipoprotein, e.g., to sequester low-density lipoprotein particles.

The biomolecule may be a ligand of a cell surface receptor. The ligand may be a naturally-occurring ligand or a synthetic ligand. The ligand may be a native ligand of the receptor (e.g., a ligand that is produced by a subject in vivo) or a non-native ligand (e.g., a ligand that is introduced into the subject, such as a virus or drug). The biomolecule may be a ligand for a cytosolic receptor or a nuclear receptor.

TABLE 1
Examples of specific binding pairs.

| First Binding Partner | Second Binding Partner |
|---|---|
| Cell Surface Receptor | Natural Ligand |
| (e.g., TNF receptor) | (e.g., TNFα) |
| Viral Coat or Envelope Protein | Corresponding Cellular |
| (e.g., HIV-1 gp120) | Receptor (e.g., CD4) |
| Botulinum Toxin | Synaptotagmin II Cell |
| | Surface Receptor |
| Soluble Receptor (e.g., soluble | Natural Ligand |
| TNFR or soluble IL-2 receptor) | (e.g., TNFα or IL-2) |

Tumor cells are known to protect themselves from host immune surveillance by shedding soluble forms of cytokine receptors, which soluble receptors bind to the cytokines produced by immune cells in the tumor microenvironment. For example, cancer cells shed soluble forms of TNF receptor and other cytokine receptors, such as IL-2 receptor and TRAIL receptor. These soluble receptors confer a growth advantage to cancer cells by relieving the cells of the pro-apoptotic effects of the TNFα, IL-2, and TRAIL. Karpatova et al. report the shedding of the 67 kD laminin receptor by human cancer cells, which may augment tumor invasion and metastasis (J Cell Biochem 60(2):226-234 (1996)). Thus, the particles described herein can be engineered for scavenging soluble forms of cell surface receptor proteins, e.g., for use in the treatment of cancer.

Accordingly, in some embodiments, the cell surface receptor protein is expressed by a cancer cell and/or the cell surface receptor protein is a protein shed by the cancer cell as a soluble form of the cell surface receptor protein. In some embodiments, the cell surface receptor protein, when activated, induces apoptosis (e.g., a death receptor). In some embodiments, the cell surface receptor protein is a tumor necrosis factor receptor (TNFR) protein (e.g., TNFR-1 or TNFR-2). In some embodiments, the cell surface receptor protein is a Fas receptor protein. In some embodiments, the cell surface receptor protein is a TNF-related apoptosis-inducing ligand receptor (TRAILR) protein, 4-1BB receptor protein, CD30 protein, EDA receptor protein, HVEM protein, lymphotoxin beta receptor protein, DR3 protein, or TWEAK receptor protein. In some embodiments, the cell surface receptor protein is an interleukin receptor protein, e.g., an IL-2 receptor protein. It is understood that in such embodiments, the target soluble biomolecule can be a soluble form of the cell surface receptor, e.g., shed from a cancer cell.

In some embodiments, the biomolecule is soluble Tim3 (“T-Cell Ig Mucin 3”). Soluble Tim3 (sTim3) has been implicated in autoimmune disease and cancer, and elevated sTim3 is associated with HIV infection. The association of Galectin 9 (“Gal9”) and potentially other ligands with Tim3 in heterodimeric association with CEACAM1 leads to inhibition of T-cell responses, and co-blockade of Tim3 and CEACAM1 leads to anti-tumor immune response. Accordingly, the biomolecule may be sTim3 or a natural ligand for sTim3, such as Tim3L, or Ga19. A biomolecule may be a soluble isoform of CEACAM1. In this way, the particles may be adapted to scavenge sTim3 while not inhibiting interaction between Gal9 and membrane-bound Tim3 (mTim3). Similarly an agent may be sTim3, an antibody selective for sTim3 (or an antigen binding portion thereof), or a ligand for Tim3. An agent may be a natural ligand for CEACAM1 (such as Gal9 or variant thereof) or an antibody selective for either CEACAM1 or its soluble isoform. Any of the foregoing particles may be used, for example, in methods of treating cancer, methods of treating HIV infection, and methods of treating an autoimmune disease, such as graft-versus-host disease.

In some embodiments, the biomolecule may be Gal9 (Galectin 9). A particle may comprise an agent selective for Ga19, such as a natural ligand for Ga19, such as Tim3, or a variant thereof, or an antibody selective for Ga19. In this way, the particles may be adapted to scavenge Gal9 while not inhibiting interactions of membrane-bound Gal9 (mGa19) with membrane-bound Tim3 (mTim3). In some embodiments, the biomolecule may be a soluble isoform of CEACAM1 (“sCEACAM1”). An agent may be a natural ligand for sCEACAM1, such as Ga19, or a variant thereof, or an antibody selective for either CEACAM1 or a soluble isoform of CEACAM1.

In some embodiments, the biomolecule is soluble CTLA4. Soluble CTLA4 (“sCTLA4”) has been implicated in cancer, and antibodies active against sCTLA4, but not against membrane bound CTLA4 (“mCTLA4”), are efficacious in animal models of cancer. In some embodiments, the biomolecule is sCTLA4. An agent may be a natural ligand for CTLA4, such as soluble B7-1 or soluble B7-2, or a variant thereof, or an antibody selective for CTLA4, such as ipilimumab or ticilimumab. In this way, particles may be adapted to scavenge sCTLA4 without inhibiting interaction between ligands and mCTLA4. Thus, sCTLA4 may be removed from the tumor microenvironment (“TME”) and/or the circulation outside of the TME while leaving mCTLA4 free for interaction as part of a normal immune response. Particles that target sCTLA4 may be used, for example, in methods of treating with cancer.

Soluble PD-1 (“sPD1”) is implicated in autoimmune diseases such as rheumatoid arthritis. Excess sPD1 may disturb the balance between PD1 and its ligands PD-L1 and PD-L2, leading to autoimmunity. Thus, the biomolecule may be sPD1. An agent may be a natural ligand for sPD1, such as PD-L1, PD-L2, or a variant thereof, or an antibody selective for PD1, such as a PD1 blockade drug, for example, nivolumab, pidilizumab, or pembrolizumab (Keytruda®). Thus, a particle may be adapted to scavenge sPD1 without inhibiting an interaction of PD-L1 or PD-L2 with membrane-bound PD1. Such particles may be used, for example, in methods of treating autoimmune diseases, such as arthritis.

LAG3 is a T-cell surface receptor that, when bound by its ligand, results in inhibition. Soluble forms of LAG3 (“sLAG3”) correlate with autoimmunity, for example, in Type I diabetes and in other autoimmune diseases. The biomolecule may be sLAG3. An agent may be a natural ligand for sLAG3, or a variant thereof, or an antibody selective for sLAG3. Thus, a particle may adapted to scavenge sLAG3 without inhibiting interactions between ligands and membrane-bound LAG3. Such particles may be used, for example, in methods of treating an autoimmune disease, such as type I diabetes.

The biomolecule may be TNFα. The agent may comprise an anti-TNFα antibody, such as infliximab, adalimumab, cerolizumab, afelimomab, nerelimomab, ozoralizumab, or golimumab, or an the agent may comprise the antigen-binding portion of an anti-TNFα antibody. The agent may be etanercept. The agent may be a soluble receptor for TNFα (sTNF-R or a variant thereof). Particles targeting TNFα may be particularly useful for treating or preventing various autoimmune diseases, such as ankylosing spondylitis, Crohn's disease, hidradenitis suppurativa, psoriasis, plaque psoriasis, psoriatic arthritis, refractory asthma, juvenile idiopathic arthritis, ulcerative colitis, and rheumatoid arthritis. Particles targeting TNFα may also be useful for treating or preventing Alzheimer's disease, cardiovascular disease, type II diabetes, muscular dystrophy, and obesity, in addition to other diseases and conditions.

The biomolecule may be β2 microglobulin (B2M). The agent may be an anti-B2M antibody. Particles targeting B2M may be useful for treating or preventing memory loss, cognitive decline, peripheral arterial disease, dialysis-related amyloidosis, chronic lymphocytic leukaemia, multiple myeloma, and lymphoma, in addition to other diseases and conditions.

The biomolecule may be CCL2 (chemokine (C-C motif) ligand 2). The agent may be an anti-CCL2 antibody. Particles targeting CCL2 may be useful for treating or preventing Alzheimer's disease, atherosclerosis, ischemia (e.g., ischemic stroke), epilepsy, multiple sclerosis, psoriasis, rheumatoid arthritis, glomerulonephritis, and traumatic brain injury, in addition to other diseases and conditions.

The biomolecule may be CCL11 (C-C motif chemokine 11; eotaxin 1). The agent may be an anti-CCL11 antibody. Particles targeting CCL11 may be useful for treating or preventing memory loss and cognitive decline, in addition to other diseases and conditions.

The biomolecule may be CCL19. The agent may be an anti-CCL19 antibody. Particles targeting either CCL19 may be useful for treating or preventing aging and cognitive decline, in addition to other diseases and conditions.

The biomolecule may be interferon gamma (INFγ). The agent may comprise an anti-INFγ antibody, such as fontolizumab, or a soluble INFγ receptor (sINFγR). The biomolecule may be soluble INFγ receptor. The agent may comprise INFγ or an anti sINFγR antibody. Particles targeting interferon gamma may be particularly useful for treating or preventing autoimmune disease, such as Crohn's disease, rheumatoid arthritis, and psoriasis, in addition to other diseases and conditions.

The biomolecule may be clusterin (e.g., secretory clusterin, isoform 2). The agent may comprise an anti-clusterin antibody, or an antigen-binding portion thereof. Particles targeting clusterin may be useful for treating or preventing cancer (e.g., head and neck cancer, renal cell cancer, colorectal cancer, endometrial cancer, ovarian cancer, breast cancer, prostate cancer, pancreatic cancer, lung cancer, hepatocellular cancer, or melanoma), renal disease (e.g., nephropathic cystinosis), Fanconi syndrome, glomerulonephritis, atherosclerosis, and myocardial infarction, in addition to other diseases and conditions.

The biomolecule may be high mobility group box 1 (HMGB1). The agent may comprise an anti-HMGB1 antibody, or an antigen-binding portion thereof. The biomolecule may be a heat shock protein (e.g., HSP60, HSP70, HSP90). The agent may comprise an anti-HSP antibody, or an antigen-binding portion thereof. The biomolecule may be a peroxiredoxin (e.g., peroxiredoxin 1 or peroxiredoxin 2). The agent may comprise an anti-peroxiredoxin antibody, or an antigen-binding portion thereof.

The agent may be the extracellular portion of a scavenger receptor, such as a class A scavenger receptor (e.g., SCARA1 (Macrophage scavenger receptor 1; MSR1; CD204), SCARA2 (Macrophage receptor; MARCO), SCARA3, SCARA4 (COLEC12), SCARA5), class B scavenger receptor (e.g., SCARB1, SCARB2, SCARB3 (CD36)), CD68, mucin, or lectin-like oxidized LDL receptor-1 (LOX-1).

The biomolecule may be insulin-like growth factor 1 (IGF-1) or an insulin-like growth factor binding protein (e.g., IGFBP-1, IGFBP-2, IGFBP-3, IGFBP-4, IGFBP-5, IGFBP-6). The agent may be insulin-like growth factor 1 (IGF-1) or an insulin-like growth factor binding protein (e.g., IGFBP-1, IGFBP-2, IGFBP-3, IGFBP-4, IGFBP-5, IGFBP-6). The agent may be an antibody, or an antigen-binding portion thereof, that selectively binds insulin-like growth factor 1 (IGF-1) or an insulin-like growth factor binding protein (e.g., IGFBP-1, IGFBP-2, IGFBP-3, IGFBP-4, IGFBP-5, IGFBP-6).

The agent may be an antibody that selectively binds an extracellular epitope of CD63, CD9, or CD81. Particles targeting CD63, CD9, and/or CD81 may be particularly useful for scavenging extracellular vesicles, such as an ectosome, exosome, shedding vesicle, or apoptotic body. Particles that scavenge various extracellular vesicles may be particularly useful for treating or preventing cancer (e.g., cancers having a disease progression that correlates with the shedding of vesicles).

The biomolecule may be CXCL1, CXCL2, CXCL3, CXCL4, CXCL4L1, CXCL5, CXCL6, CXCL7, CXCL8, CXCL9, CXCL10, CXCL11, CXCL12, CXCL13, CXCL14, CXCL16, CXCL17, CCL1, CCL2, CCL3, CCL3L1, CCL3L3, CCL4, CCL4L1, CCL4L2, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, XCL1, XCL2, or CX3CL1 (see, e.g., Zlotnik, A. and Yoshie, O., Immunity, 36(5):705 (2012)). The agent may comprise an antibody (or an antigen-binding portion thereof) that specifically binds CXCL1, CXCL2, CXCL3, CXCL4, CXCL4L1, CXCL5, CXCL6, CXCL7, CXCL8, CXCL9, CXCL10, CXCL11, CXCL12, CXCL13, CXCL14, CXCL16, CXCL17, CCL1, CCL2, CCL3, CCL3L1, CCL3L3, CCL4, CCL4L1, CCL4L2, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, XCL1, XCL2, or CX3CL1.

The biomolecule may be interleukin 1, interleukin 1 alpha, interleukin 1 beta, interleukin 2, interleukin 3, interleukin 4, interleukin 5, interleukin 6, interleukin 7, interleukin 8, interleukin 9, interleukin 10, interleukin 11, interleukin 12, interleukin 13, interleukin 14, interleukin 15, interleukin 16, interleukin 17, interleukin 18, interleukin 19, interleukin 20, interleukin 21, interleukin 22, interleukin 23, interleukin 24, interleukin 25, interleukin 26, interleukin 27, interleukin 28, interleukin 29, interleukin 30, interleukin 31, interleukin 32, interleukin 33, interleukin 35, or interleukin 36. The agent may comprise an antibody (or an antigen-binding portion thereof) that specifically binds interleukin 1, interleukin 1 alpha, interleukin 1 beta, interleukin 2, interleukin 3, interleukin 4, interleukin 5, interleukin 6, interleukin 7, interleukin 8, interleukin 9, interleukin 10, interleukin 11, interleukin 12, interleukin 13, interleukin 14, interleukin 15, interleukin 16, interleukin 17, interleukin 18, interleukin 19, interleukin 20, interleukin 21, interleukin 22, interleukin 23, interleukin 24, interleukin 25, interleukin 26, interleukin 27, interleukin 28, interleukin 29, interleukin 30, interleukin 31, interleukin 32, interleukin 33, interleukin 35, or interleukin 36. The agent may comprise a soluble interleukin-2 receptor, soluble interleukin-3 receptor, soluble interleukin-4 receptor, soluble interleukin-5 receptor, soluble interleukin-6 receptor, soluble interleukin-7 receptor, soluble interleukin-9 receptor, soluble interleukin-10 receptor, soluble interleukin-11 receptor, soluble interleukin-12 receptor, soluble interleukin-13 receptor, soluble interleukin-15 receptor, soluble interleukin-20 receptor, soluble interleukin-21 receptor, soluble interleukin-22 receptor, soluble interleukin-23 receptor, soluble interleukin-27 receptor, or soluble interleukin-28 receptor. The agent may be soluble ST2, which binds interleukin 33.

The biomolecule may be a soluble interleukin-2 receptor, soluble interleukin-3 receptor, soluble interleukin-4 receptor, soluble interleukin-5 receptor, soluble interleukin-6 receptor, soluble interleukin-7 receptor, soluble interleukin-9 receptor, soluble interleukin-10 receptor, soluble interleukin-11 receptor, soluble interleukin-12 receptor, soluble interleukin-13 receptor, soluble interleukin-15 receptor, soluble interleukin-20 receptor, soluble interleukin-21 receptor, soluble interleukin-22 receptor, soluble interleukin-23 receptor, soluble interleukin-27 receptor, or soluble interleukin-28 receptor. The agent may comprise an antibody (or an antigen-binding portion thereof) that specifically binds soluble interleukin-2 receptor, soluble interleukin-3 receptor, soluble interleukin-4 receptor, soluble interleukin-5 receptor, soluble interleukin-6 receptor, soluble interleukin-7 receptor, soluble interleukin-9 receptor, soluble interleukin-10 receptor, soluble interleukin-11 receptor, soluble interleukin-12 receptor, soluble interleukin-13 receptor, soluble interleukin-15 receptor, soluble interleukin-20 receptor, soluble interleukin-21 receptor, soluble interleukin-22 receptor, soluble interleukin-23 receptor, soluble interleukin-27 receptor, or soluble interleukin-28 receptor. The agent may be interleukin 2, interleukin 3, interleukin 4, interleukin 5, interleukin 6, interleukin 7, interleukin 9, interleukin 10, interleukin 11, interleukin 12, interleukin 13, interleukin 15, interleukin 20, interleukin 21, interleukin 22, interleukin 23, interleukin 27, or interleukin 28.

The biomolecule may be epinephrine, norepinephrine, melatonin, serotonin, triiodothyronine, or thyroxine. The biomolecule may be a prostaglandin (e.g., prostacyclin 12 (PGI2), prostaglandin E2 (PGE2), prostaglandin F2α (PGF2α)), a leukotriene, prostacyclin, or thromboxane. The biomolecule may be testosterone, dehydroepiandrosterone (DHEA), androstenedione, dihydrotestosterone (DHT), aldosterone, estrone, estradiol, estriol, progesterone, cortisol, calcitriol, or calcidiol.

The biomolecule may be amylin, adiponectin, adrenocorticotropic hormone, angiotensinogen, angiotensin I, angiotensin II, antidiuretic hormone (vasopressin), apelin, atrial-natriuretic peptide, brain natriuretic peptide, calcitonin, chemerin, cholecystokinin, corticotropin-releasing hormone, cortistatin, enkephalin, endothelin, erythropoietin, follicle-stimulating hormone, galanin, gastric inhibitory polypeptide, gastrin, ghrelin, glucagon, glucagon-like peptide-1, gonadotropin-releasing hormone, growth hormone-releasing hormone, hepcidin, human chorionic gonadotropin, human placental lactogen, growth hormone, inhibin, insulin, insulin-like growth factor (somatomedin, e.g., IGF-I), leptin, lipotropin, luteinizing hormone, melanocyte stimulating hormone, motilin, orexin, oxytocin, pancreatic polypeptide, parathyroid hormone, pituitary adenylate cyclase-activating peptide, prolactin, prolactin releasing hormone, relaxin, renin, secretin, somatostatin, thrombopoietin, thyroid-stimulating hormone (thyrotropin), thyrotropin-releasing hormone, or vasoactive intestinal peptide. The agent may comprise an antibody (or an antigen-binding portion thereof) that specifically binds amylin, adiponectin, adrenocorticotropic hormone, apelin, angiotensinogen, angiotensin I, angiotensin II, antidiuretic hormone (vasopressin), atrial-natriuretic peptide, brain natriuretic peptide, calcitonin, chemerin, cholecystokinin, corticotropin-releasing hormone, cortistatin, enkephalin, endothelin, erythropoietin, follicle-stimulating hormone, galanin, gastric inhibitory polypeptide, gastrin, ghrelin, glucagon, glucagon-like peptide-1, gonadotropin-releasing hormone, growth hormone-releasing hormone, hepcidin, human chorionic gonadotropin, human placental lactogen, growth hormone, inhibin, insulin, insulin-like growth factor (somatomedin, e.g., IGF-I), leptin, lipotropin, luteinizing hormone, melanocyte stimulating hormone, motilin, orexin, oxytocin, pancreatic polypeptide, parathyroid hormone, pituitary adenylate cyclase-activating peptide, prolactin, prolactin releasing hormone, relaxin, renin, secretin, somatostatin, thrombopoietin, thyroid-stimulating hormone (thyrotropin), thyrotropin-releasing hormone, or vasoactive intestinal peptide.

The biomolecule may be vascular endothelial growth factor-A (VEGF-A). The agent may comprise an antibody that specifically binds VEGF-A, such as bevacizumab or brolucizumab, or an antigen-binding portion thereof, such as ranibizumab. For example, the agent may be aflibercept. Particles that target VEGF-A may be particularly useful for treating or preventing macular degeneration (e.g., wet macular degeneration), proliferative diabetic retinopathy, neovascular glaucoma, macular edema, cancer (e.g., colorectal cancer, lung cancer, prostate cancer, breast cancer, renal cancer, brain cancer), bronchial asthma, diabetes mellitus, ischemic cardiomyopathy, and myocardial ischemia, in addition to other conditions and diseases.

The biomolecule may be a soluble vascular endothelial growth factor receptor, such as soluble vascular endothelial growth factor receptor 1 (soluble VEGFR-1), soluble vascular endothelial growth factor receptor 2 (soluble VEGFR-2), or soluble vascular endothelial growth factor receptor 3 (soluble VEGFR-3). The agent may be an antibody, or antigen-binding portion thereof, that selectively binds a soluble VEGF receptor, such as alacizumab, icrucumab, or ramucirumab. The agent may be a ligand of a VEGF receptor, such as VEGF-A, VEGF-B, VEGF-C, VEGF-D, or placental growth factor (PGF). Particles targeting soluble VEGF receptors may be particularly useful for treating or preventing cancer, in addition to other disease and conditions.

The biomolecule may be a member of the epidermal growth factor family, such as epidermal growth factor (EGF), heparin-binding EGF-like growth factor (HB-EGF), transforming growth factor-α (TGF-α), amphiregulin (AR), epiregulin (EPR), epigen, betacellulin (BTC), neuregulin-1 (NRG1), neuregulin-2 (NRG2), neuregulin-3 (NRG3), or neuregulin-4 (NRG4). The agent may be an antibody, or antigen-binding portion thereof, that selectively binds EGF, HB-EGF, TGF-α, AR, EPR, epigen, BTC, NRG1, NRG2, NRG3, or NRG4. The agent may comprise a soluble EGF receptor, such as soluble EGF receptor, soluble HER2, or soluble HER3. Particles targeting members of the epidermal growth factor family may be particularly useful for treating or preventing cancer, in addition to other conditions and diseases.

The biomolecule may be a soluble epidermal growth factor receptor (EGF receptor), such as soluble EGF receptor, soluble human epidermal growth factor receptor 2 (soluble HER2) or soluble human epidermal growth factor receptor 3 (soluble HER3). The agent may be an antibody, or antigen-binding portion thereof, that selectively binds a soluble EGF receptor, such as cetuximab, futuximab, imgatuzumab, matuzumab, necitumumab, nimotuzumab, panitumumab, zalutumumab, duligotumab, patritumab, ertumaxomab, pertuzumab, or trastuzumab. The agent may be a ligand of an EGF receptor, such as an EGF family member as described above. Particles targeting soluble EGF receptors may be particularly useful for treating or preventing cancer, in addition to other disease and conditions.

The biomolecule may be an IgE antibody. The agent may comprise an anti-IgE antibody, such as omalizumab or talizumab, or an antigen-binding portion thereof. The agent may be the extracellular portion of FcεRI. Particles that target IgE antibodies may be particularly useful for treating chronic spontaneous urticarial and allergic asthma, in addition to other conditions and diseases.

The biomolecule may be proprotein convertase subtilisin/kexin type 9 (PCSK9). The agent may be an anti-PCSK9 antibody, such as alirocumab, lodelcizumab, ralpancizumab, or evolocumab, or an antigen-binding portion thereof. Particles targeting PCSK9 may be particularly useful for treating or preventing hypercholesterolemia, atherosclerosis, ischemia, and myocardial infarction, in addition to other conditions and diseases.

The biomolecule may be adrenomedullin, brain-derived neurtrophic factor, erythropoietin, fibroblast growth factor, hepatoma-derived growth factor, glucose-6-phosphate isomerase, keratinocyte growth factor, macrophage migration inhibitory factor, neurotrophin (nerve growth factor, brain-derived neurotrophic factor, neurotrophin-3, neurotrophin-4), platelet-derived growth factor, stem cell factor, thrombopoietin, T-cell growth factor, vascular endothelial growth factor (VEGF-A, VEGF-B, VEGF-C, VEGF-D, placental growth factor (PGF)), or renalase. The agent may comprise an antibody, or antigen-binding portion thereof, that selectively binds adrenomedullin, brain-derived neurtrophic factor, erythropoietin, fibroblast growth factor, hepatoma-derived growth factor, glucose-6-phosphate isomerase, keratinocyte growth factor, macrophage migration inhibitory factor, neurotrophin (nerve growth factor, brain-derived neurotrophic factor, neurotrophin-3, neurotrophin-4), platelet-derived growth factor, stem cell factor, thrombopoietin, T-cell growth factor, vascular endothelial growth factor (VEGF-A, VEGF-B, VEGF-C, VEGF-D, placental growth factor (PGF)), or renalase.

The biomolecule may be soluble tropomyosin receptor kinase B (soluble TrkB). The agent may be an anti-TrkB antibody, or an antigen-binding portion thereof. The biomolecule may be soluble tropomyosin receptor kinase A (soluble TrkA). The agent may be an anti-TrkA antibody, or an antigen-binding portion thereof. The agent may be brain-derived neurotrophic factor.

The biomolecule may be angiopoietin (e.g., angiopoietin 1, angiopoietin 2, angiopoietin 3, or angiopoietin 4) or an angiopoietin like protein (e.g., angiopoietin-like 1, angiopoietin-like 2, angiopoietin-like 3, angiopoietin-like 4, angiopoietin-like 5, angiopoietin-like 6, or angiopoietin-like 7). The agent may be an antibody that selectively binds to angiopoietin (e.g., angiopoietin 1, angiopoietin 2, angiopoietin 3, or angiopoietin 4) or an angiopoietin like protein (e.g., angiopoietin-like 1, angiopoietin-like 2, angiopoietin-like 3, angiopoietin-like 4, angiopoietin-like 5, angiopoietin-like 6, or angiopoietin-like 7).

The biomolecule may be a hedgehog protein (e.g., sonic hedgehog). The agent may be an antibody that selectively binds a hedgehog protein. Particles targeting hedgehog proteins may be particularly useful for treating or preventing cancer, such as pancreatic cancer, cerebellar cancer, and medulloblastomas, in addition to other conditions and diseases.

The biomolecule may be a soluble human leukocyte antigen (HLA) protein (e.g., soluble HLA-A, HLA-B, HLA-C, HLA-D, HLA-E, HLA-F, OR HLA-G (see, e.g., Bassani-Sternberg, M. et al., Proceedings National Academy Sciences USA 107(44):18769 (2010))). The agent may be an antibody that selectively binds a soluble human leukocyte antigen (HLA) protein. The agent may be a soluble killer cell immunoglobulin-like receptor. Particles that target a soluble HLA may be particularly useful for treating or preventing cancer, in addition to other diseases and conditions.

The biomolecule may be a soluble UL16-binding protein isoform (e.g., a soluble RAET1 (ULBP1; RAET1E2), soluble RAET1H (ULBP2), soluble RAET1N (ULBP3), soluble RAET1E (ULBP4), soluble RAET1G (ULBP5), or soluble RAET1L (ULBP6)). The agent may be an antibody that specifically binds a soluble UL16-binding protein isoform, or an antigen-binding portion thereof. The agent may be soluble NKG2D receptor (see, e.g., PCT Patent Application Publication No. WO 2006/024367, hereby incorporated by reference in its entirety).

The biomolecule may be soluble MIC-A or soluble MIC-B (see, e.g., Groh, V. et al., Nature 419(6908):734 (2002)). The agent may be an anti-MIC-A antibody or an anti-MIC-B antibody, or an antigen binding portion of either antibody. The agent may be soluble NKG2D receptor (see, e.g., PCT Patent Application Publication No. WO 2006/024367, hereby incorporated by reference in its entirety).

The agent may be a soluble natural cytotoxicity receptor (see, e.g., Jarahian, M. et al. PloS Pathogens 7(8): e1002195 (2011)).

The biomolecule may be soluble C-type lectin domain family 2 member D (soluble CLEC2D; soluble Lectin Like Transcript-1 (LLT1)) (see, e.g., Chalan, P. et al., PloS One 10(7): e0132436 (2015)). The agent may be an antibody that selectively binds soluble LLT1. Particles that target a soluble LLT1 may be particularly useful for treating or preventing autoimmune diseases, such as rheumatoid arthritis, in addition to other diseases and conditions.

The biomolecule may be soluble CD16 (see, e.g., Hoover, R. G., J Clinical Investigation 95:241 (1995)). The agent may be an antibody that selectively binds a soluble CD16. Particles that target soluble CD16 may be particularly useful for treating or preventing cancer, in addition to other diseases and conditions.

The biomolecule may be plasminogen activator inhibitor-1 (PAI-1), plasminogen activator inhibitor-1 (PAI-2), tissue plasminogen activator, urokinase, plasminogen, thrombin, or α2-macroglobulin. The agent may be an antibody that selectively binds plasminogen activator inhibitor-1 (PAI-1), plasminogen activator inhibitor-1 (PAI-2), tissue plasminogen activator, urokinase, plasminogen, thrombin, or α2-macroglobulin.

The biomolecule may be Factor XII, Factor XIIa, Factor XI, Factor XIa, Factor IX, Factor IXa, Factor X, Factor Xa, Factor VII, Factor VIIa, Factor XIII, Factor XIIIa, Factor V, prothrombin, thrombin, von Willebrand factor, thromboxane A2, fibrinogen, or fibrin. The agent may be an antibody that selectively binds to Factor XII, Factor XIIa, Factor XI, Factor XIa, Factor IX, Factor IXa, Factor X, Factor Xa, Factor VII, Factor VIIa, Factor XIII, Factor XIIIa, Factor V, prothrombin, thrombin, von Willebrand factor, thromboxane A2, fibrinogen, or fibrin.

The biomolecule may be a serpin (e.g., al-antitrypsin, antitrypsin-related protein, α1-antichymotrypsin, kallistatin, protein C inhibitor, transcortin, thyroxine-binding globulin, angiotensinogen, centerin (GCET1), protein Z-related protease inhibitor, vaspin, antithrombin, heparin cofactor II, plasminogen activator inhibitor 1, glia derived nexin (protease nexin I), pigment epithelium derived factor, α2-antiplasmin, complement 1-inhibitor, neuroserpin, plasminogen activator inhibitor, 2SERPINA1, or SERPINA2). The agent may comprise an antibody that selectively binds a serpin, or an antigen-binding portion thereof.

The biomolecule may be soluble ST2. The agent may be interleukin 33 or an antibody that specifically binds soluble ST2 (or a fragment thereof). Particles that target soluble ST2 may be particularly useful for treating or preventing heart disease, myocardial infarction, acute coronary syndrome, and heart failure, in addition to other disease and conditions.

The biomolecule may be myostatin (growth differentiation factor 8 (GDF-8)). The agent may be an anti-myostatin antibody, such as stamulumab or trevogrumab. The agent may be an activin receptor or a myostatin-binding portion thereof, e.g., the agent may be soluble activin type IIB receptor. Particles targeting myostatin may be particularly useful for treating muscular dystrophy, cachexia, sarcopenia, and various forms of muscle loss (such as zero-gravity muscle loss), in addition to other diseases and conditions.

The biomolecule may be ghrelin. The agent may be an anti-ghrelin antibody. Particles targeting ghrelin may be particularly useful for treating or preventing obesity, Prader-Willi syndrome, addiction, alcoholism, and leptin resistance (e.g., genetic leptin resistance).

The biomolecule may be sLR11 (soluble SORL1; soluble SORLA; soluble SORLA1). The agent may be an anti-sLR11 antibody. Particles targeting sLR11 may be particularly useful for treating or preventing obesity, in addition to other diseases and conditions.

The biomolecule may be TGF-β (transforming growth factor beta, e.g., TGF-β1, TGF-β2, or TGF-β3). The agent may be an anti-TGF-β antibody, such as fresolimumab, lerdelimumab, or metelimumab. The agent may comprise the TGF-β binding domain of a TGF-β receptor. The agent may be LTBP₁ (latent-transforming growth factor beta-binding protein 1), 14-3-3-protein epsilon (tyrosine 3-monooxygenase/tryptophan 5-monooxygenase activation protein, epsilon; YWHAE), or eukaryotic translation initiation factor 3 subunit I (EIF3I), each of which binds to TGF-β. Particles targeting TGF-β may be particularly useful for treating or preventing scleroderma, idiopathic pulmonary fibrosis, renal disease, focal segmental glomerulosclerosis, keratoconus, Marfan syndrome, Alzheimer's disease, cognitive decline, traumatic brain injury, muscle wasting, and cancer (e.g., kidney cancer and melanoma), in addition to other diseases and conditions.

The biomolecule may be Wnt (e.g., Wnt1, Wnt2, Wnt2B, Wnt3, Wnt3A, Wnt4, Wnt5A, Wnt5B, Wnt6, Wnt7A, Wnt7B, Wnt8A, Wnt8B, Wnt9A, Wnt9B, Wnt10A, Wnt10B, Wnt11, or Wnt16). The agent may be an anti-Wnt antibody. Particles targeting Wnt may be particularly useful for treating or preventing obesity, type II diabetes, atherosclerosis, calcific aortic valve stenosis, heart attack, heart failure, stroke, and cancer (e.g., breast cancer, colorectal cancer, esophageal cancer, melanoma, prostate cancer, lung cancer, non-small cell lung cancer, mesothelioma, sarcoma, glioblastoma, or ovarian cancer), in addition to other diseases and conditions.

The biomolecule may be a soluble Notch ligand (e.g., soluble Jagged1, soluble Jagged2, soluble Delta-like ligand 1 (DLL1), soluble Delta-like ligand 3 (DLL3), and Delta-like ligand 4 (DLL4)). The agent may be an anti-Notch ligand antibody, such as demcizumab or enoticumab, or a soluble Notch receptor (e.g., soluble NOTCH1, NOTCH2, NOTCH3, or NOTCH4) or a variant thereof. Particles targeting soluble Notch ligands may be particularly useful for treating or preventing atherosclerosis, calcific aortic valve stenosis, heart attack, heart failure, stroke, and cancer (e.g., breast cancer, pancreatic cancer renal cell carcinoma, non-small cell lung cancer, and solid tumors), in addition to other diseases and conditions.

The biomolecule may be a soluble Notch receptor (e.g., soluble NOTCH1, NOTCH2, NOTCH3, or NOTCH4). The agent may be an anti-Notch receptor antibody, such as tarextumab or brontictuzumab, or a soluble Notch ligand. Particles targeting soluble Notch receptors may be particularly useful for treating or preventing atherosclerosis, calcific aortic valve stenosis, heart attack, heart failure, stroke, and cancer (e.g., breast cancer, pancreatic cancer renal cell carcinoma, non-small cell lung cancer, and solid tumors), in addition to other diseases and conditions.

The target may be hydroxyapatite or calcium (e.g., crystalline calcium). The agent may be a chelating agent such as ethylene diamine tetraacetic acid (EDTA), diethylene triamine pentaacetic acid (DTPA), sodium thiosulfate (STS), inositol hexaphosphate, or citric acid. Particles targeting hydroxyapatite or calcium may be particularly useful for treating or preventing atherosclerosis, calcific aortic valve stenosis, and calcific tendinitis, in addition to other diseases and conditions.

In some embodiments, the biomolecule is an autoantibody. An autoantibody is an antibody produced by a subject that specifically binds an antigen produced by the subject. Autoantibodies are associated with many different disease states, including lupus. Additionally, the induction of new autoantibodies may be associated with a therapeutic intervention, e.g., resulting in drug-induced lupus. Thus, a composition comprising a plurality of particles comprising an agent that selectively binds one or more autoantibodies may be used, for example, in a method of treating or preventing lupus (e.g., drug-induced lupus). The biomolecule may be, for example, a double-stranded DNA autoantibody or an anti-nuclear autoantibody.

A particle that targets an autoantibody may comprise an agent that is the antigen of the autoantibody.

The biomolecule may be an anti-β adrenoceptor autoantibody or an anti-M2 muscarinic receptor autoantibody, e.g., for preventing or treating idiopathic dilated cardiomyopathy. In particular, a particle that targets an anti-β adrenoceptor autoantibody or an anti-M2 muscarinic receptor autoantibody may be administered to a subject with Chagas' disease, which correlates with the induction of such autoantibodies (see, e.g., Herda, L. R. et al., Br J Pharmacol 166(3)847 (2012)). The biomolecule may be an anti-alpha-1-adrenergic receptor autoantibody, e.g., for treating or preventing hypertension (see, e.g., Luther, H. P. et al., Hypertension 29(2):678 (1997)). The biomolecule may be an anti-muscarinic type 3 receptor autoantibody, e.g., for use in treating or preventing Sjögren's syndrome (see, e.g., Lee, B. H. et al., PloS One 8(1):e53113 (2013)).

Autoantibodies against hormones and cytokines may buffer the concentration of hormones and cytokines, for example by reversibly binding to them to control the concentration of free, active species. Deviations from healthy autoantibody levels may contribute to diseases arising from loss of cytokine or hormonal homeostasis. For example, anti-IFNγ autoantibodies may induce disseminated non-tuberculosis mycobacterial infections, anti-IL-17 autoantibodies are associated with the development of chronic mucosal candidiasis, and anti-IL-6 autoantibodies are associated with severe staphylococcal or streptococcal infections. Autoantibodies to the hunger hormone ghrelin may mediate the effective concentration of ghrelin available to bind to ghrelin receptor GHSR1.

In some embodiments, the biomolecule is an autoantibody. For example, the autoantibody may be an anti-IFNγ, anti-IL-17, anti-IL-6, or anti-ghrelin autoantibody. In some embodiments, the agent is the natural ligand of an autoantibody (e.g., an antigen targeted by the autoantibody). For example, the agent may be IFNγ, IL-17, IL-6, or ghrelin. In some embodiments, the invention relates to a method of treating a patient with a disease of dysregulation of a cytokine, such as an autoimmune disease. In some embodiments, the invention relates to a method of treating a patient with metabolic disorder, such as obesity.

Activin binding to activin type IIB receptor ActRIIB leads to muscle wasting in models of cachexia. Excessive activin levels in serum are associated with muscle wasting and fibrosis in models of cachexia, which may be reversed by antibodies that block activin A and B/ActRIIB signalling, and elevated activin levels are found in serum of cancer patients. Sarcopenia is a progressive condition of loss of muscle mass in aging and has also been associated with excessive activin signalling. The biomolecule may thus be activin (e.g., activin A or activin B). The agent may be a natural ligand for an activin, such as an activin receptor protein such as ActRIIB or a variant thereof, or an antibody against an activin. The agent may be myostatin. In some embodiments, the invention relates to a method of treating a patient a muscle-wasting disease, such as cachexia or sarcopenia.

A skilled artisan will also appreciate that the particles described herein are also useful for scavenging a wider variety of targets whose biological activity may be, e.g., undesirable. For example, the particles can be engineered to bind to components of viral capsids or envelopes to thereby sequester virus from the blood of a subject. The particles may be, in some embodiments, engineered to bind and sequester toxins (e.g., bacterial toxins, plant toxins, and zootoxins, such as one or more components of snake venom) in the circulation of a subject. In some embodiments, the particles can be engineered to bind to and sequester small molecules (e.g., psychoactive drugs or small molecular toxins) from the circulation of a subject. In such embodiments, the particles can be useful to remove toxins from the body, e.g., following a snake or insect bite. In some embodiments, the particles can be used for treating, preventing, delaying the onset, or reducing the severity of, anaphylactic shock in a subject (e.g., by scavenging the antigen giving rise to the anaphylactic immune response).

In some embodiments, the target is associated with a virus, e.g., a viral structural protein (such as a viral capsid or viral envelope protein) that is bound by the agent. In such embodiments, the particles are useful as anti-viral therapies, e.g., for a subject infected with a virus or at risk of being infected with a virus. A virus may be an enveloped or non-enveloped virus.

In some embodiments, the soluble biomolecule is a small molecule or macromolecule. In some embodiments, the longest dimension of the soluble biomolecule is no greater than 600 nm (e.g., less than 550, 500, 450, 400, 350, 300, 250, 200, 150, 100, 50, or 25 nm). For example, the biomolecule may have a molecular radius of about 1 Å to about 1 μm, such as about 1 Å to about 100 nm, about 1 Å to about 20 nm, about 1 nm to about 1 μm, about 1 nm to about 100 nm, or about 1 nm to about 20 nm. The biomolecule may have a molecular weight of about 3 amu to about 10⁷ amu, such as about 100 amu to about 10⁷ amu, about 3 amu to about 10⁶ amu, about 3 amu to about 10⁵ amu, about 100 amu to about 10⁶ amu, or about 400 amu to about 10⁶ amu. The biomolecule may have a molecular weight of about 10⁵ amu to about 10⁷ amu.

The terms “specific binding,” “specifically binds,” “selective binding,” “selectively binds,” and like grammatical terms, as used herein, refer to two molecules forming a complex that is relatively stable under physiologic conditions. Typically, binding is considered specific when the association constant (kₐ) is higher than 10⁶M⁻¹s⁻¹. Thus, a first member of a specific binding pair can specifically bind to the second member of the binding pair with a kₐ of at least (or greater than) 10⁶M⁻¹s⁻¹ (e.g., at least or greater than 10⁷, 10⁸, 10⁹, 10¹⁰, 10¹¹, 10¹², 10¹³, 10¹⁴, or 10¹⁵ M⁻¹s⁻¹ or higher). In some embodiments, a selective interaction has a dissociation constant (k_{d}) of less than or equal to 10⁻³ s⁻¹ (e.g., 8×10⁻⁴, 5×10⁻⁴, 2×10⁻⁴, 10⁻⁴, or 10⁻⁵ s⁻¹).

Specific binding does not refer to an interaction that is primarily driven by a non-specific electrostatic interaction or a non-specific hydrophobic interaction, which may have a favorable association constant. For example, nucleic acids, which are negatively charged, may bind to a cationic particle with a favorable association constant, independent of a specific interaction, and such binding is not “specific binding” as defined herein. Similarly, a lipid may bind to a hydrophobic particle with a favorable association constant, independent of a specific interaction, and such binding is not “specific binding” as defined herein.

In some embodiments, the biomolecule and the particle have the same charge at physiological pH (˜7.4). For example, the biomolecule may have a negative charge and the particle may have a negative charge or the biomolecule may have a positive charge and the particle may have a positive charge. In some embodiments, the biomolecule and the particle have opposite charges at physiological pH. For example, the biomolecule may have a positive charge and the particle may have a negative charge or the biomolecule may have a negative charge and the particle may have a positive charge. In some embodiments, the biomolecule has a neutral charge at physiological pH and/or the particle has a neutral charge at physiological pH.

The biomolecule may have an isoelectric point of about 0 to about 14. Nucleic acids have an isoelectric point of about 4 to about 7, and thus, the biomolecule may have an isoelectric point of about 4 to about 7. Proteins generally have an isoelectric point of about 4 to about 10, and thus, the biomolecule may have an isoelectric point of about 4 to about 10. Nevertheless, unmodified peptides and proteins may have isoelectric points ranging from about 2.5 (based on aspartate; pI˜2.8) to about 11 (based on arginine; pI˜11), although proteins with isoelectric points falling outside of this range are known. Accordingly, the biomolecule may have an isoelectric point ranging from about 2.5 to about 11. Secreted proteins and the soluble, extracellular portions of membrane proteins typically have a slight negative charge at physiological pH, and thus, the biomolecule may have an isoelectric point of about 4 to about 7, such as about 4 to about 6. The biomolecule may have an isoelectric point of about 0 to about 4, about 2 to about 6, about 4 to about 8, about 6 to about 10, about 8 to about 12, or about 10 to about 14. The biomolecule may have an isoelectric point of about 0 to about 2, about 1 to about 3, about 2 to about 4, about 3 to about 5, about 4 to about 6, about 4 to about 6, about 5 to about 7, about 6 to about 8, about 7 to about 9, about 8 to about 10, about 9 to about 11, about 10 to about 12, about 11 to about 13, or about 12 to about 14.

In some embodiments, a selective interaction has a K_{D} of less than 10⁻⁸, 10⁻⁹, 10⁻¹⁰, 10⁻¹¹, or 10¹² M. The equilibrium constant K_{D} is the ratio of the kinetic rate constants—k_{d}/kₐ. In some embodiments, a selective interaction has a K_{D} of less than 1×10⁻⁹M.

As used herein, the term “interaction,” when referring to an interaction between two molecules, refers to the physical contact (e.g., binding) of the molecules with one another. Generally, such an interaction results in an activity (which produces a biological effect) of one or both of said molecules. To inhibit such an interaction results in the disruption of the activity of one or more molecules involved in the interaction.

As used herein, the term “inhibiting” and grammatical equivalents thereof refer to a decrease, limiting, and/or blocking of a particular action, function, or interaction. In one embodiment, the term refers to reducing the level of a given output or parameter to a quantity (e.g., the background level of the interaction between two members of a specific binding pair) which is at least 10%, 15%, 20%, 25%, 30%, 35%, 40%, 45%, 50%, 55%, 60%, 65%, 70%, 75%, 80%, 85%, 90%, 95%, 99% or less than the quantity in a corresponding control. A reduced level of a given output or parameter need not, although it may, mean an absolute absence of the output or parameter. The invention does not require, and is not limited to, methods that wholly eliminate the output or parameter. Substantial inhibition can be, e.g., at least 50% (e.g., 55, 60, 65, 70, 75, 80, 85, 90, or 95% or greater) inhibition of an interaction between two biomolecules (e.g., the first and second members of a binding pair).

Methods for detecting an interaction or measuring the affinity of one biomolecule for another are known in the art. For example, the binding of two biomolecules can be detected and/or quantified using a variety of techniques such as, but not limited to, BioLayer Interferometry (BLI), Western blot, dot blot, surface plasmon resonance method (SPR), enzyme-linked immunosorbent assay (ELISA), AlphaScreen® or AlphaLISA® assays, or mass spectrometry based methods.

In some embodiments, binding can be assayed using any SPR-based assays known in the art for characterizing the kinetic parameters of the interaction of two biomolecules. Any SPR instrument commercially available including, but not limited to, BIAcore Instruments (Biacore AB; Uppsala, Sweden); 1Asys instruments (Affinity Sensors; Franklin, Mass.); IBIS system (Windsor Scientific Limited; Berks, UK), SPR-CELLIA systems (Nippon Laser and Electronics Lab; Hokkaido, Japan), and SPR Detector Spreeta (Texas Instruments; Dallas, Tex.) can be used in the methods described herein. (See, e.g., Mullett et al., Methods 22:77-91(2000); Dong et al., Reviews in Mol Biotech 82:303-323(2002); Fivash et al., Curr Opin Biotechnol 9:97-101(1998); and Rich et al., Curr Opin Biotechnol 11:54-61(2000)).

In some embodiments, biomolecular interactions between two biomolecules can be assayed using BLI on an Octet (ForteBio Inc.). BLI is a label-free optical analytical technique that senses binding between a ligand that is immobilized on a biosensor tip and an analyte in solution by measuring the change in the thickness of the protein layer on the biosensor tip in real-time.

In some embodiments, AlphaScreen (PerkinElmer) assays can be used to characterize binding of two biomolecules. The acronym ALPHA stands for Amplified Luminescent Proximity Homogeneous Assay. AlphaScreen is a bead-based proximity assay that senses binding between molecules attached to donor and acceptor beads by measuring the signal produced by energy transfer between the donor and acceptor beads. (See, e.g., Eglen et al., Curr Chem Genomics 1:2-10(2008)).

In some embodiments, AlphaLISA® (PerkinElmer) assays can be used to characterize binding of two biomolecules. AlphaLISA is modified from the AlphaScreen assay described above to include europium-containing acceptor beads and functions as an alternative to traditional ELISA assays. (See, e.g., Eglen et al., Curr Chem Genomics 1:2-10(2008)).

A variety of immunoassay techniques, including competitive and non-competitive immunoassays, can be used. The term “immunoassay” encompasses techniques including, without limitation, flow cytometry, FACS, enzyme immunoassays (EIA), such as enzyme multiplied immunoassay technique (EMIT), enzyme-linked immunosorbent assay (ELISA), IgM antibody capture ELISA (MAC ELISA), and microparticle enzyme immunoassay (META), furthermore capillary electrophoresis immunoassays (CEIA), radio-immunoassays (MA), immunoradiometric assays (IRMA), fluorescence polarization immunoassays (FPIA), and chemiluminescence assays (CL). If desired, such immunoassays can be automated. Immunoassays can also be used in conjunction with laser induced fluorescence. Liposome immunoassays, such as flow-injection liposome immunoassays and liposome immunosensors, are also suitable for use in the present invention. In addition, nephelometry assays, in which, for example, the formation of biomolecular complexes results in increased light scatter that is converted to a peak rate signal as a function of the marker concentration, are suitable for use in the methods of the present invention. In a preferred embodiment of the present invention, the incubation products are detected by ELISA, RIA, fluoro immunoassay (FIA) or soluble particle immune assay (SPIA).

In some embodiments, binding of two biomolecules can be assayed using thermodenaturation methods involving differential scanning fluorimetry (DSF) and differential static light scattering (DSLS).

In some embodiments, binding of two biomolecules can be assayed using a mass spectrometry based method such as, but not limited to, an affinity selection coupled to mass spectrometry (AS-MS) platform. This is a label-free method where the protein and test compound are incubated, unbound molecules are washed away and protein-ligand complexes are analyzed by MS for ligand identification following a decomplexation step.

In some embodiments, binding of two biomolecules can be quantitated using, for example, detectably labeled proteins such as radiolabeled (e.g., ³²P, ³⁵S, ¹⁴C or ³H), fluorescently labeled (e.g., FITC), or enzymatically labeled biomolecule, by immunoassay, or by chromatographic detection.

In some embodiments, the present invention contemplates the use of fluorescence polarization assays and fluorescence resonance energy transfer (FRET) assays in measuring, either directly or indirectly, the degree of interaction between two biomolecules.

### II. PARTICLES

As used herein, the term “particle” refers to a small mass that can comprise any material, such as alumina, metal (e.g., gold or platinum), glass, silica, latex, plastic, agarose, polyacrylamide, methacrylate or any polymeric material, and be of any size and shape. In some embodiments, the particle or particles comprise silicon. (See, e.g., International Patent Application Publication Nos. WO 2013/011764, WO 2013/029278, and WO 2014/151381, and U.S. Patent Application Publication No. 2014/0271886, the disclosures of each of which are incorporated by reference in their entirety). In some embodiments, the particles comprise or consist of starch (see, e.g., International Patent Application Publication No. WO 2010/084088). In some embodiments, the particle or particles are composed of nucleic acid (e.g., naturally-occurring or non-naturally occurring nucleic acid). Methods for making such nucleic acid-based microscopic structures are known in the art and are described in, e.g., Douglas et al., Nucl Acids Res 37(15):5001-5006 (2009); Douglas et al., Nature 459(7245):414-428 (2009); Voigt et al., Nat Nanotechnol 5(3):200-203 (2010); and Endo et al., Curr Protoc Nucleic Acid Chem Chapter 12(Unit 12.8) (2011).

In preferred embodiments, the particle is insoluble in aqueous solution (e.g., the particle may be insoluble in water, blood serum, blood plasma, extracellular fluid, and/or interstitial fluid). For example, a particle may be separated from aqueous solution by centrifuging a solution comprising the particle, e.g., at speeds that are sufficient to separate the cells of a cell suspension from the aqueous solution of the cell suspension. Nevertheless, a particle may readily exist as a suspension in aqueous solution, e.g., mild shaking or vortexing of a plurality of particles in aqueous solution is sufficient to suspend the particles in the solution. In some embodiments, the particle is not a hydrogel. In some embodiments, the particle does not comprise a hydrogel. In some embodiments, the particle does not comprise a polymer.

A particle is preferably large enough to bind to more than one biomolecule and inhibit the interaction of more than one bound biomolecule with a binding partner. For example, a particle may be about 50 nm to about 10 Å particle may be 1 μm to 5 μm in size, 1.2 μm to 4 μm, 1.5 μm to 4 or 2 μm to 4 μm.

Particles with sizes less than 300 nm, such as less than 200 nm or less than 150 nm, are preferred for applications in which the particles are intended to enter and/or exit the vasculature of a subject, such as particles that may be administered by subcutaneous injection. Nevertheless, larger particles are similarly well-suited for subcutaneous injection for methods in which the particles are not intended to enter the vasculature. Particles with sizes of about 1 μm to about 5 μm are preferable for applications in which the particles are intended to circulate within the vasculature of a subject, e.g., following intravenous administration. Particles with sizes greater than 5 μm may be preferable for applications in which the particles are intended to reside at the site in which they are implanted, such as within or adjacent to a tumor; however, particles smaller than 5 μm may also be suitable for implantation. Particles of any size may be utilized for in vitro applications.

Also featured herein are collections of particles. In some embodiments, the plurality of particles has a narrow or broad polydispersity. As used herein, “polydispersity” refers to the range of sizes of particles within a particular particle population. That is, an extremely polydisperse population might involve particles having a mean size of, say, 1 μm with individual particles ranging from 0.1 to 4 In some embodiments, a “narrow polydispersity” is preferred. That is, given a particular mean particle size, it is presently preferred that individual particles in the population differ by no more than ±20%, preferably no more than ±15%, and most preferably at present no more than ±10% from the mean particle size. More specifically, a particle population preferably has a mean particle size of about 0.5 to about 2 μm, more preferably at present from about 0.8 to about 1.5 μm. Thus, if a mean particle size of 1 μm is selected, individual particles in the population would most preferably be within the range of from about 0.8 to about 1.2 μm. In some embodiments, the particle population has a mean particle size of about 0.3 to about 1 μm, e.g., about 0.4 to about 0.9, about 0.5 to about 0.9, about 0.4 to about 0.8, about 0.5 to about 0.7, about 0.3 to about 0.9, or about 0.3 to about 0.7 μm. In some embodiments, the particle population has a mean particle size of about 1 μm to about 10 μm, e.g., about 1.1 μm to about 4.8, about 1.2 μm to about 4.6, about 1.4 μm to about 4.4, about 1.6 μm to about 4.2, about 1.8 μm to about 4.0, or about 2.0 μm to about 3.8 μm.

In some embodiments, the disclosure features a collection or plurality of particles having a defined mean particle size. As used herein, “mean particle size” is arrived at by measuring the size of individual particles and then dividing by the total number of particles. The determination of mean particle size is well known in the art. Typically, the longest average dimension of the particles is no greater than 4 μm. In some embodiments, the longest average dimension of the particles is no greater than 3.9 (e.g., no greater than 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, or 1)μm. In some embodiments, the longest average dimension of the particles is no greater than 2.5 μm, 2 μm, 1.5 μm, or 1.25 μm. In some embodiments, the longest average dimension of the particles is at least 1 μm, but no greater than 4 μm. In some embodiments, the longest average dimension of the particles is at least 1 μm, but no greater than 2 μm. In some embodiments, the longest average dimension of the particles is at least 1 μm, but no greater than 1.5 μm. In some embodiments, the longest average dimension of the particles is at least 0.5 μm (e.g., at least 0.6, 0.7, 0.8, 0.9, 1, 1.1, 1.2, 1.3, 1.4, or 1.5 μm), but no greater than 4 μm (e.g., no greater than 3.9 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2, 1.9, 1.8, 1.7, or 1.6 μm).

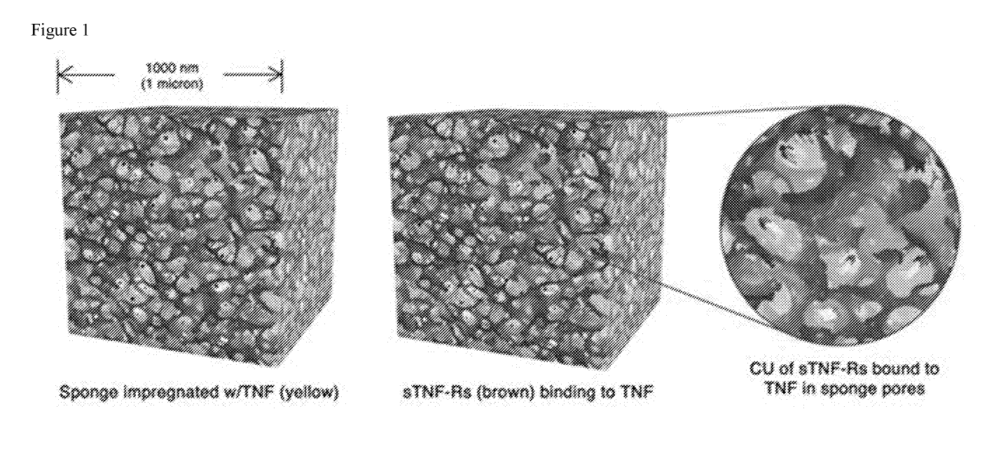

In some embodiments, the particles are nanoparticles. In some embodiments, the longest average dimension of the particles is no greater than 900 nm (e.g., 850, 800, 750, 700, 650, 600, 550, 500, 450, 400, 450, 400, 350, 300, 250, 200, or 150 nm). In some embodiments, a particle is shaped and sized to circulate in the blood or vasculature (e.g., arteries, veins, and capillaries) of a subject (e.g., a human subject). Exemplary particle designs are set forth in FIGS. 1 to 6.

In some embodiments, the longest dimension of the particle is about 50 nm to about 5 μm, such as about 100 nm to about 4.5 μm, about 200 nm to about 4 μm, about 300 nm to about 3.5 μm, about 300 nm to about μm, or about 400 nm to about 3 μm. In some embodiments, the shortest dimension of the particle is at least about 300 nm, such as about 300 nm to about 4 μm or about 400 nm to about 3 μm.

In some embodiments, a plurality of the particles are polyhedral, e.g., cubic. In some embodiments, a plurality of the particles are spherical. In some embodiments, any of the particles described herein can be porous. Such porous particles comprise an outer surface and inner surfaces of the pores of the particle. The agent can be, e.g., immobilized on the inner surfaces. In some embodiments, a plurality of pores have a cross-sectional dimension of at least 50 nm. In some embodiments, a plurality of pores have a cross-sectional dimension of at least 100 nm. Porous nanoparticles have been described in, e.g., U.S. Patent Application Publication Nos. 20140199352, 20080277346, and 20040105821, the disclosures of each of which are incorporated by reference in their entirety. Spherical particles are described in, e.g., U.S. Pat. Nos. 8,778,830 and 8,586,096, each of which is hereby incorporated by reference.

In some embodiments, spherical particles can further comprise two intersecting ridges extending from the spherical surface of the particle, wherein the longest dimension of each of the structures is no greater than 4 μm (e.g., no greater than 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, or 1 μm), and wherein the ridges are sized and oriented: (i) to inhibit the agent immobilized on the surface of the spherical particle from binding to, or activating, a cell surface receptor protein and/or (ii) when the soluble biomolecule is bound to the agent, to inhibit the interaction of the soluble biomolecule and a second member of a specific binding pair of which the soluble biomolecule is the first member.

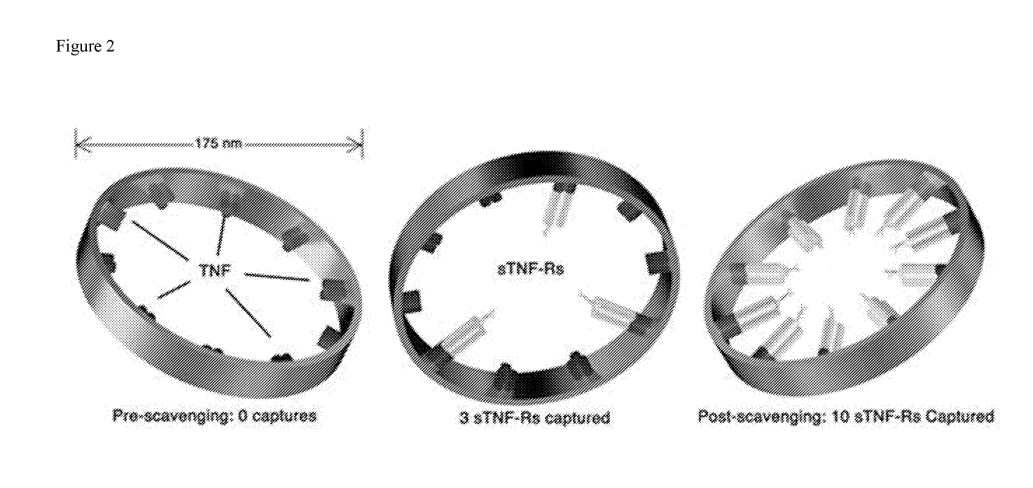

In some embodiments, a plurality of particles are toroidal. In such embodiments, the agent can be immobilized on an inner circumferential surface of the particle (e.g., around the hole—see FIG. 2). In some embodiments, the diameter of the particle is no greater than 4 μm (e.g., 3.9, 3.8, 3.7, 3.6, 3.5, 3.4, 3.3, 3.2, 3.1, 3.0, 2.9, 2.8, 2.7, 2.6, 2.5, 2.4, 2.3, 2.2, 2.1, 2, 1.9, 1.8, 1.7, 1.6, 1.5, 1.4, 1.3, 1.2, 1.1, or 1 μm). In some embodiments, the diameter of the particle is no greater than 900 nm (e.g., 850, 800, 750, 700, 650, 600, 550, 500, 450, 400, 350, 300, 200, or 150 nm).

In some embodiments, the particles described herein are dendritic. Such particles are described in, e.g., Du et al., Small 11(4):392-413 (2015); Siegwart, D. J. et al., Proceedings National Academy Sciences USA 108(32):12996 (2011); U.S. Pat. Nos. 5,814,272 and 7,932,311; and U.S. Patent Application Publication No. 20040166166, the disclosures of each of which are hereby incorporated by reference herein. As elaborated on below, in some embodiments the geometry of the dendritic particles is such that the agent immobilized on the inner surface of the particle has a reduced, or substantially reduced, ability to interact with a biomolecule on the surface of a cell and/or the soluble biomolecule bound to the particle by virtue of the agent has a reduced, or substantially reduced, ability to interact with its cognate ligand (the second member of the specific binding pair).

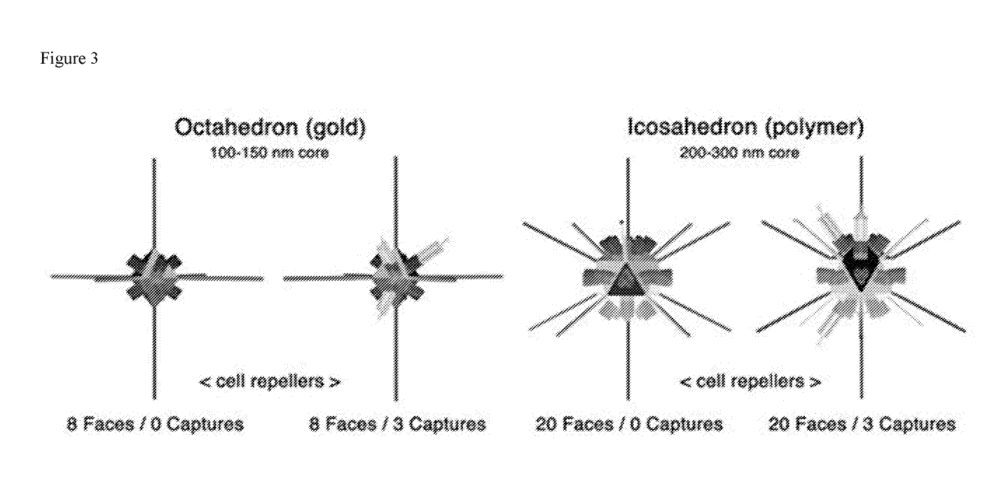

In some embodiments, a plurality of particles are polyhedral, e.g., octahedral or icosahedral (see, e.g., FIG. 3), whether regular or irregular. The particles may comprise at least one protrusion from at least one of their vertices (see, e.g., FIG. 3). The particles may comprise more than one (e.g., 2, 3, 4, 5, 6, 7, or 8 or more) protrusion from their vertices. Such protrusions can be, e.g., sized and/or oriented: (i) to inhibit the agent immobilized on the surface of the spherical particle from binding to, or activating, a cell surface receptor protein and/or (ii) when the soluble biomolecule is bound to the agent, to inhibit the interaction of the soluble biomolecule and a second member of a specific binding pair of which the soluble biomolecule is the first member.

A particle may comprise void space, referred to as a “void” or “voids” herein. A void is the space in a particle that is filled by a fluid (e.g., a liquid, which may comprise a biomolecule, or a gas, such as when a particle is dried) or by empty space (e.g., when a particle is in a vacuum, such as after lyophilization). The void volume of a particle may include, for example, the pore volume of a particle and/or the volume of the interior of a hollow core/shell particle, the lumen of a tube, torus, or ring.

In some embodiments, a particle is configured such that blood plasma may freely enter and/or exit the void space of the particle, e.g., when the particle is located in the vasculature of a subject. In some embodiments, a particle is configured such that blood serum may freely enter and/or exit the void space of the particle, e.g., when the particle is located in the vasculature of a subject. In preferred embodiments, a particle is configured such that blood cells cannot enter the void space of the particle. In some embodiments, a particle is configured such that platelets cannot enter the void space of the particle. Nevertheless, a particle may allow for a platelet to enter its void space, e.g., when the particle is configured for use in vitro or when the particle is configured to bind a virus, bacterium, protist, fungal or yeast cell, or other large target, such as a target sized from about 100 nm to about 2 μm.

In some embodiments, a particle is configured such that extracellular fluid may freely enter and/or exit the void space of the particle. In some embodiments, a particle is configured such that interstitial fluid may freely enter and/or exit the void space of the particle. In some embodiments, a particle is configured such that cerebrospinal fluid may freely enter and/or exit the void space of the particle.

The volume of the void space in a particle is preferentially large enough to accommodate more than one biomolecule, e.g., the total void volume of a particle is preferentially large enough to accommodate each biomolecule that is bound to the particle. Nevertheless, a void may be smaller than the total volume of each bound biomolecule so long as the particle is capable of inhibiting interactions between each bound biomolecule and the second members of the binding pairs that include each biomolecule. For example, a particle may need only sequester a binding site of a biomolecule to inhibit interactions between the biomolecule and a second member of a binding pair, and such a particle may contain a void volume that accommodates the binding site of each biomolecule but that allows for other portions of one or more biomolecules to project outward from the void space.

In some embodiments, a particle may comprise about 5% to about 95% void space. A particle comprising protrusions may comprise little or no void space, e.g., because the protrusions may inhibit interactions between bound biomolecule and a second member of a binding pair. A particle comprising a tube may comprise a large amount of void space, e.g., because a tube may comprise a large internal volume relative to the thickness of the walls of the tube. Nevertheless, the void volume of particles with similar geometries may comprise varying amounts of void volume, e.g., tubes comprising walls of the same thickness may vary substantially in void volume percentage depending on tube diameter.

A particle may comprise 0% to about 40% void space, about 20% to about 60% void space, about 40% to about 80% void space, or about 60% to 100% void space. A particle may comprise 0% to about 20% void space, about 10% to about 30% void space, about 20% to about 40% void space, about 30% to about 50% void space, about 40% to about 60% void space, about 50% to about 70% void space, about 60% to about 80% void space, about 70% to about 90% void space, or about 80% to 100% void space. A particle may comprise 0% to about 10% void space, about 5% to about 15% void space, about 10% to about 20% void space, about 15% to about 25% void space, about 10% to about 20% void space, about 15% to about 25% void space, about 10% to about 20% void space, about 15% to about 25% void space, about 10% to about 20% void space, about 15% to about 25% void space, about 20% to about 30% void space, about 25% to about 35% void space, about 30% to about 40% void space, about 35% to about 45% void space, about 40% to about 50% void space, about 45% to about 55% void space, about 50% to about 60% void space, about 55% to about 65% void space, about 60% to about 70% void space, about 65% to about 75% void space, about 70% to about 80% void space, about 75% to about 85% void space, about 80% to about 90% void space, about 85% to about 95% void space, or about 90% to 100% void space.

The particle may comprise a neutral charge at physiological pH (e.g., ˜7.4). The particle may comprise a slightly negative or slightly positive charge at physiological pH. The surface of a particle (e.g., outer surface) may comprise a slightly negative or slightly positive charge at physiological pH. In preferred embodiments, the surface of a particle (e.g., outer surface) comprises a slightly negative or neutral charge at physiological pH. The isoelectric point of the particle may be about 5 to about 9, preferably about 6 to about 8. Particles comprising a nucleic acid may have an isoelectric point of about 4 to about 7. In some embodiments, the isoelectric point of the particle is less than 7.4, i.e., such that the particle has a net negative charge at physiological pH. For example, the isoelectric point of the particle may be about 6.0 to about 7.4, such as about 6.4 to about 7.4. A particle comprising a net negative charge at physiological pH is less likely to interact with eukaryotic cells (e.g., mammalian cells) because eukaryotic cells generally comprise cell membranes with a net negative charge. A particle preferably does not comprise sufficient charge (and/or charge density) to engage in non-specific interactions with other charged molecules.

### III. Particles Comprising Pores

In some embodiments, the material used to make the particles (e.g., silicon) may have a porosity of about 40% to about 95%, such as about 60% to about 80%. Porosity, as used herein, is a measure of the void spaces in a material, and is a fraction of the volume of voids over the total volume of the material. In certain embodiments, the carrier material has a porosity of at least about 10%, at least about 20%, at least about 30%, at least about 40%, at least about 50%, at least about 60%, at least about 70%, at least about 80%, or even at least about 90%. In particular embodiments, the porosity is greater than about 40%, such as greater than about 50%, greater than about 60%, or even greater than about 70%.

In certain embodiments, the agent is distributed to a pore depth from the surface of the material of at least about 0.005 μm, at least 0.05 μm, at least about 0.1 μm, at least about 0.2 μm, at least about 0.3 μm, at least about 0.4 μm, at least about 0.5 μm, at least about 0.6 μm, or at least about 0.7 μm. In certain embodiments, the agent is distributed in the pores of the carrier material substantially uniformly.

The agent may be loaded into the particle to a depth which is measured as a ratio to the total width of the particle. In certain embodiments, the agent is distributed to a depth of at least about 10% into the particle, to at least about 20% into the particle, at least about 30% into the particle, at least about 40% into the particle, at least about 50% into the particle, or at least about 60% into the particle.

Methods for immobilizing an agent on a porous particle are known, including methods for both immobilizing an agent to a first surface of a particle and immobilizing a different molecule (e.g., coating) to a second surface of the particle (see, e.g., Cauda, V. et al., J. Am. Chem. Soc. 131(32):11361-11370 (2009) and Guan, B. et al., Langmuir, 27(1):328-334 (2011), each of which is hereby incorporated by reference in its entirety). Further, such methods are generally applicable for the manufacture of any of the particles described herein.

The pore size may be preselected to the dimensional characteristics of the agent and target biomolecule to control the release of the biomolecule. Typically, pore sizes that are too small preclude loading of the agent and/or binding of the biomolecule. For example, the average pore diameter for a material may be selected from larger pores, e.g., 15 nm to 40 nm, for high molecular weight molecules, e.g., 200,000-500,000 amu, and smaller pores, e.g., 2 nm to 10 nm, for molecules of a lower molecular weight, e.g., 10,000-50,0000 amu. For instance, average pore sizes of about 6 nm in diameter may be suitable for molecules of molecular weight around 14,000 to 15,000 amu such as about 14,700 amu. Average pore sizes of about 10 nm in diameter may be selected for molecules of molecular weight around 45,000 to 50,000 amu such as about 48,000 amu. Average pore sizes of about 25-30 nm in diameter may be selected for molecules of molecular weight around 150,000 nm.

The pore size may be preselected to be adapted to the molecular radii of the agent or biomolecule. For instance, average pore sizes of about 25 nm to about 40 nm in diameter may be suitable for molecules with a largest molecular radius from about 6 nm to about 8 nm. Molecular radii may be calculated by any suitable method such as by using the physical dimensions of the molecule based on the X-ray crystallography data or using the hydrodynamic radius which represents the solution state size of the molecule. As the solution state calculation is dependent upon the nature of the solution in which the calculation is made, it may be preferable for some measurements to use the physical dimensions of the molecule based on the X-ray crystallography data. As used herein the largest molecular radius reflects half of the largest dimension of the therapeutic agent.

In certain embodiments, the average pore diameter is selected to limit the aggregation of molecules, e.g., proteins, within a pore. It would be advantageous to prevent biomolecules such as proteins from aggregating in a carrier material as this is believed to impede the controlled release of molecules into a biological system. Therefore, a pore that, due to the relationship between its size and the size of a biomolecule, allows, for example, only one biomolecule to enter the pore at any one time, will be preferable to a pore that allows multiple biomolecules to enter the pore together and aggregate within the pore. In certain embodiments, multiple biomolecules may be loaded into a pore, but due to the depth of the pore, the proteins distributed throughout this depth of the pore will aggregate to a lesser extent.

### IV. Particles Comprising at Least One Tube

In some embodiments, the particle comprises at least one tube. In preferred embodiments, the at least one tube comprises one open end or two open ends.

The term “tube” refers to a three-dimensional shape having a length along an axis (e.g., a one-dimensional axis in Cartesian space) and an internal cavity, lumen, void, or reservoir along the length of the shape. In some embodiments, perpendicular cross sections along the axis of the tube have a substantially identical shape and/or size. The term “cross section,” as used in relation to a tube, refers to a two-dimensional cross section that is perpendicular to the axis of the tube. A larger structure may comprise a tube. For example, a syringe comprises a tube, but the tube does not comprise the syringe plunger. A particle or other article may comprise more than one tube. For example, a syringe may comprise two tubes corresponding to the syringe needle and the syringe barrel, or to parallel barrels of a double syringe (e.g., used for epoxy compositions).

A tube may have a diameter, which is the average length of the line segments that are perpendicular to the axis of the tube, wherein each line segment is bounded by two points on the outer surface of the tube. A tube may have a width and height, wherein the width of the tube is the longest line segment defined by two points on the outer surface of the tube that is perpendicular to the axis of the tube, and the height of the tube is the line segment defined by two points on the outer surface of the tube that is perpendicular to both the axis of the tube and the line segment defining the width of the tube.

A tube may have an internal diameter, which is the average length of the line segments that are perpendicular to the axis of the tube, wherein each line segment is bounded by two points on the inner surface of the tube. A tube may have an internal width and internal height, wherein the internal width of the tube is the longest line segment defined by two points on the outer surface of the tube that is perpendicular to the axis of the tube, and the internal height of the tube is the line segment defined by two points on the outer surface of the tube that is perpendicular to both the axis of the tube and the line segment defining the width of the tube.

A tube may be substantially cylindrical. The tube may have a substantially circular cross section. The cross section of the tube may be an ellipsoid, such as a circle.

The cross section of the tube may be a polygon, such as a regular polygon. The cross section of the tube may be a triangle, such as an equilateral triangle. The cross section of the tube may be a quadrilateral, such as a regular quadrilateral, a rectangle, or a square. The cross section of the tube may be a pentagon, such as a regular pentagon. The cross section of the tube may be a hexagon, such as a regular hexagon. A tube may be a triangular tube, square tube, pentagonal tube, hexagonal tube, heptagonal tube, or octahedral tube.

The length of a tube may be about 5 nm to about 5 μm, such as about 5 nm to about 4 μm, about 5 nm to about 3 μm, about 5 nm to about 2 μm, or about 5 nm to about 1 μm. The length of a tube may be about 50 nm to about 5 μm, such as about 50 nm to about 4 μm, about 50 nm to about 3 μm, about 50 nm to about 2 μm, or about 50 nm to about 1 μm. The length of a tube may be about 100 nm to about 5 μm, such as about 100 nm to about 4 μm, about 100 nm to about 3 μm, about 100 nm to about 2 μm, or about 100 nm to about 1 μm. The length of a tube may be about 300 nm to about 5 μm, such as about 300 nm to about 4 μm, about 300 nm to about 3 μm, about 300 nm to about 2 μm, or about 300 nm to about 1 μm. The length of a tube may be about 500 nm to about 5 μm, such as about 500 nm to about 4 μm, about 500 nm to about 3 μm, about 500 nm to about 2 μm, or about 500 nm to about 1 μm.

The diameter, width, and/or height of a tube may be about 5 nm to about 5 μm, such as about 5 nm to about 4 μm, about 5 nm to about 3 μm, about 5 nm to about 2 μm, about 5 nm to about 1 μm, about 5 nm to about 900 nm, about 5 nm to about 800 nm, about 5 nm to about 700 nm, about 5 nm to about 600 nm, about 5 nm to about 500 nm, about 5 nm to about 400 nm, about 5 nm to about 300 nm, about 5 nm to about 200 nm, or about 5 nm to about 100 nm. The diameter, width, and/or height of a tube may be about 50 nm to about 5 μm, such as about 50 nm to about 4 μm, about 50 nm to about 3 μm, about 50 nm to about 2 μm, about 50 nm to about 1 μm, about 50 nm to about 900 nm, about 50 nm to about 800 nm, about 50 nm to about 700 nm, about 50 nm to about 600 nm, about 50 nm to about 500 nm, about 50 nm to about 400 nm, about 50 nm to about 300 nm, about 50 nm to about 200 nm, or about 50 nm to about 100 nm.

The internal diameter, internal width, and/or internal height of a tube are preferentially large enough to accommodate both the agent and the biomolecule. The internal diameter, internal width, and/or internal height of a tube are preferentially small enough to inhibit a cell from entering the interior of the tube (e.g., a nucleated eukaryotic cell, such as a nucleated human cell or a diploid human cell). The internal diameter, internal width, and/or internal height of a tube may be about 5 nm to about 4 μm, such as about 5 nm to about 3 μm, about 5 nm to about 2 μm, about 5 nm to about 1 μm, about 5 nm to about 900 nm, about 5 nm to about 800 nm, about 5 nm to about 700 nm, about 5 nm to about 600 nm, about 5 nm to about 500 nm, about 5 nm to about 400 nm, about 5 nm to about 300 nm, about 5 nm to about 200 nm, or about 5 nm to about 100 nm. The internal diameter, internal width, and/or internal height of a tube may be about 20 nm to about 4 μm, such as about 20 nm to about 3 μm, about 20 nm to about 2 μm, about 20 nm to about 1 μm, about 20 nm to about 900 nm, about 20 nm to about 800 nm, about 20 nm to about 700 nm, about 20 nm to about 600 nm, about 20 nm to about 500 nm, about 20 nm to about 400 nm, about 20 nm to about 300 nm, about 20 nm to about 200 nm, or about 20 nm to about 100 nm. The internal diameter, internal width, and/or internal height of a tube may be about 40 nm to about 4 μm, such as about 40 nm to about 3 μm, about 40 nm to about 2 μm, about 40 nm to about 1 μm, about 40 nm to about 900 nm, about 40 nm to about 800 nm, about 40 nm to about 700 nm, about 40 nm to about 600 nm, about 40 nm to about 500 nm, about 40 nm to about 400 nm, about 40 nm to about 300 nm, about 40 nm to about 200 nm, or about 40 nm to about 100 nm.

In certain preferred embodiments, the particle comprises a plurality of tubes. Each tube of the plurality of tubes may be substantially parallel. In some embodiments, at least two tubes of the plurality of tubes are not parallel. In some embodiments, none of the tubes of the plurality of tubes are parallel. The tubes may be arranged in a configuration other than parallel to distribute the openings to the tubes over different faces of the particle or to allow the particle to tumble in flow (e.g., laminar flow or turbulent flow).

A plurality of tubes may be arranged in a lattice or bundle.

A plurality of tubes may be arranged in a polyhedron, such as a regular polyhedron. The plurality of tubes may be arranged in a tetrahedron, such as a regular tetrahedron. The plurality of tubes may be arranged in a hexahedron, such as a cuboid, rectangular cuboid, or cube. The plurality of tubes may be arranged in an octahedron, such as a regular octahedron. The plurality of tubes may be arranged in a dodecahedron, such as a regular dodecahedron. The plurality of tubes may be arranged in an icosahedron, such as a regular icosahedron. In some embodiments, each edge of the polyhedron is defined by a single tube. In some embodiments, less than each edge of the polyhedron is defined by a single tube (e.g., when each of the tubes are substantially parallel).

A plurality of tubes may be arranged in a pyramid, such as a triangular pyramid, rhombic pyramid, rectangular pyramid, square pyramid, pentagonal pyramid, hexagonal pyramid, heptagonal pyramid, or octagonal pyramid. The plurality of tubes may be arranged in a right pyramid or an oblique pyramid. In some embodiments, each edge of the pyramid is defined by a single tube. In some embodiments, less than each edge of the pyramid is defined by a single tube (e.g., when each of the tubes are substantially parallel).

A plurality of tubes may be arranged in a prism, such as a triangular prism, rectangular prism, square prism, pentagonal prism, hexagonal prism, heptagonal prism, or octagonal prism. The plurality of tubes may be arranged in a right prism, an oblique prism, or a truncated prism. In some embodiments, each edge of the prism is defined by a single tube. In some embodiments, less than each edge of the prism is defined by a single tube (e.g., when each of the tubes are substantially parallel).

A plurality of tubes may be arranged in a configuration that has a length, width, and height, wherein no single dimension is more than 5 times larger than any other dimension. For example, the plurality of tubes may be arranged in a configuration wherein no single dimension is more than 4 times larger than any other dimension or no single dimension is more than 3 times larger than any other dimension. Such configurations are favorable, for example, for intravenous administration of a particle because oblong particles may not flow as well in a patient's bloodstream.

A plurality of tubes may be arranged in a configuration that has a length and diameter, wherein the length of the configuration is not more than 5 times its diameter. For the plurality of tubes may be arranged in a configuration wherein the length of the configuration is not more than 4 times its diameter or the length of the configuration is not more than 3 times its diameter. Such configurations are favorable, for example, for intravenous administration of the particle because oblong particles may not flow as well in a patient's bloodstream.

A particle may comprise 1 to 500 tubes, such as 1 to 100 tubes. A particle may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 330, 31, 32, 33, 34, 35, 36, 37, 38, 39, 40, 41, 42, 43, 44, 45, 46, 47, 48, 49, 50, 51, 52, 53, 54, 55, 56, 57, 58, 59, 50, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 tubes.

A plurality of tubes may comprise 1 to 500 tubes, such as 1 to 100 tubes. A plurality of tubes may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 330, 31, 32, 33, 34, 35, 36, 37, 38, 39, 40, 41, 42, 43, 44, 45, 46, 47, 48, 49, 50, 51, 52, 53, 54, 55, 56, 57, 58, 59, 50, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 tubes.

Each tube of the plurality of tubes may have the same length, or different tubes of the plurality of tubes may have different lengths. The average length of a tube may be about 5 nm to about 5 μm, such as about 5 nm to about 4 μm, about 5 nm to about 3 μm, about 5 nm to about 2 μm, or about 5 nm to about 1 μm. The average length of a tube may be about 50 nm to about 5 μm, such as about 50 nm to about 4 μm, about 50 nm to about 3 μm, about 50 nm to about 2 μm, or about 50 nm to about 1 μm. The average length of a tube may be about 100 nm to about 5 μm, such as about 100 nm to about 4 μm, about 100 nm to about 3 μm, about 100 nm to about 2 μm, or about 100 nm to about 1 μm. The average length of a tube may be about 300 nm to about 5 μm, such as about 300 nm to about 4 μm, about 300 nm to about 3 μm, about 300 nm to about 2 μm, or about 300 nm to about 1 μm. The average length of a tube may be about 500 nm to about 5 μm, such as about 500 nm to about 4 μm, about 500 nm to about 3 μm, about 500 nm to about 2 μm, or about 500 nm to about 1 μm.

Each tube of the plurality of tubes may have the same diameter, width, and/or height, or different tubes of the plurality of tubes may have different diameters, widths, and/or heights. The average diameter, width, and/or height of a tube may be about 5 nm to about 5 μm, such as about 5 nm to about 4 μm, about 5 nm to about 3 μm, about 5 nm to about 2 μm, about 5 nm to about 1 μm, about 5 nm to about 900 nm, about 5 nm to about 800 nm, about 5 nm to about 700 nm, about 5 nm to about 600 nm, about 5 nm to about 500 nm, about 5 nm to about 400 nm, about 5 nm to about 300 nm, about 5 nm to about 200 nm, or about 5 nm to about 100 nm. The average diameter, width, and/or height of a tube may be about 50 nm to about 5 μm, such as about 50 nm to about 4 μm, about 50 nm to about 3 μm, about 50 nm to about 2 μm, about 50 nm to about 1 μm, about 50 nm to about 900 nm, about 50 nm to about 800 nm, about 50 nm to about 700 nm, about 50 nm to about 600 nm, about 50 nm to about 500 nm, about 50 nm to about 400 nm, about 50 nm to about 300 nm, about 50 nm to about 200 nm, or about 50 nm to about 100 nm.

Each tube of the plurality of tubes may have the same internal diameter, internal width, and/or internal height, or different tubes of the plurality of tubes may have different internal diameters, widths, and/or heights. The average internal diameter, internal width, and/or internal height of a tube may be about 5 nm to about 4 μm, such as about 5 nm to about 3 μm, about 5 nm to about 2 μm, about 5 nm to about 1 μm, about 5 nm to about 900 nm, about 5 nm to about 800 nm, about 5 nm to about 700 nm, about 5 nm to about 600 nm, about 5 nm to about 500 nm, about 5 nm to about 400 nm, about 5 nm to about 300 nm, about 5 nm to about 200 nm, or about 5 nm to about 100 nm. The average internal diameter, internal width, and/or internal height of a tube may be about 20 nm to about 4 μm, such as about 20 nm to about 3 μm, about 20 nm to about 2 μm, about 20 nm to about 1 μm, about 20 nm to about 900 nm, about 20 nm to about 800 nm, about 20 nm to about 700 nm, about 20 nm to about 600 nm, about 20 nm to about 500 nm, about 20 nm to about 400 nm, about 20 nm to about 300 nm, about 20 nm to about 200 nm, or about 20 nm to about 100 nm. The average internal diameter, internal width, and/or internal height of a tube may be about 40 nm to about 4 μm, such as about 40 nm to about 3 μm, about 40 nm to about 2 μm, about 40 nm to about 1 μm, about 40 nm to about 900 nm, about 40 nm to about 800 nm, about 40 nm to about 700 nm, about 40 nm to about 600 nm, about 40 nm to about 500 nm, about 40 nm to about 400 nm, about 40 nm to about 300 nm, about 40 nm to about 200 nm, or about 40 nm to about 100 nm.

A tube may comprise, for example, a polymer. The polymer may be a naturally-occurring polymer or a synthetic polymer. The polymer may be, for example, a nucleic acid (e.g., DNA) or protein.

### V. Particles Comprising a DNA Scaffold

In some embodiments, the particle comprises a DNA scaffold, e.g., the particle may comprise a DNA origami scaffold (see, e.g., U.S. Pat. Nos. 8,554,489 and 7,842,793; U.S. Patent Application Publication Nos. 2013/0224859 and 2010/0216978; and PCT Patent Application Publication No. 2014/170898, each of which is hereby incorporated by reference).

The particle may comprise a DNA scaffold, and the DNA scaffold may comprise at least one tube or a plurality of tubes as described herein. For example, the DNA scaffold may comprise at least one substantially hexagonal tube (see, e.g., U.S. Patent Application Publication No. 2013/0224859, hereby incorporated by reference).

The DNA scaffold may comprise a honeycomb or lattice, such as a hexagonal lattice or a square lattice (see, e.g., U.S. Pat. No. 8,554,489, hereby incorporated by reference).

In some embodiments, the particle comprises a DNA scaffold, and the DNA scaffold does not comprise a tube. For example, the DNA scaffold may comprise a three-dimensional shape, such as a polyhedron, and the agent may be immobilized in the interior surface of the shape.

The DNA scaffold may comprise a polyhedron, such as a regular polyhedron. The DNA scaffold may comprise a tetrahedron, such as a regular tetrahedron. The DNA scaffold may comprise a hexahedron, such as a cuboid, rectangular cuboid, or cube. The DNA scaffold may comprise an octahedron, such as a regular octahedron. The DNA scaffold may comprise a dodecahedron, such as a regular dodecahedron. The DNA scaffold may comprise an icosahedron, such as a regular icosahedron.

The DNA scaffold may comprise a pyramid, such as a triangular pyramid, rhombic pyramid, rectangular pyramid, square pyramid, pentagonal pyramid, hexagonal pyramid, heptagonal pyramid, or octagonal pyramid. The DNA scaffold may comprise a right pyramid or an oblique pyramid.

The DNA scaffold may comprise a prism, such as a triangular prism, rectangular prism, square prism, pentagonal prism, hexagonal prism, heptagonal prism, or octagonal prism. The DNA scaffold may comprise a right prism, an oblique prism, or a truncated prism.

The DNA scaffold may comprise a length, width, and height, wherein no single dimension is more than 5 times larger than any other dimension. For example, no single dimension may be more than 4 times larger than any other dimension or no single dimension may be more than 3 times larger than any other dimension. Such configurations are favorable, for example, for intravenous administration of the particle because oblong particles may not flow as well in a patient's bloodstream.

In some embodiments, the agent is immobilized on the DNA scaffold. In some embodiments, the agent is bound to a nucleic acid comprising a nucleotide sequence that is complementary to a nucleotide sequence on the DNA scaffold, i.e., the nucleotide sequence has at least about 95%, 96%, 97%, 98%, 99%, or 100% sequence identity with the reverse complement of the nucleotide sequence of the DNA scaffold. Thus, the agent may be immobilized on a surface of the particle by hybridizing the nucleic acid to the DNA scaffold.

### VI. Particles Comprising a Shield

A particle may comprise a core subparticle and a shield, e.g., wherein the shield inhibits biomolecules bound to the core subparticle from interacting with molecules on the surface of a cell. The shield may comprise a plurality of shield components. The core subparticle may comprise silica. For example, the core subparticle may comprise a silica surface. The core subparticle may comprise gold, silicon, or a polymer. For example, the core subparticle may comprise a gold, silicon, or polymer surface.

A particle comprising an inner core subparticle and having a shield comprising a plurality of shield components attached to the core subparticle may comprise a core subparticle comprising a silica surface, such as a solid silica subparticle, a porous silica subparticle, or a silica nanoshell having a non-silica interior. The core subparticle may comprise a non-silica core material, such as silicon or gold, coated with silica. The shield components may be in the form of shield subparticles that are smaller than the core subparticle, such as nanospheres, and may comprise silica or a different material, such as gold or a polymer. The material of the surface of the core subparticle and of the shield components may be selected to be different to allow different coupling chemistry to be used to couple further components or species to the surfaces. The core subparticle may comprise a surface moiety having a reactive group, and the shield components may comprise a functional group capable of reaction with the reactive group to form a covalent bond between the surface of the core subparticles and the surface of the shield components or subparticles, as described herein.

An agent may be provided on the surface of the core subparticle but to a lesser extent, or preferably not at all, on the surface of the shield components. For example, an agent may be attached to the surface of a silica core subparticle by a bond (e.g., an ionic, covalent, or electrostatic interaction) that forms preferentially (or exclusively) with the silica core subparticle and not with the shield subparticles, e.g., having a gold surface instead of a silica surface.

In some embodiments, such a particle may comprise a silica core, such as a substantially spherical silica core, and a shield comprising a plurality of gold nanoparticles on the surface of the silica core, the gold nanoparticles having a cross-sectional dimension smaller than a cross-sectional dimension of the core, such as the diameter of the core. The gold nanoparticles may be substantially spherical. The core subparticle may be solid and non-porous or may have a porous surface. Formation of the silica core and of gold nanoparticles on the core may be achieved, for example, as described in U.S. Pat. No. 6,344,272, Sadtler and Wei, Chem. Comm. 1604-5 (2002); Meuhlig et al., ACS Nano, 5(8):6586-6592 (2011) (each of which is hereby incorporated by reference herein in its entirety). For example, gold nanoparticles may be adsorbed onto an amine-coated silica core by means of electrostatic attraction, or may be linked to a silica core having thiol groups conjugated to the silica surface that then bond to the gold surface of the gold nanoparticles.

A linker group may be provided between the silica of a core subparticle comprising silica and thiol groups for attaching a shield component to the core subparticle. The linker may have a length selected to set a maximum distance between a silica surface and a thiol group (or, when the thiol is attached to a gold surface, between the silica surface and the gold surface). In this way, the distance between the surface of the silica subparticle and the gold subparticle can be varied over a range of distances, potentially allowing a greater number of linkages (e.g., because more gold subparticles can be packed at a greater distance from the core silica subparticle), and/or strengthen the association between the silica and gold subparticles (e.g., because at shorter distances, more linkages from the surface of the silica subparticle may be able to interact with the same gold subparticle, reinforcing the association). A linker may comprise an alkylene chain whose length can be selected to vary the distance between the surface of the core subparticle and a shield subparticle.

The core subparticle may have a cross-sectional dimension, such as the diameter of a spherical or cylindrical subparticle, of 50 nm to 4 μm, such as 50 nm to 200 nm, 100 nm to 500 nm, 200 nm to 1 μm, or 500 nm to 4 μm.

Particles may be assembled from a range of core subparticle diameters and shield subparticle diameters. The available surface area of the core subparticle for scavenging of a biomolecule may depend on the diameter of the shield subparticles and the effective height above the surface of the core subparticle needed for binding of the target/agent complex to the surface, including the effective extent above the surface of any linker between the surface and the capture agent.

The number of agents that may be bound to a core subparticle may be calculated based on the surface area of the subparticle. Analogously, the number of target biomolecules that may be bound to the core subparticle may be calculated in a similar fashion. Such calculations may be confirmed, for example, by in vitro studies of protein binding, and may be used to predict the dose of particles that may be needed to scavenge a selected number of target biomolecules (or, in some embodiments, the effective dose of particles or of a formulation containing them for removing a number or reducing a concentration of target biomolecules from a system such as an in vitro system or from the circulation of a patient in treatment of disease).

A particle may comprise an available surface area for the capture of a target of 0.01 μM² to 50 μm², such as 0.01 μm² to 0.1 μM², 0.05 μm² to 0.5 μm², 0.1 μM² to 1.0 μM², 0.5 μM² to 5 μm², 1.0 μM² to 10 μM², 5 μm² to 25 μm², or 10 μm² to 50 μm². For a selected loading of agent per unit area of a core subparticle surface, a maximum dose of particles may be established as suitable to scavenge a desired quantity of target biomolecules based on the core and shield subparticle diameters.

A cross-sectional dimension, such as the diameter, of the shield subparticle may be a multiple of a cross-sectional dimension, such as the diameter, of the core particle. The multiple may be, for example, 0.01 to 0.5, such as 0.02 to 0.2, such as 0.05 to 0.1.

For effective access of a target biomolecule to an agent, the target must be able to diffuse between the shield components to reach the agent on the surface of the core subparticle. For example, targets of less than 100 kDa (e.g., sTNF-R1/2) have sizes that may readily diffuse between shielding spheres that are 40 nm in diameter or greater. For smaller shielding spheres, the effective pore length between the spheres is short, and thus shielding spheres that are smaller than 40 nm are similarly unlikely to impede diffusion.

### VII. Particles Comprising Subparticles

In some embodiments, a particle may comprise a core subparticle and a plurality of protecting subparticles. The particle may comprise a shield and the shield may comprise the plurality of protecting subparticles. The agent may be immobilized on a surface of a core subparticle, e.g., wherein the surface of a core subparticle is an inner surface. The plurality of protecting subparticles may be configured to inhibit an interaction of a biomolecule with a second member of a specific binding pair, e.g., when the biomolecule is bound to the particle. The plurality of protecting subparticles may be configured to inhibit an interaction between a biomolecule and a cell, such as a mammalian cell, e.g., when the biomolecule is bound to the particle.

The protecting subparticles may define an outer surface. In preferred embodiments, the agent is not immobilized on the surface of the protecting subparticles.

A core subparticle is preferably large enough to bind to more than one molecule of an agent. For example, a core subparticle may be about 20 nm to about 4 μm in size, such as about 50 nm to about 2 μm in size. A core subparticle may be about 100 nm to about 1000 nm, about 100 nm to about 800 nm, about 100 nm to about 600 nm, about 100 nm to about 400 nm, about 100 nm to about 200 nm, about 200 nm to about 1000 nm, about 200 nm to about 800 nm, about 200 nm to about 600 nm, about 200 nm to about 400 nm, about 400 nm to about 1000 nm, about 400 nm to about 800 nm, about 400 nm to about 600 nm, about 600 nm to about 1000 nm, or about 600 nm to about 800 nm in size. A core subparticle may be about 100 nm to about 4 μm, 100 nm to about 3 μm, 100 nm to about 2 about 200 nm to about 4 μm, 200 nm to about 3 μm, 200 nm to about 2 about 400 nm to about 4 μm, 400 nm to about 3 μm, 400 nm to about 2 about 600 nm to about 4 μm, 600 nm to about 3 μm, 600 nm to about 2 about 800 nm to about 4 μm, 800 nm to about 3 or 800 nm to about 2 μm in size.

A core subparticle may comprise metal, gold, alumina, glass, silica, silicon, starch, agarose, latex, plastic, polyacrylamide, methacrylate, a polymer, or a nucleic acid. In some embodiments, a core subparticle comprises silicon, such as porous silicon.

A core subparticle may be any shape (e.g., cubic, pyramidal, conic, spherical, cylindrical, disk, tetrahedral, hexahedral, octahedral, dodecahedral, or icosahedral) or a core subparticle may lack a defined shape.

A particle may comprise 1 core subparticle. For example, the core subparticle may be a particle of U.S. Pat. No. 7,368,295 or 8,920,625 (each of which is hereby incorporated by reference in its entirety), which is further bound to a plurality of protecting subparticles.

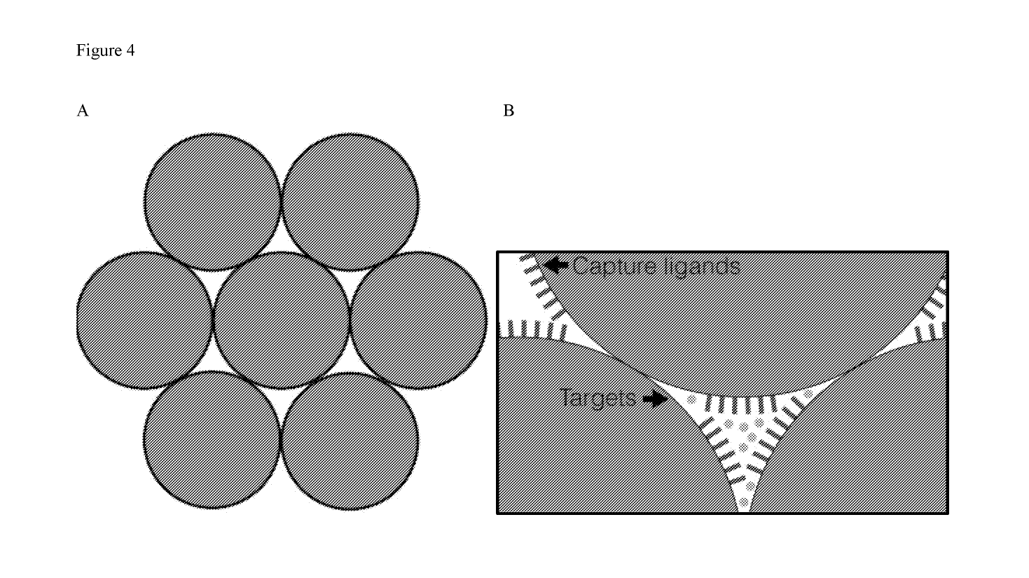

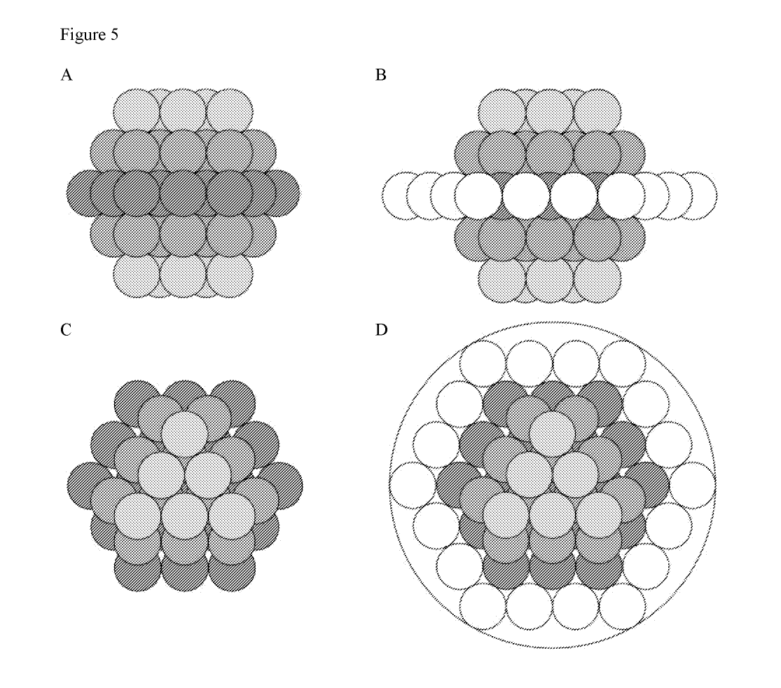

A particle may comprise a plurality of core subparticles, such as 2 to 300 core subparticles, 2 to 200 core subparticles, 2 to 150 core subparticles, 2 to 100 core subparticles, 2 to 80 core subparticles, or 2 to 42 core subparticles (see, e.g., FIGS. 4 and 5). In embodiments in which a particle comprises a plurality of core subparticles, each of the core subparticles are preferentially substantially spherical. A particle comprising a plurality of spherical core subparticles allows for voids, thereby allowing the diffusion of soluble biomolecules through the interior of the particle. Nevertheless, core subparticles of various other shapes may allow for voids. A particle comprising a plurality of core subparticles may comprise core subparticles of varying shapes and sizes.

A particle may comprise 1 to about 10⁶ core subparticles, 1 to about 10⁵ core subparticles, 1 to about 10⁴ core subparticles, 1 to about 1000 core subparticles, 1 to about 100 core subparticles, or 1 to about 10 core subparticles. A particle may comprise 2 to about 10⁶ core subparticles, 2 to about 10⁵ core subparticles, 2 to about 10⁴ core subparticles, 2 to about 1000 core subparticles, 2 to about 100 core subparticles, or 2 to about 10 core subparticles. A particle may comprise about 10 to about 10⁶ core subparticles, about 10 to about 10⁵ core subparticles, about 10 to about 10⁴ core subparticles, about 10 to about 1000 core subparticles, or about 10 to about 100 core subparticles.

The core subparticles of a plurality of core subparticles may be connected by linkers (e.g., covalent linkers). For example, each core subparticle of a plurality of core subparticles may be connected to another core subparticle by a linker.

A core subparticle may comprise pores, i.e., a core subparticle may be porous.

A protecting subparticle may comprise metal, gold, alumina, glass, silica, silicon, starch, agarose, latex, plastic, polyacrylamide, methacrylate, a polymer, or a nucleic acid. Some protecting subparticles are preferentially tethered to core subparticles by a linker, such as a covalent linker. Nevertheless, the protecting subparticles may be associated with one or more core subparticles without any covalent attachment. The protecting subparticles may be tethered to other protecting subparticles by linkers, such as by covalent linkers. For example, the protecting subparticles may form a web or net around the core subparticles, thereby sequestering the core subparticles within the particle.

In some embodiments, each protecting subparticle of the plurality of protecting subparticles are tethered to a core subparticle by a linker, such as a covalent linker. In some embodiments, some protecting subparticles of the plurality of protecting subparticles are tethered to a core subparticle, and each protecting subparticle of the plurality that is not directly tethered to a core subparticle is tethered to a protecting subparticle, i.e., such that each protecting subparticle of the plurality is either directly or indirectly tethered to a core subparticle. Thus, a particle may comprise a single layer of protecting subparticles (e.g., wherein substantially all of the protecting subparticles are directly tethered to one or more core subparticle(s)) or a particle may comprise more than one layer of protecting subparticles (e.g., wherein a substantial portion of the protecting subparticles are indirectly tethered to one or more core subparticle(s) through direct linkages with other protecting subparticles).

In some embodiments, a particle comprises a first layer of protecting subparticles comprising a first material and a second layer of protecting subparticles comprising a second material. For example, the first material may comprise silica or silicon and the second material may comprise gold. A particle may be assembled, for example, by linking the subparticles of the first layer of subparticles to one or more core subparticles and then linking the subparticles of the second layer of subparticles to the first layer of subparticles. The subparticles of the second layer may comprise a similar surface as the core subparticle(s), e.g., thereby allowing the subparticles of the first layer to link to both the core subparticle(s) and the subparticles of the second layer using similar chemistries.

A particle may be assembled using a layer-by-layer method. For example, a particle may be formed by first linking a plurality of core subparticles. The plurality of core subparticles may be substantially homogenous, e.g., such that a linking molecule may cross-link the core subparticles. The plurality of subparticles may comprise at least two types of subparticles, e.g., with different shapes, sizes, and/or surfaces that allow for a desired feature, such as voids, within the particle. After linking the plurality of core subparticles, a plurality of protecting subparticles may be linked to the plurality of core subparticles. After linking the plurality of protecting subparticles to the core subparticles, a second plurality of protecting subparticles may be linked to the plurality of protecting subparticles. Nevertheless, a particle may be assembled in many different ways, and many different layer-by-layer strategies may be employed depending on the desired properties of the particle and the desired chemistries utilized to link the subparticles.

Methods for crosslinking subparticles are known, including methods for crosslinking subparticles that comprise antibodies for use in vivo (see, e.g., Cheng, K. et al., ACS Appl Mater Interfaces 2(9):2489-2495 (2010), hereby incorporated by reference in its entirety). Such methods may be adapted to produce a particle as described herein, for example, by simply altering the relative sizes of the subparticles.

A protecting subparticle may be about 10 nm to about 4 μm in size, such as about 10 nm to about 1 μm in size, or about 20 nm to about 500 nm in size. A protecting subparticle may be about 10 nm to about 200 nm, 10 nm to about 100 nm, about 10 nm to about 80 nm, about 10 nm to about 60 nm, about 10 nm to about 40 nm, about 10 nm to about 20 nm, 20 nm to about 200 nm, about 20 nm to about 100 nm, about 20 nm to about 80 nm, about 20 nm to about 60 nm, about 20 nm to about 40 nm, 30 nm to about 200 nm, about 40 nm to about 100 nm, about 40 nm to about 80 nm, about 40 nm to about 60 nm, 60 nm to about 200 nm, about 60 nm to about 100 nm, or about 60 nm to about 80 nm in size. A protecting subparticle may be about 100 nm to about 1000 nm, about 100 nm to about 800 nm, about 100 nm to about 600 nm, about 100 nm to about 400 nm, about 100 nm to about 200 nm, about 200 nm to about 1000 nm, about 200 nm to about 800 nm, about 200 nm to about 600 nm, about 200 nm to about 400 nm, about 400 nm to about 1000 nm, about 400 nm to about 800 nm, about 400 nm to about 600 nm, about 600 nm to about 1000 nm, or about 600 nm to about 800 nm in size. A protecting subparticle may be about 100 nm to about 4 about 100 nm to about 3 about 100 nm to about 2 about 200 nm to about 4 about 200 nm to about 3 about 200 nm to about 2 about 400 nm to about 4 about 400 nm to about 3 about 400 nm to about 2 about 600 nm to about 4 about 600 nm to about 3 about 600 nm to about 2 about 800 nm to about 4 about 800 nm to about 3 or about 800 nm to about 2 μm in size.

A particle may comprise 1 to about 10⁶ protecting subparticles, about 4 to about 10⁶ protecting subparticles, about 10 to about 10⁶ protecting subparticles, 1 to about 10⁵ protecting subparticles, about 4 to about 10⁵ protecting subparticles, about 10 to about 10⁵ protecting subparticles, 1 to about 10⁴ protecting subparticles, about 4 to about 10⁴ protecting subparticles, about 10 to about 10⁴ protecting subparticles, 1 to about 1000 protecting subparticles, about 4 to about 1000 protecting subparticles, about 10 to about 1000 protecting subparticles, 1 to about 100 protecting subparticles, about 4 to about 100 protecting subparticles, or about 10 to about 100 protecting subparticles.

A core subparticle and a protecting subparticle may or may not have similar or identical shapes, sizes, and compositions. Nevertheless, a core subparticle varies from a protecting subparticle because (1) agent may be immobilized on a core subparticle whereas agent is preferentially not immobilized on a protecting subparticle, and (2) core subparticles are preferentially located in the interior of a particle whereas protecting subparticles may exist on the outer surface of a particle.

### VIII. Substantially 2-Dimensional Particles

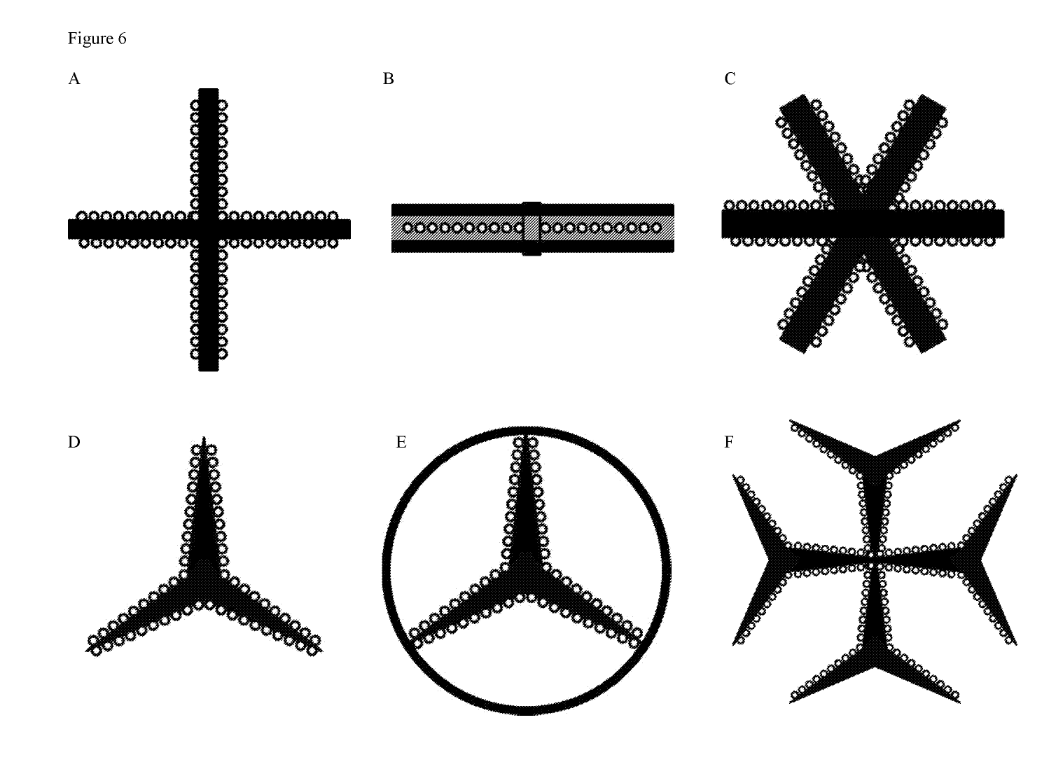

A particle may be a 2-dimensional shape. For example, a particle may be a circle, ring, cross, fishbone, ellipse, triangle, square, pentagon, hexagon, heptagon, octagon, or star. A particle may be a star and the star may be a concave hexagon, concave octagon, concave decagon, or concave dodecagon. The shape may be a regular shape or an irregular shape. Examples of substantially 2-dimensional particles are shown in FIG. 6. In some embodiments, a particle comprises a first side, a second side, and an edge.

The first side and second side may be substantially the same shape. The first side and second side may comprise a length and a width. The edge may define a height, which is the distance between the first side and the second side. The width and length may be at least 4 times larger than the height, such as 4 to 1000 times larger, 6 to 100 times larger, 8 to 75 times larger, or 10 to 50 times larger than the height. The width and/or length may be 0.2 times to about 20 times larger than the height.

An edge may comprise one or more concave or re-entrant portions. The agent may be bound to the concave or re-entrant portions of the edge. A re-entrant portion is one in which the perimeter of the particle comprises two adjacent perimeter portions at an exterior angle between them of greater than 270 degrees, such as either side of the points of a star. In this way, the capture agent may be shielded from contact with the membrane of a cell in contact with the particle.

In some embodiments, the first side and/or second side are substantially planar. In some embodiments, the first side and/or second side comprise a concave or re-entrant portion.

In some embodiments, the particle is in the form of a substantially flat star, e.g., with re-entrant portions between the points. A star may have 3, 4, 5, 6, 7, 8, 9, 10, 11, 12, 13, 14, 15, 16, or more points. The particle may comprise regular sides or irregular sides.

In some embodiments, the particle is in the form of a cross or fishbone shape, e.g., comprising a backbone with arms extending on each side outwards from the backbone to define re-entrant surface portions between the arms. The arms of a cross or fishbone may further comprise lateral projections.

The re-entrant edges between the points of the star or the arms of the cross or fishbone preferably extend a distance from the line joining the points such that a cell membrane cannot deform between the points so as to come into contact with the edges. For example, the number of points and the angle between them may determine the depth of the re-entrant edge portions between the points.

Particles suitable for use in the invention may be formed by nanofabrication, for example by nanoprinting or nanomoulding. For example, particles may be produced by the PRINT (“Particle Replication In Non-wetting Templates”) process (see, e.g., International patent application WO2007/024323; Perry, J. L. et al., Acc Chem Res. 44(10):990-998 (2011), each of which is hereby incorporated by reference). Particles may be produced by photolithography using known methods.

In some embodiments, an agent may be immobilized on the edge of a particle and not immobilized, or immobilized to a lesser extent, on the first and second sides of a particle.

In some embodiments a desirable surface area per particle is in the range 0.2 to 25 μm². The areas of the shielded edge portions of particles able to be fabricated by nanomoulding are therefore in a desirable range.

### IX. Agent

In some embodiments, the agent immobilized on the surface of a particle is a small molecule, a macrocycle compound, a polypeptide, a peptidomimetic compound, an aptamer, a nucleic acid, or a nucleic acid analog. “Small molecule” as used herein, is meant to refer to an agent, which has a molecular weight of less than about 6 kDa and most preferably less than about 2.5 kDa. Many pharmaceutical companies have extensive libraries of chemical and/or biological mixtures comprising arrays of small molecules, often fungal, bacterial, or algal extracts, which can be screened with any of the assays of the application. This application contemplates using, among other things, small chemical libraries, peptide libraries, or collections of natural products. Tan et al. described a library with over two million synthetic compounds that is compatible with miniaturized cell-based assays (J Am Chem Soc 120:8565-8566(1998)).

Peptidomimetics can be compounds in which at least a portion of a subject polypeptide is modified, and the three dimensional structure of the peptidomimetic remains substantially the same as that of the subject polypeptide. Peptidomimetics may be analogues of a subject polypeptide of the disclosure that are, themselves, polypeptides containing one or more substitutions or other modifications within the subject polypeptide sequence. Alternatively, at least a portion of the subject polypeptide sequence may be replaced with a non-peptide structure, such that the three-dimensional structure of the subject polypeptide is substantially retained. In other words, one, two or three amino acid residues within the subject polypeptide sequence may be replaced by a non-peptide structure. In addition, other peptide portions of the subject polypeptide may, but need not, be replaced with a non-peptide structure. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of humans or animals. It should be noted that peptidomimetics may or may not have similar two-dimensional chemical structures, but share common three-dimensional structural features and geometry. Each peptidomimetic may further have one or more unique additional binding elements.

Aptamers are short oligonucleotide sequences that can be used to recognize and specifically bind almost any molecule, including cell surface proteins. The systematic evolution of ligands by exponential enrichment (SELEX) process is powerful and can be used to readily identify such aptamers. Aptamers can be made for a wide range of proteins of importance for therapy and diagnostics, such as growth factors and cell surface antigens. These oligonucleotides bind their targets with similar affinities and specificities as antibodies do (see, e.g., Ulrich (2006) Handb Exp Pharmacol 173:305-326).

The agent may be an antibody, or an antigen-binding portion thereof (i.e., an antibody fragment), wherein the antibody, or antigen-binding portion thereof, specifically binds to a target (e.g., a soluble biomolecule). The agent may comprise an antibody, or an antigen-binding portion thereof, wherein the antibody, or antigen-binding portion thereof, specifically binds to a target (e.g., a soluble biomolecule). The term “antibody” refers to whole antibodies including antibodies of different isotypes, such as IgM, IgG, IgA, IgD, and IgE antibodies. The term “antibody” includes a polyclonal antibody, a monoclonal antibody, a chimerized or chimeric antibody, a humanized antibody, a primatized antibody, a deimmunized antibody, and a fully human antibody. The antibody can be made in or derived from any of a variety of species, e.g., mammals such as humans, non-human primates (e.g., orangutan, baboons, or chimpanzees), horses, cattle, pigs, sheep, goats, dogs, cats, rabbits, guinea pigs, gerbils, hamsters, rats, and mice. The antibody can be a purified or a recombinant antibody.

The term “antibody fragment,” “biomolecule-binding fragment,” “antigen-binding portion of an antibody” and similar terms refer to a fragment of an antibody that retains the ability to bind to a target antigen. Such fragments include, e.g., a single chain antibody, a single chain Fv fragment (scFv), an Fd fragment, an Fab fragment, an Fab′ fragment, or an F(ab′)₂ fragment. An scFv fragment is a single polypeptide chain that includes both the heavy and light chain variable regions of the antibody from which the scFv is derived. In addition, intrabodies, minibodies, triabodies, and diabodies are also included in the definition of antibody and are compatible for use in the methods described herein (see, e.g., Todorovska et al., J Immunol Methods 248(1):47-66 (2001); Hudson and Kortt J Immunol Methods 231(1):177-189 (1999); Poljak Structure 2(12):1121-1123 (1994); Rondon and Marasco Annual Review of Microbiology 51:257-283 (1997), the disclosures of each of which are incorporated herein by reference in their entirety). Bispecific antibodies (including DVD-Ig antibodies) are also embraced by the term “antibody.” Bispecific antibodies are monoclonal, preferably human or humanized, antibodies that have binding specificities for at least two different antigens.

As used in herein, the term “antibody” also includes, e.g., single domain antibodies such as camelized single domain antibodies. See, e.g., Muyldermans et al., Trends Biochem Sci 26:230-235(2001); Nuttall et al., Curr Pharm Biotech 1:253-263(2000); Reichmann et al., J Immunol Meth 231:25-38(1999); PCT application publication nos. WO 94/04678 and WO 94/25591; and U.S. Pat. Nos. 6,005,079, 6,015,695, and 7,794,981, all of which are incorporated herein by reference in their entireties. In some embodiments, the disclosure provides single domain antibodies comprising two VH domains with modifications such that single domain antibodies are formed.

In some embodiments, the agent is a non-antibody, scaffold protein. These proteins are, generally, obtained through combinatorial chemistry-based adaptation of pre-existing ligand- or antigen-binding proteins. For example, the binding site of human transferrin for human transferrin receptor can be modified using combinatorial chemistry to create a diverse library of transferrin variants, some of which have acquired affinity for different antigens (see Ali et al., J Biol Chem 274:24066-24073(1999)). The portion of human transferrin not involved with binding the receptor remains unchanged and serves as a scaffold, like framework regions of antibodies, to present the variant binding sites. The libraries are then screened, as an antibody library is, against a target antigen of interest to identify those variants having optimal selectivity and affinity for the target antigen. Non-antibody scaffold proteins, while similar in function to antibodies, are touted as having a number of advantages as compared to antibodies, which advantages include, among other things, enhanced solubility and tissue penetration, less costly manufacture, and ease of conjugation to other molecules of interest (see Hey et al., TRENDS Biotechnol 23(10):514-522(2005)).

One of skill in the art would appreciate that the scaffold portion of the non-antibody scaffold protein can include, e.g., all or part of: the Z domain of S. aureus protein A, human transferrin, human tenth fibronectin type III domain, kunitz domain of a human trypsin inhibitor, human CTLA-4, an ankyrin repeat protein, a human lipocalin, human crystallin, human ubiquitin, or a trypsin inhibitor from E. elaterium (see Hey et al., TRENDS Biotechnol 23(10):514-522(2005)).

In some embodiments, the agent is a natural ligand of a target biomolecule. For example, the agent can be a cytokine. As used herein, the term “cytokine” refers to any secreted polypeptide that affects the functions of cells and is a molecule which modulates interactions between cells in the immune, inflammatory or hematopoietic response. A cytokine includes, but is not limited to, monokines and lymphokines, regardless of which cells produce them. For instance, a monokine is generally referred to as being produced and secreted by a mononuclear cell, such as a macrophage and/or monocyte. Many other cells however also produce monokines, such as natural killer cells, fibroblasts, basophils, neutrophils, endothelial cells, brain astrocytes, bone marrow stromal cells, epidermal keratinocytes and B-lymphocytes. Lymphokines are generally referred to as being produced by lymphocyte cells. Examples of cytokines include, but are not limited to, Interleukin-1 (IL-1), Interleukin-2 (IL-2), Interleukin-6 (IL-6), Interleukin-8 (IL-8), Tumor Necrosis Factor-alpha (TNFα), and Tumor Necrosis Factor beta (TNFβ).

In some embodiments, the agent is a tumor necrosis factor (TNF) family ligand, e.g., the TNF family ligand is selected from TNFα, TNFβ, Fas ligand, lymphotoxin, lymphotoxin alpha, lymphotoxin beta, 4-1BB Ligand, CD30 Ligand, EDA-A1, LIGHT (TNFSF14), TNF-like ligand 1A (TL1A), TNF-related weak inducer of apoptosis (TWEAK), and TNF-related apoptosis-inducing ligand (TRAIL). The agent may be CD40 Ligand, CD27 Ligand, OX40 Ligand, B-cell activating factor (BAFF; TNFSF13B; BLYS), ectodysplasin A (EDA), activation-inducible TNFR family receptor ligand (AITRL), vascular endothelial growth inhibitor (VEGI), a proliferation-inducing ligand (APRIL), or receptor activator of nuclear factor kappa-B ligand (RANKL). In some embodiments, the target is TNFα, TNFβ, Fas ligand, lymphotoxin, lymphotoxin alpha, lymphotoxin beta, 4-1BB Ligand, CD30 Ligand, EDA-A1, LIGHT, TL1A, TWEAK, TRAIL, CD40 Ligand, CD27 Ligand, OX40 Ligand, B-cell activating factor (BAFF; TNFSF13B; BLYS), ectodysplasin A (EDA), activation-inducible TNFR family receptor ligand (AITRL), vascular endothelial growth inhibitor (VEGI), a proliferation-inducing ligand (APRIL), or receptor activator of nuclear factor kappa-B ligand (RANKL).

In some embodiments, the agent is a viral protein, or a portion thereof, which specifically binds to a target (e.g., a soluble form of a membrane protein). In some embodiments, the agent is vTNF, which is a protein capable of specifically-binding TNF that is not encoded by the genome of an organism comprising TNF and TNF receptors. vTNF includes TNF-binding proteins from viruses, such as poxvirus (e.g., Yatapoxvirus, such as Yaba-like disease virus, Tanapox virus, and Yaba monkey tumor virus; Cowpox virus; Myxoma virus; and Mousepox virus) and retrovirus (e.g., Simian foamy virus). For example, vTNF may be Crm B, Crm C, Crm D, or Crm E of the Cowpox virus, M-T2 of the Myxoma virus, S-T2 of the Simian foamy virus, vCD30 of the Cowpox virus, or TPV2L of the Tanapox virus. In some embodiments, the agent is the E6 or E7 of the human papilloma virus, which binds TNFR1, or TRAILR2 ortholog, CAR1 of the Avian sarcoma leukosis virus, which binds to TNFRs.

In some embodiments, the agent is a variant of a natural ligand for a target biomolecule, e.g., a variant interleukin polypeptide, such as variant IL-2 or variant TNFα. Variants, in accordance with some embodiments of the invention, can contain one or more amino acid substitutions, deletions, or insertions. The substitutions can be conservative or non-conservative. As used herein, the term “conservative substitution” refers to the replacement of an amino acid present in the native sequence in a given polypeptide with a naturally or non-naturally occurring amino acid having similar steric properties. Where the side-chain of the native amino acid to be replaced is either polar or hydrophobic, the conservative substitution should be with a naturally occurring amino acid or a non-naturally occurring amino acid that is also polar or hydrophobic, and, optionally, with the same or similar steric properties as the side-chain of the replaced amino acid. Conservative substitutions typically include substitutions within the following groups: glycine and alanine; valine, isoleucine, and leucine; aspartic acid and glutamic acid; asparagine, glutamine, serine, and threonine; lysine, histidine, and arginine; and phenylalanine and tyrosine. One letter amino acid abbreviations are as follows: alanine (A); arginine (R); asparagine (N); aspartic acid (D); cysteine (C); glycine (G); glutamine (Q); glutamic acid (E); histidine (H); isoleucine (I); leucine (L); lysine (K); methionine (M); phenylalanine (F); proline (P); serine (S); threonine (T); tryptophan (W), tyrosine (Y); and valine (V). Variants also include fragments of the full-length, wild-type natural ligands as well as fragments comprising one or more amino acid substitutions, insertions, or deletions relative to the wild-type, full-length natural ligand from which the fragment was derived.

The phrase “non-conservative substitutions” as used herein refers to replacement of the amino acid as present in the parent sequence by another naturally or non-naturally occurring amino acid, having different electrochemical and/or steric properties. Thus, the side chain of the substituting amino acid can be significantly larger (or smaller) than the side chain of the native amino acid being substituted and/or can have functional groups with significantly different electronic properties than the amino acid being substituted.

In some embodiments, a variant polypeptide comprises at least two (e.g., at least three, four, five, six, seven, eight, nine, 10, 15, 20, 25, 30, 35, 40, 45, 50, 55, 60, 65, 70, 75, 80, 85, 90, 95, 100, or more than 100) amino acid substitutions, deletions, or insertions, relative to the wild-type, full-length polypeptide from which it was derived. In some embodiments, a variant polypeptide comprises no more than 150 (e.g., no more than 145, 140, 135, 130, 125, 120, 115, 110, 105, 100, 95, 90, 85, 80, 75, 70, 65, 60, 55, 50, 45, 40, 35, 30, 25, 24, 23, 22, 21, 20, 19, 18, 17, 16, 15, 14, 13, 12, 11, 10, 9, 8, 7, 6, 5, 4, 3, or 2) amino acid substitutions, deletions, or insertions, relative to the wild-type, full-length polypeptide from which it was derived.

In some embodiments, a variant polypeptide (e.g., a variant IL-2 or TNFα polypeptide) retains at least 10 (e.g., at least 15, 20, 25, 30, 35, 40, 45, 50, 55, 60, 65, 70, 75, 80, 85, 90, 95, or 100) % of the ability of the wild-type, full-length polypeptide from which it was derived to bind to the target biomolecule (e.g., the member of the specific binding pair of which the wild-type, full-length polypeptide is a member). In some embodiments, the variant polypeptide will have a greater affinity for the target biomolecule than the wild-type, full-length polypeptide from which the variant was derived. For example, in some embodiments, the variant polypeptide has two (three, four, five, 10, 20, 30, 40, 50, 100, 200, 500, or even 1000) times greater affinity for the target biomolecule than does the wild-type, full-length polypeptide from which the variant polypeptide was derived. Methods for detecting or measuring the interaction between two proteins are known in the art and described above.

In some embodiments, the wild-type, full-length natural ligand modulates the activity of a cell surface receptor. Accordingly, variants of the natural ligands can have enhanced or reduced ability to modulate the activity of the receptor, relative to the activity of the wild-type natural ligand. For example, in some embodiments, a variant polypeptide has less than 90 (e.g., 85, 80, 75, 70, 65, 60, 55, 50, 45, 40, 35, 30, 25, 20, 15, 10, or less than 5) % of the ability of the full-length, wild-type polypeptide from which the variant was derived to activate a cell surface receptor protein. In some embodiments, the variant polypeptide does not activate the receptor to which it binds.

Such exemplary variant polypeptides are known in the art. For example, International Patent Application Publication No. WO 2012/085891 describes TNF family ligand variants having reduced ability to trimerize, and thus a reduced ability to activate TNF family receptors (see also U.S. Patent Application Publication No. U.S. 2014/0096274, hereby incorporated by reference). Yet the variant TNF ligands retain the ability to bind to TNF family receptors. Suitable methods for comparing activity between variant and wild-type natural ligands are known in the art.

In some embodiments, the soluble biomolecule is a ligand for a cell surface receptor, e.g., a cytokine or chemokine (e.g., MCP-1/CCL2, CCL5, CCL11, CCL12, or CCL19), such as any of those known in the art or described herein. In some embodiments, the ligand is a tumor necrosis factor (TNF) family ligand or a variant thereof. In some embodiments, the TNF family ligand is TNFα or a variant thereof. In some embodiments, the TNF family ligand is Fas ligand, lymphotoxin, lymphotoxin alpha, lymphotoxin beta, 4-1BB Ligand, CD30 Ligand, EDA-A1, LIGHT, TL1A, TWEAK, TNFβ, TRAIL, or a variant of any of the foregoing. In some embodiments, the ligand is a TGFβ superfamily ligand or variant thereof, e.g., activin A, activin B, anti-mullerian hormone, growth differentiation factor (e.g., GDF1 or GDF11), a bone morphogenic protein (BMP), inhibin (e.g., inhibin alpha, inhibin beta), lefty, persephin, nodal, neurturin, TGFβ1, TGFβ2, TGFβ3, or myostatin. In some embodiments, the ligand is hormone (e.g., a peptide hormone), such as ghrelin.

In some embodiments, the soluble biomolecule is haptoglobin or beta-2 microglobulin.

In some embodiments, the soluble biomolecule is one identified in Table 2.

TABLE 2
Exemplary Soluble Biomolecules and/or Agents

| First Member of Specific | | | | |
|---|---|---|---|---|
| Binding Pair (Soluble | Gene | Molecule | Associated | Second member of |
| Biomolecule or Agent) | Abbrev. | Class | Disease State | Specific Binding Pair |
| Tumor Necrosis Factor alpha | TNF | Cytokine | AD, obesity, | sTNF-R |
| | | | Type II | |
| | | | Diabetes | |
| | | | (T2D), | |
| | | | Alzheimer's | |
| Soluble Interleukin-2 receptor | IL2RA | Decoy | Cancer | IL-2, daclizumab, |
| | | | | basiliximab, |
| | | | | inolimomab |
| Ghrelin | GHRL | Hormone | Obesity | Ghrelin receptor |
| | | | | (GHSR1); anti-ghrelin |
| | | | | autoantibodies |
| Soluble Tumor Necrosis Factor | TNFRSF1A | Decoy | Cancer | rTNF |
| receptor-1 | | | | |
| Soluble Tumor Necrosis Factor | TNFRSF1B | Decoy | Cancer | rTNF |
| receptor-2 | | | | |
| Transforming Growth Factor | LTBP1 | Growth | Muscle | |
| beta1 | | Factor | Degeneration, | |
| | | | dis- | |
| | | | Differentiation | |
| C-C motif ligand 11, aka: | CCL11 | Cytokine | Decreased | |
| eosinophil chemotactic protein, | | | Neurogenesis | |
| eotaxin-1 | | | and Cognition | |
| Interleukin-2 | IL2 | Cytokine | AD | sIL-2R, briakinumab |
| Interleukin-6 | IL6 | Cytokine | AD | sIL-6R, olokizumab, |
| | | | | sarilumab, siltuximab |
| Interleukin-8 | CXCL8 | Cytokine | AD | IL-8R |
| Interleukin-1A | IL1A | Cytokine | AD | sIL-1RA |
| Interleukin-1B | IL1B | Cytokine | Inflammation, | canakinumab, |
| | | | diabetes | gevokizumab |
| C-X-C motif chemokine 10 | CXCL10 | Chemokine | Immune | CXCR3, |
| | | | activation | eldelumab |
| Growth Differentiation Factor | MSTN | Growth | Sarcopenia | Activin receptor |
| 8, aka: Myostatin | | Factor | | (ActRIIB) |
| Decoy receptor-3 | FAS | Decoy | Cancer | FAS-L |
| Soluble death receptor-4 | TNFRSF10A | Decoy | Cancer | TRAIL-R1 |
| Soluble death receptor-5 | TNFRSF10B | Decoy | Cancer | TRAIL-R2, |
| | | | | drozitumab |
| Fas ligand | FASLG | Cytokine | AD | sDcR3 |
| TNF-related apoptosis inducing | TNFSF10 | Cytokine | AD, T2D | sDR4/5 |
| ligand | | | | |
| Chemokine (C-X-C Motif) | CXCL1 | Chemokine | Senescence/ | |
| Ligand 1 (Melanoma Growth | | | Cancer | |
| Stimulating Activity, Alpha) | | | | |
| Amyloid beta | APP | Fragment | Alzheimer's | anti-amyloid beta |
| | | | | antibodies, e.g., |
| | | | | aducanumab |
| β2 microglobulin | B2M | Protein | Aging | |
| TNF-related weak inducer of | TNFSF12 | Cytokine | TBD | sDR3 |
| apoptosis | | | | |
| Matrix Metallopeptidase 1 | MMP1 | Protease | Senescence/ | |
| (Interstitial Collagenase) | | | Cancer | |
| Matrix Metallopeptidase 2 | MMP2 | Protease | OA/Cancer | |
| (Gelatinase A, 72kDa | | | | |
| Gelatinase, 72kDa Type IV | | | | |
| Collagenase) | | | | |
| Matrix Metallopeptidase 3 | MMP3 | Protease | Senescence/ | |
| (Stromelysin 1, Progelatinase) | | | Cancer | |
| Matrix Metallopeptidase 9 | MMP9 | Protease | OA/Cancer | |
| (Gelatinase B, 92kDa | | | | |
| Gelatinase, 92kDa Type IV | | | | |
| Collagenase) | | | | |
| Matrix Metallopeptidase 10 | MMP10 | Protease | Senescence/ | |
| (Stromelysin 2) | | | Cancer | |
| Matrix Metallopeptidase 12 | MMP12 | Protease | Senescence/ | |
| (Macrophage Elastase) | | | Cancer | |
| Indoleamine 2,3-dioxygenase | IDO1 | Enzyme | Cancer | |
| Neurogenic locus notch | NOTCH1 | Cytokine | Stem cell | |
| homolog protein 1 | | | dysfunction | |
| Neurogenic locus notch | NOTCH2 | Cytokine | Stem cell | |
| homolog protein 2 | | | dysfunction | |
| Neurogenic locus notch | NOTCH3 | Cytokine | Stem cell | |
| homolog protein 3 | | | dysfunction | |
| Neurogenic locus notch | NOTCH4 | Cytokine | Stem cell | |
| homolog protein 4 | | | dysfunction | |
| Interleukin-5 | IL5 | Cytokine | AD | Mepolizumab, |
| | | | | reslizumab |
| Soluble Interleukin-5 receptor | IL5RA | Decoy | Cancer | IL-5 |
| Soluble interleukin-6 receptor | IL6R | Decoy | Cancer | IL-6, tocilizumab |
| Soluble interleukin-8 receptor | CXCR1 | Decoy | Cancer | IL-8 |
| Soluble interleukin-1A receptor | IL1R1 | Decoy | Cancer | IL-1A |
| C-Reactive Protein | CRP | Protein | Marker of | |
| | | | inflammation | |
| Haptoglobin | HP | Protein | | |
| Fibrinogen Alpha Chain | FGA | Protein | Heart disease | |
| Soluble death receptor-3 | TNFRSF25 | Decoy | | TWEAK |
| CD47 | CD47 | Protein | Cancer | thrombospondin-1; |
| | | | | signal-regulatory |
| | | | | protein alpha (SIRPα) |

“AD” refers to autoimmune disorders and/or inflammatory disorders.
“OA” refers to osteoarthritis.

In some embodiments, an agent may bind (e.g., specifically bind) to a biomolecule selected from TNFα, TNFβ, a soluble TNF receptor, soluble TNFR-1, soluble TNFR-2, lymphotoxin, lymphotoxin alpha, lymphotoxin beta, 4-1BB Ligand, CD30 Ligand, EDA-A1, LIGHT, TL1A, TWEAK, TRAIL, soluble TRAIL receptor, IL-1, soluble IL-1 receptor, IL-1A, soluble IL-1A receptor, IL-1B, soluble IL-1B receptor, IL-2, soluble IL-2 receptor, IL-5, soluble IL-5 receptor, IL-6, soluble IL-6 receptor, IL-8, IL-10, soluble IL-10 receptor, CXCL1, CXCL8, CXCL9, CXCL10, CX3CL1, FAS ligand, soluble death receptor-3, soluble death receptor-4, soluble death receptor-5, TNF-related weak inducer of apoptosis, MMP1, MMP2, MMP3, MMP9, MMP10, MMP12, CD28, a soluble member of the B7 family, soluble CD80/B7-1, soluble CD86/B7-2, soluble CTLA4, soluble PD-L1, soluble PD-1, soluble Tim3, Tim3L, galectin 3, galectin 9, soluble CEACAM1, soluble LAG3, TGF-β, TGF-β1, TGF-β2, TGF-β3, anti-mullerian hormone, artemin, glial cell-derived neurotrophic factor (GDNF), a bone morphogenic protein (e.g., BMP2, BMP3, BMP3B, BMP4, BMP5, BMP6, BMP7, BMP8A, BMP8B, BMP10, BMP 11, BMP 12, BMP13, BMP15), a growth differentiation factor (e.g., GDF1, GDF2, GDF3, GDF3A, GDF5, GDF6, GDF7, GDF8, GDF9, GDF10, GDF11, GDF15), inhibin alpha, inhibin beta (e.g., inhibin beta A, B, C, E), lefty, nodal, neurturin, persephin, myostatin, ghrelin, sLR11, CCL2, CCL5, CCL11, CCL12, CCL19, interferon alpha, interferon beta, interferon gamma, clusterin, VEGF-A, granulocyte colony-stimulating factor (G-CSF), granulocyte-macrophage colony-stimulating factor (GM-CSF), prostaglandin E2, hepatocyte growth factor, nerve growth factor, sclerostin, complement C5, angiopoietin 2, angiopoietin 3, PCSK9, amyloid beta, activin, activin A, activin B, β2 microglobulin, soluble NOTCH1, soluble NOTCH2, soluble NOTCH3, soluble NOTCH4, soluble Jagged1, soluble Jagged2, soluble DLL1, soluble DLL3, soluble DLL4, haptoglobin, fibrinogen alpha chain, corticotropin releasing factor, corticotropin releasing factor type 1, corticotropin releasing factor type 2, urocortin 1, urocortin 2, urocortin 3, CD47, an anti-interferon γ autoantibody, an anti-interleukin 6 autoantibody, an anti-interleukin 17 autoantibody, an anti-ghrelin autoantibody, wnt, indoleamine 2,3-dioxygenase, C-reactive protein, HIV-1 gp120, endotoxin, ricin toxin, epsilon toxin of Clostridium perfringens, Staphylococcus enterotoxin B, and botulinum toxin.

In some embodiments, the agent may comprise an antibody (or an antigen-binding portion thereof) that specifically binds to TNFα, TNFβ, a soluble TNF receptor, soluble TNFR-1, soluble TNFR-2, lymphotoxin, lymphotoxin alpha, lymphotoxin beta, 4-1BB Ligand, CD30 Ligand, EDA-A1, LIGHT, TL1A, TWEAK, TRAIL, soluble TRAIL receptor, IL-1, soluble IL-1 receptor, IL-1A, soluble IL-1A receptor, IL-1B, soluble IL-1B receptor, IL-2, soluble IL-2 receptor, IL-5, soluble IL-5 receptor, IL-6, soluble IL-6 receptor, IL-8, IL-10, soluble IL-10 receptor, CXCL1, CXCL8, CXCL9, CXCL10, CX3CL1, FAS ligand, soluble death receptor-3, soluble death receptor-4, soluble death receptor-5, TNF-related weak inducer of apoptosis, MMP1, MMP2, MMP3, MMP9, MMP10, MMP12, CD28, a soluble member of the B7 family, soluble CD80/B7-1, soluble CD86/B7-2, soluble CTLA4, soluble PD-L1, soluble PD-1, soluble Tim3, Tim3L, galectin 3, galectin 9, soluble CEACAM1, soluble LAG3, TGF-β, TGF-β1, TGF-β2, TGF-β3, anti-mullerian hormone, artemin, glial cell-derived neurotrophic factor (GDNF), a bone morphogenic protein (e.g., BMP2, BMP3, BMP3B, BMP4, BMP5, BMP6, BMP7, BMP8A, BMP8B, BMP10, BMP 11, BMP 12, BMP13, BMP15), a growth differentiation factor (e.g., GDF1, GDF2, GDF3, GDF3A, GDF5, GDF6, GDF7, GDF8, GDF9, GDF10, GDF11, GDF15), inhibin alpha, inhibin beta (e.g., inhibin beta A, B, C, E), lefty, nodal, neurturin, persephin, myostatin, ghrelin, sLR11, CCL2, CCL5, CCL11, CCL12, CCL19, interferon alpha, interferon beta, interferon gamma, clusterin, VEGF-A, granulocyte colony-stimulating factor (G-CSF), granulocyte-macrophage colony-stimulating factor (GM-CSF), prostaglandin E2, hepatocyte growth factor, nerve growth factor, sclerostin, complement C5, angiopoietin 2, angiopoietin 3, PCSK9, amyloid beta, activin, activin A, activin B, β2 microglobulin, soluble NOTCH1, soluble NOTCH2, soluble NOTCH3, soluble NOTCH4, soluble Jagged1, soluble Jagged2, soluble DLL1, soluble DLL3, soluble DLL4, haptoglobin, fibrinogen alpha chain, corticotropin releasing factor, corticotropin releasing factor type 1, corticotropin releasing factor type 2, urocortin 1, urocortin 2, urocortin 3, CD47, an anti-interferon γ autoantibody, an anti-interleukin 6 autoantibody, an anti-interleukin 17 autoantibody, an anti-ghrelin autoantibody, wnt, indoleamine 2,3-dioxygenase, C-reactive protein, HIV-1 gp120, endotoxin, ricin toxin, epsilon toxin of Clostridium perfringens, Staphylococcus enterotoxin B, or botulinum toxin.

The agent may comprise ipilimumab, pembrolizumab, nivolumab, infliximab, adalimumab, certolizumab (e.g., certolizumab pegol), golimumab, etanercept, stamulumab, fresolimumab, metelimumab, demcizumab, tarextumab, brontictuzumab, mepolizumab, urelumab, canakinumab, daclizumab, belimumab, denosumab, eculizumab, tocilizumab, atlizumab, ustekinumab, palivizumab, aducanumab, bevacizumab, brolucizumab, ranibizumab, aflibercept, actoxumab, elsilimomab, siltuximab, afelimomab, nerelimomab, ozoralizumab, pateclizumab, sirukumab, omalizumab, aducanumab, bapineuzumab, crenezumab, gantenerumab, ponezumab, solanezumab, dapirolizumab, ruplizumab, toralizumab, enoticumab, alacizumab, cetuximab, futuximab, icrucumab, imgatuzumab, matuzumab, necitumumab, nimotuzumab, panitumumab, ramucirumab, zalutumumab, duligotumab, patritumab, ertumaxomab, pertuzumab, trastuzumab, alirocumab, anrukinzumab, diridavumab, drozitumab, dupilumab, dusigitumab, eculizumab, edobacomab, efungumab, eldelumab, enoblituzumab, enokizumab, evinacumab, evolocumab, exbivirumab, exbivirumab, fasinumab, felvizumab, fezakinumab, ficlatuzumab, firivumab, fletikumab, foralumab, foravirumab, fulranumab, faliximab, ganitumab, gevokizumab, fuselkumab, idarucizumab, imalumab, inolimomab, iratumumab, ixekizumab, lampalizumab, lebrikizumab, lenzilumab, lerdelimumab, lexatumumab, libivirumab, ligelizumab, lodelcizumab, lulizumab, mapatumumab, motavizumab, namilumab, nebacumab, nesvacumab, obiltoxaximab, olokizumab, orticumab, pagibaximab, palivizumab, panobacumab, pascolizumab, perakizumab, pidilizumab, pexelizumab, pritoxaximab, quilizumab, radretumab, rafivirumab, ralpancizumab, raxibacumab, regavirumab, reslizumab, rilotumumab, romosozumab, rontalizumab, sarilumab, secukinumab, setoxaximab, sevirumab, sifalimumab, siltuximab, suvizumab, tabalumab, tacatuzumab, talizumab, tanezumab, tefibazumab, TGN1412, tildrakizumab, tigatuzumab, TNX-650, tosatoxumab, tralokinumab, tremelimumab, trevogrumab, tuvirumab, urtoxazumab, vantictumab, vanucizumab, or an antigen binding portion of any one of the foregoing.

In some embodiments, the agent comprises TNFα, TNFβ, a soluble TNF receptor, soluble TNFR-1, soluble TNFR-2, vTNF, lymphotoxin, lymphotoxin alpha, lymphotoxin beta, 4-1BB Ligand, CD30 Ligand, EDA-A1, LIGHT, TL1A, TWEAK, TRAIL, soluble TRAIL receptor, IL-1, soluble IL-1 receptor, IL-1A, soluble IL-1A receptor, IL-1B, soluble IL-1B receptor, IL-2, soluble IL-2 receptor, IL-5, soluble IL-5 receptor, IL-6, soluble IL-6 receptor, IL-8, IL-10, soluble IL-10 receptor, CXCL1, CXCL8, CXCL9, CXCL10, CX3CL1, FAS ligand, soluble death receptor-3, soluble death receptor-4, soluble death receptor-5, TNF-related weak inducer of apoptosis, MMP1, MMP2, MMP3, MMP9, MMP10, MMP12, CD28, a soluble member of the B7 family, soluble CD80/B7-1, soluble CD86/B7-2, soluble CTLA4, soluble PD-L1, soluble PD-1, soluble Tim3, Tim3L, galectin 3, galectin 9, soluble CEACAM1, soluble LAG3, TGF-β, TGF-β1, TGF-β2, TGF-β3, sLR11, CCL2, CCL5, CCL11, CCL12, CCL19, activin, activin A, activin B, soluble NOTCH1, soluble NOTCH2, soluble NOTCH3, soluble NOTCH4, soluble Jagged1, soluble Jagged2, soluble DLL1, soluble DLL3, soluble DLL4, or haptoglobin.

In some embodiments, each particle comprises a plurality of agents. The plurality of agents may comprise 10 to about 10⁹ copies of the agent, such as about 10³ to about 10⁷ copies of the agent or about 10⁴ to about 10⁶ copies of the agent.

### X. Methods for Producing an Antibody

As noted above, in some embodiments the agents immobilized on the surface of the particle or particles is an antibody or antigen-binding fragment thereof. Antibodies may be elicited by methods known in the art. For example, a mammal, such as a mouse, a hamster or rabbit, may be immunized with an immunogenic form of a biomolecule (e.g., a soluble TNFR, a toxin, or a viral protein). Alternatively, immunization may occur by using a nucleic acid, which in vivo expresses a biomolecule (e.g., a soluble protein) giving rise to the immunogenic response observed. Techniques for conferring immunogenicity on a protein or peptide include conjugation to carriers or other techniques well known in the art. For instance, a peptidyl portion of a polypeptide of the invention may be administered in the presence of adjuvant. The progress of immunization may be monitored by the detection of antibody titers in plasma or serum. Standard ELISA or other immunoassays may be used with the immunogen as antigen to assess the concentrations of antibodies.

Following immunization, antisera reactive with a polypeptide of the invention may be obtained and, if desired, polyclonal antibodies isolated from the serum. To produce monoclonal antibodies, antibody producing cells (lymphocytes) may be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), as the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with the polypeptides of the invention and the monoclonal antibodies isolated.

### XI. Positioning of an Agent on a Particle

In some embodiments, the geometry of the particle is such that the immobilized agent has a reduced, or substantially reduced, ability to interact with a biomolecule on the surface of a cell, such as an immune cell, blood cell, or lymphocyte. An immobilized agent may have less than 50% (e.g., 45, 40, 35, 30, 25, 20, 15, 10, 9, 8, 7, 6, 5, 4, 3, 2, or 1%) of the ability to bind to a biomolecule on a surface of a cell relative to a free, soluble form of the agent. For example, in some embodiments, TNFα or IL-2 immobilized on the surface of a particle described herein has less than 50 (e.g., 45, 40, 35, 30, 25, 20, 15, 10, 9, 8, 7, 6, 5, 4, 3, 2, or 1) % of the ability of free TNFα or IL-2 to bind to a TNFα receptor or IL-2 receptor on the surface of a cell.

In some embodiments, the soluble biomolecule bound to the particle has a reduced, or substantially reduced, ability to interact with its cognate ligand (the second member of the specific binding pair). The biomolecule may be bound to the particle by virtue of the agent. A biomolecule bound to a particle may have less than 50% (e.g., 45, 40, 35, 30, 25, 20, 15, 10, 9, 8, 7, 6, 5, 4, 3, 2, or 1%) of the ability to interact with its cognate ligand relative to the ability of an unbound, biomolecule. For example, a soluble TNFR bound to a particle described herein has less than 50 (e.g., 45, 40, 35, 30, 25, 20, 15, 10, 9, 8, 7, 6, 5, 4, 3, 2, or 1) % of the ability of free, soluble TNFR to interact with free TNFα. In another example, a soluble virion bound to a particle described herein has less than 50 (e.g., 45, 40, 35, 30, 25, 20, 15, 10, 9, 8, 7, 6, 5, 4, 3, 2, or 1) % of the ability of free virion to interact with its cognate cell surface receptor(s) and infect a cell.

In some embodiments, the agent may be immobilized on an inner surface of a particle (e.g., the pores of a porous particle or the inner surface of a tube). In some embodiment, the agent can be immobilized on the outer surface of a particle, but is sterically precluded from interacting with a cell surface by way of one or more protrusions from the particle. In some embodiments, e.g., toroidal particles, the agent is immobilized on the inner surface of the particle such that the agent has a reduced, or substantially reduced, ability to interact with a biomolecule on the surface of a cell and/or the soluble biomolecule bound to the particle by virtue of the agent has a reduced, or substantially reduced, ability to interact with its cognate ligand (the second member of the specific binding pair).

Exemplary particle geometries capable of reducing or substantially reducing the interaction of an agent with a biomolecule on a cell surface, or the interaction between a biomolecule bound to the particle, and its cognate ligand, are set forth in FIGS. 1 to 6 and described herein.

### XII. Clearance Agents and Coatings

In some embodiments, a particle comprises a clearance agent. The clearance agent may facilitate clearance of the particle through a biological pathway, such as by excretion in the urine, degradation, excretion by a hepatobiliary pathway, and/or phagocytosis.

For example, the particle may comprise a reservoir, wherein the reservoir comprises a clearance agent. The reservoir may be a hole or void in the body of a particle, e.g., a void in the body of a porous silicon particle.

For particles comprising pores, the reservoir may be a pore or the reservoir may be larger or smaller than the average pore size. A reservoir may consist of a recess in the body of a particle (e.g., a shallow recess), wherein the width or diameter of the recess is larger than the width or diameter of the average pore size. The width or diameter of a reservoir may be at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 90, 100, 110, 120, 130, 140, 150, 175, 200, 250, 300, 400, or even about 500 times as large as the width or diameter of the average pore size.

The width or diameter of the reservoir may be about 2 times to about 10 times the width or diameter of the average pore size, such as about 2 times to about 8 times or about 2 times to about 6 times. The width or diameter of a reservoir may be about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 90, 100, 110, 120, 130, 140, 150, 175, 200, 250, 300, 400, or even about 500 times as large as the width or diameter of the average pore size.

For particles comprising a DNA scaffold, a reservoir may be an interior region of the DNA scaffold. The reservoir (e.g., interior region) may be inaccessible to cells, e.g., the DNA scaffold may be constructed such that the scaffold sterically hinders cells from entering the interior region. In some embodiments, the reservoir (e.g., interior region) is inaccessible to extracellular proteins, e.g., the DNA scaffold may be constructed such that the scaffold sterically hinders extracellular proteins from entering the reservoir. The reservoir (e.g., interior region) may be inaccessible to antibodies. Nevertheless, the DNA scaffold may allow for the reservoir (e.g., interior region) to become accessible to cells and/or extracellular proteins after a predetermined period of time. For example, the DNA scaffold may comprise a biodegradable wall that may degrade after a predetermined period of time (e.g., by hydrolysis), thereby exposing the clearance agent to cells and/or extracellular proteins. The DNA scaffold may comprise a biodegradable latch that may degrade after a predetermined period of time (e.g., by hydrolysis), allowing the DNA scaffold to undergo a conformational change, thereby exposing the clearance agent to cells and/or extracellular proteins (see, e.g., PCT Patent Application Publication No. WO2014/170899, hereby incorporated by reference). Similarly, the DNA scaffold may comprise a reservoir that comprises and opening, as described below.

The reservoir may comprise an opening. The opening may be covered by a cap or member, thereby inhibiting interactions between the clearance agent and cells and/or extracellular proteins (e.g., antibodies). The cap or member may comprise a polymer, such as a biodegradable polymer. The cap or member may degrade after a predetermined period of time (e.g., by hydrolysis), thereby exposing the clearance agent to cells and/or extracellular proteins. The cap or member may degrade (e.g., biodegrade) after exposure to a biological fluid (e.g., blood plasma or extracellular fluid) for about 1 day to about 5 years, such as about 1 day to about 4 years, about 1 day to about 3 years, or about 1 day to about 1 year.

A predetermined period of time may be a period of time that the particle is in a liquid (e.g., an aqueous liquid). The predetermined period of time may be a period of in vivo residence of a particle (e.g., exposure to biological fluids, pH, enzymes, and/or temperatures). The predetermined period of time may be determined, at least in part, by the binding of the particle to a biomolecule. For example, the particle may be configured such that the binding of a biomolecule exposes the clearance agent to cells and/or extracellular proteins (see, e.g., PCT Patent Application Publication No. WO2014/170899, hereby incorporated by reference). The predetermined period of time may be about 1 day to about 5 years, such as about 1 day to about 3 years, or about 1 day to about 1 year.

Exemplary materials suitable for use as caps or membranes, are described in U.S. Pat. No. 7,918,842, which is hereby incorporated by reference. In general, these materials degrade or dissolve either by enzymatic hydrolysis or exposure to water in vivo or in vitro, or by surface or bulk erosion. Representative synthetic, biodegradable polymers include: poly(amides) such as poly(amino acids) and poly(peptides); poly(esters) such as poly(lactic acid), poly(glycolic acid), poly(lactic-co-glycolic acid), and poly(caprolactone); poly(anhydrides); poly(orthoesters); poly(carbonates); and chemical derivatives thereof (substitutions, additions of chemical groups, for example, alkyl, alkylene, hydroxylations, oxidations, and other modifications routinely made by those skilled in the art), copolymers and mixtures thereof. Other polymers that may be used in caps or membranes include: poly(ethers) such as poly(ethylene oxide), poly(ethylene glycol), and poly(tetramethylene oxide); vinyl polymers—poly(acrylates) and poly(methacrylates) such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; poly(siloxanes); and any chemical derivatives thereof (substitutions, additions of chemical groups, for example, alkyl, alkylene, hydroxylations, oxidations, and other modifications routinely made by those skilled in the art), copolymers and mixtures thereof. In certain embodiments, the reservoir cap is formed from one or more cross-linked polymers, such as cross-linked polyvinyl alcohol.

In some embodiments, a particle comprises a coating. In some embodiments, the coating comprises a clearance agent. The coating may mask a clearance agent.

The particle may comprise a first surface and a second surface; the agent may be immobilized on the first surface; and the coating may cover at least a portion of the second surface. The first surface may be an interior surface or an inner surface, e.g., the first surface may be oriented such that the agent has a reduced ability to bind to a molecule on a cell surface. Examples of an interior surface or inner surface include the inner walls of a pore, reservoir, or tube, the inner circumferential surface of a toroid, or the hollow of a concave surface. Other examples of an interior surface or inner surface include the outer surface of a particle, wherein the outer surface is protected from interactions with cells by one or more protrusions. The second surface may be an exterior surface or outer surface, e.g., the second surface may be oriented such that the coating can interact with a cell. In some embodiments, a particle may comprise one or more core subparticles and a plurality of protecting subparticles. The particle may comprise a shield and the shield may comprise the plurality of protecting subparticles. The first surface may be the surface of the one or more core particles and the second surface may be the surface of the protecting subparticles.

A coating may inhibit interactions between particles, e.g., the coating may reduce the propensity of particles to form aggregates. The coating may inhibit interactions between a particle and cells, e.g., by presenting a biologically-inert surface. The coating may inhibit non-specific interactions with extracellular molecules, e.g., non-specific adsorption of biomolecules. A coating may inhibit specific interactions with cells or extracellular molecules, e.g., a coating may disfavor or delay the excretion or phagocytosis of a particle. A coating may target a particle for excretion or phagocytosis. A coating or other feature (e.g., an “excretion-inducing compound”) that targets a particle for excretion or phagocytosis may be masked by a coating (e.g., a second coating) that delays the excretion or phagocytosis of the particle, e.g., to promote maintenance of the particles in the bloodstream for a predetermined amount of time.

A coating may comprise a plurality of elongated coating molecules bound at one end to the surface of the particle. A coating may inhibit interactions between a biomolecule bound to a particle and a second member of the specific binding pair that includes the biomolecule. A coating may inhibit interactions between a biomolecule bound to a particle and a cell. An agent may be oriented on a particle relative to a coating such that the agent has a reduced ability to bind to a molecule on the surface of a cell. An agent may be oriented on a particle relative to a coating such that the agent has a reduced ability to bind to a target on the surface of a cell. An agent may be oriented on a particle relative to a coating such that the coating sterically inhibits the agent from binding to a molecule on the surface of a cell. An agent may be oriented on a particle such that the coating sterically inhibits the agent from binding to a target on the surface of a cell. A coating may be oriented on a particle such that the agent of the particle has a reduced ability to bind to a molecule on the surface of a cell. A coating may reduce the ability of the agent of a particle to activate a cell surface receptor protein, relative to the ability of a natural ligand of the cell surface receptor protein.

A particle may comprise a second coating, e.g., wherein the second coating consists of a second plurality of coating molecules. A particle may comprise a second plurality of coating molecules. The second coating and/or second plurality of coating molecules may decrease the clearance of the particle in vivo, e.g., by masking the coating and/or plurality of coating molecules. The second coating and/or second plurality of coating molecules may be biodegradable, e.g., to expose the coating and/or plurality of coating molecules to cells and/or extracellular proteins after a predetermined period of time. The second coating and/or second plurality of coating molecules may comprise a biodegradable polymer, e.g., each molecule of the second plurality of coating molecules may comprise a biodegradable polymer. The second coating and/or second plurality of coating molecules may comprise CD47, which inhibits phagocytosis.

In some embodiments, the particle comprises a first surface (e.g., an interior surface) and a second surface (e.g., an exterior surface or outer surface); the agent is immobilized on the first surface; and the coating covers at least a portion of the second surface. The orientation of the first surface may reduce the ability of the agent to interact with molecules on a cell surface. The orientation of the second surface may permit interactions between the coating and cells, extracellular molecules, and/or different particles. An “interaction” between the coating and cells, extracellular molecules, and/or different particles may be a weak, neutral, or unfavorable interaction, e.g., to disfavor stable binding of the particle to a cell, extracellular molecule, or other particle. Alternatively, an interaction between the coating and either cells and/or extracellular molecules may be a specific or designed interaction, e.g., to favor clearance of the particle through a biological pathway, such as phagocytosis. In certain preferred embodiments, the second surface is substantially free of agent. In certain preferred embodiments, the first surface is substantially free of coating. In certain preferred embodiments, the coating covers substantially all of the second surface.

In some embodiments, the particle comprises a first surface (e.g., an interior surface) and a second surface (e.g., an exterior surface or outer surface); the agent is immobilized on the first surface and the second surface; and the coating covers at least a portion of the second surface. In such embodiments, the coating (and/or a second coating) may inhibit interactions between the agent and molecules on a cell surface. In certain preferred embodiments, the coating covers substantially all of the second surface.

In some embodiments, the particle comprises a first surface (e.g., an interior surface) and a second surface (e.g., an exterior surface or outer surface); the agent is immobilized on the first surface; and the coating covers at least a portion of the first surface and at least a portion of the second surface. In such embodiments, the coating preferably does not affect the ability of the agent to specifically bind to a biomolecule. In certain preferred embodiments, the coating covers substantially all of the second surface.

In some embodiments, the particle comprises a surface; the agent is immobilized on the surface; and the coating covers at least a portion of the surface. In such embodiments, the coating may not affect the ability of the agent to specifically bind to a biomolecule. The coating may allow for some of the agent to specifically bind to a biomolecule and inhibit interactions between some of the agent and biomolecule. The coating may inhibit interactions between the agent and molecules on a cell surface. In certain preferred embodiments, the coating covers substantially all of the surface.

In some embodiments, the particle comprises a coating that covers at least a portion of the second surface and a second coating that covers at least a portion, such as substantially all, of the coating on the second surface. In such embodiments the coating may comprise a clearance agent, such as an “excretion-inducing compound” to target a particle for excretion or phagocytosis. Such a coating may comprise beta-cyclodextrin. The second coating may comprise a material, e.g., a second plurality of coating molecules, to inhibit interaction with cells and/or inhibit non-specific interactions with extracellular molecules, e.g., non-specific adsorption of biomolecules. The second coating may be biodegradable, e.g., to expose the coating on the second surface to cells and/or extracellular proteins after a predetermined period of time. For example, in a particle comprising one or more core subparticles and a plurality of protecting sub-particles, wherein a capture agent is immobilized on the surface on the core subparticle(s) (i.e., the first surface), at least a portion of the surface of the protecting subparticles (i.e., the second surface) comprises a coating, for example a coating comprising either a clearance agent or a coating comprising a material to inhibit interaction with cells and/or to inhibit non-specific interaction with extracellular molecules.

A coating may comprise coating molecules, e.g., a coating may consist of a plurality of coating molecules or a coating may consist of a population of coating molecules. As used herein, the terms “plurality of coating molecules” and “population of coating molecules” each refer to a coating. The term “coating,” however, may refer to additional compositions, such as a hydrogel. A coating molecule may be a clearance agent (and thus, a clearance agent may be a coating molecule).

A particle may comprise a plurality of coating molecules. The particle may comprise a surface and a plurality of agents immobilized on the surface, and at least one molecule of the plurality of coating molecules may be bound to the surface. For example, all or substantially all of the molecules of the plurality of coating molecules may be bound to the surface.

The particle may comprise a surface and a second surface, wherein a plurality of agents immobilized on the surface, and at least one molecule of the plurality of coating molecules may be bound to the second surface. For example, all or substantially all of the molecules of the plurality of coating molecules may be bound to the second surface. In some embodiments, some of the molecules of the plurality of coating molecules are bound to the surface and some of the molecules of the plurality of coating molecules are bound to the second surface.

In some embodiments, the coating molecules increase the clearance of the particle in vivo. For example, the coating molecules may comprise a pathogen-associated molecular pattern.

In some embodiments, the particles described herein have a coating comprising an excretion-inducing compound, which facilitates the removal of the particles from the circulation, e.g., via the kidneys, liver/intestines (e.g., via bile), or phagocytosis (e.g., by antigen-presenting cells). A plurality of coating molecules may be a plurality of excretion-inducing compounds. For example, in embodiments in which the particles are toroidal, the inner circumferential surface (e.g., a first surface) may comprise an immobilized agent and the outer surface (e.g., a second surface) may comprise a compound that induces the clearance of the particles, e.g., by the kidneys, liver, or macrophages. In some embodiments, the excretion-inducing compound is programmed. That is, the compound can be covered with a coating that degrades (e.g., through the action of enzymes, hydrolysis, or gradual dissolution) over time (e.g., a predetermined amount of time) eventually exposing the excretion-inducing compound or other feature that increases the rate of clearance. The coating may degrade after exposure to a biological fluid (e.g., blood plasma or extracellular fluid) for about 1 day to about 5 years, such as about 1 day to about 3 years, or about 1 day to about 1 year. Thus, the in vivo residence of a particle may be modified and/or controlled.

A coating may comprise an organic polymer, such as polyethylene glycol (PEG). An organic polymer may be attached to a particle, e.g., attached to a surface of the particle. The organic polymer may include PEG, polylactate, polylactic acids, sugars, lipids, polyglutamic acid, polyglycolic acid (PGA), polylactic acid (PLA), poly(lactic-co-glycolic acid) (PLGA), polyvinyl acetate (PVA), and combinations thereof. In certain embodiments, the particle is covalently conjugated with PEG, which discourages adsorption of serum proteins, facilitates efficient urinary excretion and decreases aggregation of the particle (see, e.g., Burns et al., Nano Letters, 9(1):442-448 (2009) and U.S. Patent Application Publication Nos. 2013/0039848 and 2014/0248210, each of which is hereby incorporated by reference).

In one embodiment, the coating comprises at least one hydrophilic moiety, for example, Pluronic® type polymers (a nonionic polyoxyethylene-polyoxypropylene block co-polymer with the general formula HO(C₂H₄O)ₐ(—C₃H₆O)_{b}(C₂H₄O)ₐH), a triblock copolymer poly(ethylene glycol-b-(DL-lactic acid-co-glycolic acid)-b-ethylene glycol) (PEG-PLGA-PEG), a diblock copolymer polycaprolactone-PEG (PCL-PEG), poly(vinylidene fluoride)-PEG (PVDF-PEG), poly(lactic acid-co-PEG) (PLA-PEG), poly(methyl methacrylate)-PEG (PMMA-PEG) and so forth. In an embodiment with such a moiety, the hydrophilic moiety is a PEG moiety such as: a [Methoxy(Polyethyleneoxy)Propyl]-Trimethoxysilane (e.g., CH₃(OC₂H₄)₆₋₉(CH₂)OSi(OCH₃)₃), a [Methoxy(Polyethyleneoxy)Propyl]-Dimethoxysilane (e.g., CH₃(OC₂H₄)₆₋₉(CH₂)OSi(OCH₃)₂), or a [Methoxy(Polyethyleneoxy)Propyl]-Monomethoxysilane (e.g., CH₃(OC₂H₄)₆₋₉(CH₂)OSi(OCH₃)). Suitable coatings are described, for example, in U.S. Patent Application Publication No. 2011/0028662 (hereby incorporated by reference).

The coating may include a polyhydroxylated polymer, such as natural polymers or hydroxyl-containing polymers including multiply-hydroxylated polymers, polysaccharides, carbohydrates, polyols, polyvinyl alcohol, poly amino acids such as polyserine, or other polymers such as 2-(hydroxyethyl)methacrylate, or combinations thereof. In some embodiments, the polyhydroxylated polymers are polysaccharides. Polysaccharides include, mannan, pullulan, maltodextrin, starches, cellulose, and cellulose derivatives, gums, xanthan gum, locust bean gum, or pectin, combinations thereof (see, e.g., U.S. Patent Application Publication No. 2013/0337070, hereby incorporated by reference).

In some embodiments, the coating comprises a zwitterionic polymer (see, e.g., U.S. Patent Application Publication Nos. 2014/0235803, 2014/0147387, 2013/0196450, and 2012/0141797; and U.S. Pat. No. 8,574,549, each of which is hereby incorporated by reference).

Other suitable coatings include poly-alpha hydroxy acids (including polyactic acid or polylactide, polyglycolic acid, or polyglycolide), poly-beta hydroxy acids (such as polyhydroxybutyrate or polyhydroxyvalerate), epoxy polymers (including polyethylene oxide (PEO)), polyvinyl alcohols, polyesters, polyorthoesters, polyamidoesters, polyesteramides, polyphosphoesters, and polyphosphoester-urethanes. Examples of degradable polyesters include: poly(hydroxyalkanoates), including poly(lactic acid) or (polylactide, PLA), poly(glycolic acid) or polyglycolide (PGA), poly(3-hydroxybutyrate), poly(4-hydroxybutyrate), poly(3-hydroxyvalerate), and poly(caprolactone), or poly(valerolactone). Examples of polyoxaesters include poly(alkylene oxalates) such as poly(ethylene oxalate)) and polyoxaesters containing amido groups. Other suitable coating materials include polyethers including polyglycols, ether-ester copolymers (copoly(ether-esters)) and polycarbonates. Examples of biodegradable polycarbonates include polyorthocarbonates, polyiminocarbonates, polyalkylcarbonates such as poly(trimethylene carbonate), poly(1,3-dioxan-2-one), poly(p-dioxanone), poly(6,6-dimethyl-1,4-dioxan-2-one), poly(1,4-dioxepan-2-one), and poly(1,5-dioxepan-2-one). Suitable biodegradable coatings can also include polyanhydrides, polyimines (such as poly(ethylene imine) (PEI)), polyamides (including poly-N-(2-hydroxypropyl)-methacrylamide), poly(amino acids) (including a polylysine such as poly-L-lysine, or a polyglutamic acid such as poly-L-glutamic acid), polyphosphazenes (such as poly(phenoxy-co-carboxylatophenoxy phosphazene), polyorganophosphazenes, polycyanoacrylates and polyalkylcyanoacrylates (including polybutylcyanoacrylate), polyisocyanates, and polyvinylpyrrolidones.

The chain length of a polymeric coating molecule may be about 1 to about 100 monomer units, such as about 4 to about 25 units.

A particle may be coated with a naturally occurring polymer, including fibrin, fibrinogen, elastin, casein, collagens, chitosan, extracellular matrix (ECM), carrageenan, chondroitin, pectin, alginate, alginic acid, albumin, dextrin, dextrans, gelatins, mannitol, n-halamine, polysaccharides, poly-1,4-glucans, starch, hydroxyethyl starch (HES), dialdehyde starch, glycogen, amylase, hydroxyethyl amylase, amylopectin, glucoso-glycans, fatty acids (and esters thereof), hyaluronic acid, protamine, polyaspartic acid, polyglutamic acid, D-mannuronic acid, L-guluronic acid, zein and other prolamines, alginic acid, guar gum, and phosphorylcholine, as well as co-polymers and derivatives thereof. The coating may also comprise a modified polysaccharide, such as cellulose, chitin, dextran, starch, hydroxyethyl starch, polygluconate, hyaluronic acid, and elatin, as well as co-polymers and derivative thereof.

A particle may be coated with a hydrogel. The hydrogel can be formed, for example, using a base polymer selected from any suitable polymer, such as poly(hydroxyalkyl (meth)acrylates), polyesters, poly(meth)acrylamides, poly(vinyl pyrrolidone), or polyvinyl alcohol. A cross-linking agent can be one or more of peroxides, sulfur, sulfur dichloride, metal oxides, selenium, tellurium, diamines, diisocyanates, alkyl phenyl disulfides, tetraalkyl thiuram disulfides, 4,4′-dithiomorpholine, p-quinine dioxime and tetrachloro-p-benzoquinone. Also, boronic acid-containing polymers can be incorporated in hydrogels, with optional photopolymerizable groups.

In certain preferred embodiments, the coating comprises a material that is approved for use by the U.S. Food and Drug Administration (FDA). These FDA-approved materials include polyglycolic acid (PGA), polylactic acid (PLA), Polyglactin 910 (comprising a 9:1 ratio of glycolide per lactide unit, and known also as VICRYL™), polyglyconate (comprising a 9:1 ratio of glycolide per trimethylene carbonate unit, and known also as MAXON™), and polydioxanone (PDS).

The attachment of a coating to a particle may be accomplished by a covalent bond or a non-covalent bond, such as by ionic bond, hydrogen bond, hydrophobic bond, coordination, adhesive, or physical absorption or interaction.

Conventional nanoparticle coating methods include dry and wet approaches. Dry methods include: (a) physical vapor deposition (Zhang, Y. et al., Solid State Commun. 115:51 (2000)), (b) plasma treatment (Shi, D. et al., Appl. Phys. Lett. 78:1243 (2001); Vollath, D. et al., J. Nanoparticle Res. 1:235 (1999)), (c) chemical vapor deposition (Takeo, O. et al., J. Mater. Chem. 8:1323 (1998)), and (d) pyrolysis of polymeric or non-polymeric organic materials for in situ precipitation of nanoparticles within a matrix (Sglavo, V. M. et al., J. Mater Sci. 28:6437 (1993)). Wet methods for coating particles include: (a) sol-gel processes and (b) emulsification and solvent evaporation techniques (Cohen, H. et al., Gene Ther. 7:1896 (2000); Hrkach, J. S. et al., Biomaterials 18:27 (1997); Wang, D. et al., J. Control. Rel. 57:9 (1999)). A coating may be applied by electroplating, spray coating, dip coating, sputtering, chemical vapor deposition, or physical vapor deposition. Additionally, methods for coating various nanoparticles with polysaccharides are known in the art (see, e.g., U.S. Pat. No. 8,685,538 and U.S. Patent Application Publication No. 2013/0323182, each of which is hereby incorporated by reference).

In some embodiments, the particles may be adapted to facilitate clearance by renal excretion. Renal clearance for subjects with normal renal function generally requires particles with at least one dimension that is less than 15 nm (see, e.g., Choi, H. S., et al., Nat Biotechnol 25(1):1165 (2007); Longmire, M. et al., Nanomedicine 3(5):703 (2008)). Nevertheless, larger particles may be excreted in the urine. For embodiments in which a particle is too large for renal clearance, the particle may nevertheless be cleared following in vivo degradation to a smaller size.

In some embodiments, the particles may be adapted to facilitate clearance by hepatobiliary excretion. The mononuclear phagocytic system (MPS), which includes the Kupffer cells in the liver, is involved in the liver uptake and subsequent biliary excretion of nanoparticles. Certain size and surface properties of nanoparticles are known to increase uptake by the MPS in the liver (see Choi et al., J. Dispersion Sci. Tech. 24(3/4):475-487 (2003); and Brannon-Peppas et al., J. Drug Delivery Sci. Tech. 14(4):257-264 (2004), each of which is incorporated by reference). For example, increasing the hydrophobicity of a particle is known to increase uptake by the MPS. Thus, one of ordinary skill in the art can select for particles having certain characteristics to modulate biliary excretion. The hepatobiliary system allows for the excretion of particles that are somewhat larger than those that may be excreted through the renal system (e.g., 10 to 20 nm). For embodiments in which a particle is too large for hepatobiliary excretion, the particle may nevertheless be cleared following in vivo degradation to a smaller size. In such embodiments, a coating that facilitates clearance by hepatobiliary excretion may cover a portion of an inner surface of a particle such that the coating becomes exposed following degradation of the particle. The particle may comprise a plurality of coating molecules, e.g., hydrophobic molecules, that cover a portion of a surface. The surface may be exposed following degradation of the particle, allowing for clearance of the degraded particle.

In some embodiments, the particle is adapted to facilitate clearance by phagocytosis. For example, the particle may comprise a clearance agent, wherein the clearance agent comprises a pathogen-associated molecular pattern, e.g., for recognition by macrophages. Pathogen-associated molecular patterns (PAMPs) include unmethylated CpG DNA (bacterial), double-stranded RNA (viral), lipopolysacharride (bacterial), peptidoglycan (bacterial), lipoarabinomannan (bacterial), zymosan (yeast), mycoplasmal lipoproteins such as MALP-2 (bacterial), flagellin (bacterial), poly(inosinic-cytidylic) acid (bacterial), lipoteichoic acid (bacterial), and imidazoquinolines (synthetic). In preferred embodiments, the PAMP clearance agent is masked such that macrophages do not engulf the particle prior to the binding of the particle to one or more targets. For example, a PAMP clearance agent may be masked by any one of the aforementioned coatings (e.g., a polymeric coating, such as a biodegradable polymeric coating). Macrophages can engulf particles as large as 20 μm (see, e.g., Cannon, G. J. and Swanson, J. A., J. Cell Science 101:907-913 (1992); Champion, J. A., et al., Pharm Res 25(8):1815-1821 (2008)). In some embodiments, a clearance agent that facilitates clearance by phagocytosis may cover a portion of an inner surface of a particle such that the clearance agent becomes exposed following degradation of the particle. The particle may comprise a plurality of clearance agents, e.g., PAMPs, that cover a portion of a surface. The surface may be exposed following degradation of the particle, allowing for clearance of the degraded particle. The clearance agent may cover a portion of a surface that overlaps a surface comprising an agent. The clearance agent (e.g., PAMPs) may elicit an immune response against the particle, e.g., following the degradation of a second coating or following the degradation of the particle.

In some embodiments, an immune response directed against a clearance agent (e.g., PAMPs) may outcompete an immune response directed against the agent and/or agent/biomolecule complex, thereby inhibiting or delaying the onset of an immune response directed against the agent and/or agent/biomolecule complex. For example, degradation of a particle may expose both a clearance agent and an agent (and/or agent/biomolecule complex) to leukocytes. A PAMP clearance agent may allow for rapid clearance of the degraded particle by macrophages, thereby delaying an immune response (e.g., B-cell mediated immune response) against the agent and/or agent/biomolecule complex.

A clearance agent may be calreticulin, which induces phagocytosis.

In certain preferred embodiments, the coating molecule comprises a nucleic acid, e.g., for hybridizing with a coating molecule to a particle comprising a DNA scaffold. For example, a particle may comprise a nucleic acid and a coating molecule, wherein the coating molecule comprises a complementary nucleic acid that can hybridize with the nucleic acid, thereby forming a bond between the coating molecule and the particle (i.e., hydrogen bonds). The nucleic acid may comprise a nucleotide sequence and the complementary nucleic acid may comprise a complementary nucleotide sequence, e.g., wherein the nucleotide sequence has at least 95%, 96%, 97%, 98%, or 99% sequence i.e. identity with the reverse complement of the complementary nucleotide sequence. The nucleotide sequence may have 100% sequence i.e. identity with the reverse complement of the complementary nucleotide sequence.

Preferably, the melting temperature of the nucleic acid and complementary nucleic acid in physiological fluid (e.g., blood) is greater than body temperature (e.g., the body temperature of a subject, such as a human or mouse). For example, the melting temperature of the nucleic acid and complementary nucleic acid in physiological fluid is preferably greater than 37° C., such as greater than about 38° C., greater than about 39° C., greater than about 40° C., greater than about 41° C., greater than about 42° C., greater than about 43° C., greater than about 44° C., or greater than about 45° C. The melting temperature of the nucleic acid and complementary nucleic acid may be about 37° C. to about 120° C., such as about 38° C. to about 120° C., about 39° C. to about 120° C., about 40° C. to about 120° C., about 41° C. to about 120° C., about 42° C. to about 120° C., about 43° C. to about 120° C., about 44° C. to about 120° C., about 45° C. to about 120° C., about 46° C. to about 120° C., about 47° C. to about 120° C., about 48° C. to about 120° C., about 49° C. to about 120° C., about 50° C. to about 120° C., about 38° C. to about 100° C., about 39° C. to about 100° C., about 40° C. to about 100° C., about 41° C. to about 100° C., about 42° C. to about 100° C., about 43° C. to about 100° C., about 44° C. to about 100° C., about 45° C. to about 100° C., about 46° C. to about 100° C., about 47° C. to about 100° C., about 48° C. to about 100° C., about 49° C. to about 100° C., or about 50° C. to about 100° C.

The length of the nucleic acid of the reactive group, nucleotide sequence of the reactive group, complementary nucleic acid, and complementary nucleotide sequence is preferably greater than 9 nucleotides. The length of the nucleic acid of the reactive group, nucleotide sequence of the reactive group, complementary nucleic acid, and complementary nucleotide sequence may be greater than 10, 11, 12, 13, 14, 15, 16, 17, 18, 19, or 20 nucleotides. The length of the nucleic acid of the reactive group, nucleotide sequence of the reactive group, complementary nucleic acid, and complementary nucleotide sequence may be about 10 nucleotides to about 100 nucleotides, such as about 11 nucleotides to about 80 nucleotides, about 12 nucleotides to about 60 nucleotides, about 13 nucleotides to about 50 nucleotides, about 14 nucleotides to about 40 nucleotides, about 15 nucleotides to about 30 nucleotides, or about 16 nucleotides to about 25 nucleotides. The GC content of the nucleic acid, nucleotide sequence, complementary nucleic acid, and complementary nucleotide sequence may be about 10% to about 100%, such as about 40% to about 100%, about 45% to about 100%, about 50% to about 100%, about 55% to about 100%, about 40% to about 95%, about 45% to about 90%, about 50% to about 85%, or about 55% to about 80%.

In some embodiments, a particle may be cleared by an organism in about 1 day to about 5 years, such as about 1 day to about 3 years, or about 1 day to about 1 year.

### XIII. Methods of Administration

The disclosure contemplates that compositions described herein (e.g., any of the generally or specifically described particles or plurality of particles described herein) may be administered to cells and tissues in vitro and/or in vivo. Administration in vivo includes administration to an animal model of disease, such as an animal model of cancer, or administration to a subject in need thereof. Suitable cells, tissues, or subjects include animals, such as companion animals, livestock, zoo animals, endangered species, rare animals, non-human primates, and humans. Exemplary companion animals include dogs and cats.

For delivery in vitro, such as to and/or around cells or tissues in culture, compositions may be added to the culture media, such as to contact the microenvironment or contact soluble material in the culture media or to contact the cell or even to penetrate the cell. The desired site of activity influences the delivery mechanism and means for administering the compositions (e.g., particles described herein).

For delivery in vivo, such as to cells or tissues in vivo (including to the microenvironment of cells and tissue) and/or to a subject in need thereof, numerous methods of administration are envisioned. The particular method may be selected based on the particle composition and the particular application and the patient. Various delivery systems are known and can be used to administer agents of the disclosure. Any such methods may be used to administer any of the agents described herein. Methods of introduction can be enteral or parenteral, including but not limited to, intradermal, intramuscular, intraperitoneal, intramyocardial, intravenous, subcutaneous, pulmonary, intranasal, intraocular, epidural, and oral routes. A composition of the disclosure may be administered by any convenient route, for example, by infusion or bolus injection, by absorption through epithelial or mucocutaneous linings (e.g., oral mucosa, rectal and intestinal mucosa, etc.) and may be administered together (either concurrently or consecutively) with other biologically active agents. Administration can be systemic or local.

In certain embodiments, a composition is administered intravenously, such as by bolus inject or infusion. In certain embodiments, a composition is administered orally, subcutaneously, intramuscularly or intraperitoneally.

In certain embodiments, it may be desirable to administer a composition of the disclosure locally to the area in need of treatment (e.g., to the site of a tumor, such as by injection into the tumor).

The liver is a frequent site of metastases. Thus, in certain embodiments, delivery of an composition described herein is directed to the liver. For example, a venous catheter may be placed in the hepatic portal vein to deliver agent of the disclosure to the liver. Other methods of delivery via the hepatic portal vein are also contemplated.

In certain embodiments, compositions of the disclosure are administered by intravenous infusion. In certain embodiments, the a composition is infused over a period of at least 10, at least 15, at least 20, or at least 30 minutes. In other embodiments, the agent is infused over a period of at least 60, 90, or 120 minutes. Regardless of the infusion period, the disclosure contemplates that, in certain embodiments, each infusion is part of an overall treatment plan where agent is administered according to a regular schedule (e.g., weekly, monthly, etc.) for some period of time. However, in other embodiments, a composition is delivered by bolus injection, e.g., as part of an overall treatment plan where agent is administered according to a regular schedule for some period of time.

For any of the foregoing, it is contemplated that compositions of the disclosure (include one agent or a combination of two or more such agents) may be administered in vitro or in vivo via any suitable route or method. Compositions may be administered as part of a therapeutic regimen where a composition is administered one time or multiple times, including according to a particular schedule. Moreover, it is contemplated that the compositions of the disclosure will be formulated as appropriate for the route of administration and particular application. The disclosure contemplates any combination of the foregoing features, as well as combinations with any of the aspects and embodiments of the disclosure described herein.

The foregoing applies to any compositions (e.g., a particle or plurality of particles) of the disclosure, used alone or in combination, and used for any of the methods described herein. The disclosure specifically contemplates any combination of the features of such compositions of the disclosure, compositions, and methods with the features described for the various pharmaceutical compositions and routes of administration described in this section and below.

### XIV. Pharmaceutical Compositions

In certain embodiments, the subject particle or particles of the present disclosure are formulated with a pharmaceutically acceptable carrier. One or more compositions (e.g., comprising a particle or plurality of particles described herein) can be administered alone or as a component of a pharmaceutical formulation (composition). Any of the compositions of the disclosure generally or specifically described herein may be formulated, as described herein. In certain embodiments, the composition includes two or more particles of the disclosure or a particle of the disclosure formulated with a second therapeutic agent.

A composition of the disclosure may be formulated for administration in any convenient way for use in human or veterinary medicine. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the subject particle or particles include, for example, those suitable for oral, nasal, topical, parenteral, rectal, and/or intravaginal administration. The formulations may conveniently be presented in unit dosage form and may be prepared by any methods well known in the art of pharmacy. The amount of active ingredient which can be combined with a carrier material to produce a single dosage form will vary depending upon the host being treated and the particular mode of administration. The amount of active ingredient which can be combined with a carrier material to produce a single dosage form will generally be that amount of the compound which produces a therapeutic effect.

In certain embodiments, methods of preparing these formulations or compositions include combining one or more particles and a carrier and, optionally, one or more accessory ingredients. In general, the formulations can be prepared with a liquid carrier, or a finely divided solid carrier, or both, and then, if necessary, shaping the product.

Formulations for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a particle of the disclosure. Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

In solid dosage forms for oral administration (capsules, tablets, pills, dragees, powders, granules, and the like), one or more compositions of the present disclosure may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like. Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

In certain embodiments, methods of the disclosure include topical administration, either to skin or to mucosal membranes such as those on the cervix and vagina. The topical formulations may further include one or more of the wide variety of agents known to be effective as skin or stratum corneum penetration enhancers. Examples of these are 2-pyrrolidone, N-methyl-2-pyrrolidone, dimethylacetamide, dimethylformamide, propylene glycol, methyl or isopropyl alcohol, dimethyl sulfoxide, and azone. Additional agents may further be included to make the formulation cosmetically acceptable. Examples of these are fats, waxes, oils, dyes, fragrances, preservatives, stabilizers, and surface active agents. Keratolytic agents such as those known in the art may also be included. Examples are salicylic acid and sulfur. Dosage forms for the topical or transdermal administration include powders, sprays, ointments, pastes, creams, lotions, gels, solutions, patches, and inhalants. The subject agents of the disclosure may be mixed under sterile conditions with a pharmaceutically acceptable carrier, and with any preservatives, buffers, or propellants which may be required. The ointments, pastes, creams and gels may contain, in addition to a subject agent of the disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to a subject agent of the disclosure, excipients such as lactose, talc, silicic acid, aluminum hydroxide, calcium silicates, and polyamide powder, or mixtures of these substances. Sprays can additionally contain customary propellants, such as chlorofluorohydrocarbons and volatile unsubstituted hydrocarbons, such as butane and propane.

Pharmaceutical compositions suitable for parenteral administration may comprise one or more compositions of the disclosure in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

Injectable depot forms are made by forming microencapsule matrices of one or more particles in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

In a preferred embodiment, the compositions of the present disclosure are formulated in accordance with routine procedures as a pharmaceutical composition adapted for intravenous administration to human beings or animals, such as companion animals. Where necessary, the composition may also include a solubilizing agent and a local anesthetic such as lidocaine to ease pain at the site of the injection. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In another embodiment, the compositions (e.g., particle or particles) described herein are formulated for subcutaneous, intraperitoneal, or intramuscular administration to human beings or animals, such as companion animals.

In certain embodiments, the agents and particles of the present disclosure are formulated for local delivery to a tumor, such as for delivery for intratumoral injection.

In certain embodiments, the composition is intended for local administration to the liver via the hepatic portal vein, and the agents and particles may be formulated accordingly.

In certain embodiments, a particular formulation is suitable for use in the context of deliver via more than one route. Thus, for example, a formulation suitable for intravenous infusion may also be suitable for delivery via the hepatic portal vein. However, in other embodiments, a formulation is suitable for use in the context of one route of delivery, but is not suitable for use in the context of a second route of delivery.

The amount of an agent or particle of the disclosure which will be effective in the treatment of a condition, such as cancer, and/or will be effective in neutralizing soluble TNFR and/or will be effective in decreasing the amount or TNF alpha binding activity of soluble TNFR, particularly soluble TNFR present in a tumor microenvironment and, optionally, in plasma and/or will be effective in inhibiting tumor cell proliferation, growth or survival in vitro or in vivo can be determined by standard clinical or laboratory techniques. In addition, in vitro assays may optionally be employed to help identify optimal dosage ranges. The precise dose to be employed in the formulation will also depend on the route of administration, and the seriousness of the condition, and should be decided according to the judgment of the practitioner and each subject's circumstances. Effective doses for administration to humans or animals may be extrapolated from dose-response curves derived from in vitro or animal model test systems.

In certain embodiments, compositions of the disclosure, including pharmaceutical preparations, are non-pyrogenic. In other words, in certain embodiments, the compositions are substantially pyrogen-free. In one embodiment the formulations of the disclosure are pyrogen-free formulations that are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26(1):223 (2000)). When therapeutic proteins are administered in relatively large dosages and/or over an extended period of time (e.g., such as for the patient's entire life), even small amounts of harmful and dangerous endotoxin could be dangerous. In certain specific embodiments, the endotoxin and pyrogen concentrations in the composition are less than 10 EU/mg, or less than 5 EU/mg, or less than 1 EU/mg, or less than 0.1 EU/mg, or less than 0.01 EU/mg, or less than 0.001 EU/mg.

The foregoing applies to any of the agents of the disclosure, compositions, and methods described herein. The disclosure specifically contemplates any combination of the features of agents of the disclosure described herein, compositions, and methods (alone or in combination) with the features described for the various pharmaceutical compositions and routes of administration described in this section and above.

The disclosure provides numerous general and specific examples of agents and categories of agents suitable for use in the methods of the present disclosure (“agents of the disclosure”). The disclosure contemplates that any such agent or category of agent can be formulated as described herein for administration in vitro or in vivo.

Moreover, in certain embodiments, the disclosure contemplate compositions, including pharmaceutically compositions comprising any agent of the disclosure described herein formulated with one or more pharmaceutically acceptable carrier and/or excipient. Such compositions may be described using any of the functional and/or structural features of an agent of the disclosure provided herein. Any such compositions or pharmaceutical compositions can be used in vitro or in vivo in any of the methods of the disclosure.

Similarly, the disclosure contemplates an isolated or purified agent of the disclosure. An agent of the disclosure described based on any of the functional and/or structural features of an agent described herein may be provided as an isolated agent or a purified agent. Such isolated or purified agents have numerous uses in vitro or in vivo, including use in any of the in vitro or in vivo methods described herein.

### XV. Applications

The compositions (e.g., particles and pharmaceutical compositions thereof) described herein are useful in a variety of diagnostic and therapeutic applications. For example, the particles described herein can be used to treat cancer, detoxify a subject, or treat viral or bacterial infections.

Therapeutic applications include administering one or more of the compositions described herein to a subject, e.g., a human subject, using a variety of methods that depend, in part, on the route of administration. The route can be, e.g., intravenous injection or infusion (IV), subcutaneous injection (SC), intraperitoneal (IP) injection, or intramuscular injection (IM).

Administration can be achieved by, e.g., local infusion, injection, or by means of an implant. The implant can be of a porous, non-porous, or gelatinous material, including membranes, such as sialastic membranes, or fibers. The implant can be configured for sustained or periodic release of the composition to the subject (see, e.g., U.S. Patent Application Publication No. 2008/0241223; U.S. Pat. Nos. 5,501,856; 5,164,188; 4,863,457; and 3,710,795; EP488401; and EP430539, the disclosures of each of which are incorporated by reference in their entirety). The composition can be delivered to the subject by way of an implantable device based on, e.g., diffusive, erodible, or convective systems, e.g., osmotic pumps, biodegradable implants, electrodiffusion systems, electroosmosis systems, vapor pressure pumps, electrolytic pumps, effervescent pumps, piezoelectric pumps, erosion-based systems, or electromechanical systems.

As used herein the term “effective amount” or “therapeutically effective amount,” in an in vivo setting, means a dosage sufficient to treat, inhibit, or alleviate one or more symptoms of the disorder being treated or to otherwise provide a desired pharmacologic and/or physiologic effect, e.g., modulate (e.g., enhance) an immune response to an antigen. The precise dosage will vary according to a variety of factors such as subject-dependent variables (e.g., age, immune system health, etc.), the disease, and the treatment being effected.

In some aspects, the invention relates to a method of treating or preventing a disease or condition in a patient by administering a composition comprising nanoparticles as described herein to the patient. In some embodiments, the invention relates to a method of reducing the concentration of a biomolecule in a patient, such as the concentration of the biomolecule in a bodily fluid of the patient (e.g., blood and/or extracellular fluid), by administering a composition comprising nanoparticles as described herein to the patient.

As used herein, a mammal can be a human, a non-human primate (e.g., monkey, baboon, or chimpanzee), a horse, a cow, a pig, a sheep, a goat, a dog, a cat, a rabbit, a guinea pig, a gerbil, a hamster, a rat, or a mouse. In some embodiments, the mammal is an infant (e.g., a human infant). In certain preferred embodiments, the subject is a human.

As used herein, a subject mammal “in need of prevention,” “in need of treatment,” or “in need thereof,” refers to one, who by the judgment of an appropriate medical practitioner (e.g., a doctor, a nurse, or a nurse practitioner in the case of humans; a veterinarian in the case of non-human mammals), would reasonably benefit from a given treatment.

The term “preventing” is art-recognized, and when used in relation to a condition, is well understood in the art, and includes administration of a composition which reduces the frequency of, or delays the onset of, symptoms of a medical condition in a subject mammal relative to a subject which does not receive the composition.

Suitable human doses of any of the compositions described herein can further be evaluated in, e.g., Phase I dose escalation studies. See, e.g., van Gurp et al., Am J Transplantation 8(8):1711-1718 (2008); Hanouska et al., Clin Cancer Res 13(2, part 1):523-531 (2007); and Hetherington et al., Antimicrobial Agents and Chemotherapy 50(10):3499-3500 (2006).

A method may further comprising measuring the concentration of a biomolecule of interest in a subject (e.g., in the serum of the blood of the subject) prior to administering to the subject a composition comprising a plurality of particles that target the biomolecule. A method may further comprise calculating the number of particles to administer to a subject, e.g., based on the concentration of the biomolecule in the subject (e.g., in the serum of the blood of the subject) and/or the height, weight, and/or age of the subject.

Toxicity and therapeutic efficacy of such compositions can be determined by known pharmaceutical procedures in cell cultures or experimental animals (e.g., animal models of cancer, toxicity, or infection). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Agents that exhibit a high therapeutic index are preferred. While compositions that exhibit toxic side effects may be used, care should be taken to design a delivery system that targets such compounds to the site of affected tissue and to minimize potential damage to normal cells and, thereby, reduce side effects.

The data obtained from the cell culture assays and animal studies can be used in formulating a range of dosage for use in humans. The dosage of such compositions lies generally within a range of circulating concentrations of the compositions that include the ED₅₀ with little or no toxicity. The dosage may vary within this range depending upon the dosage form employed and the route of administration utilized. A therapeutically effective dose can be estimated initially from cell culture assays. A dose can be formulated in animal models to achieve a circulating plasma concentration range that includes the IC₅₀ (i.e., the concentration of the antibody which achieves a half-maximal inhibition of symptoms) as determined in cell culture. Such information can be used to more accurately determine useful doses in humans. Plasma concentrations may be measured, for example, by high performance liquid chromatography (HPLC). In some embodiments, e.g., where local administration is desired, cell culture or animal modeling can be used to determine a dose required to achieve a therapeutically effective concentration within the local site.

In some embodiments of any of the methods described herein, a particle can be administered to a mammal in conjunction with one or more additional therapeutic agents (e.g., therapeutic agents for treating an infection or treating cancer).

In some embodiments, the particle and additional therapeutic agent can be administered to the mammal using different routes of administration. For example, the additional therapeutic agent can be administered subcutaneously or intramuscularly and the particle can be administered intravenously.

In some embodiments, a method of the invention comprises measuring the concentration of a biomolecule in a subject. For example, the method may comprise measuring the concentration of a biomolecule in the blood of a subject. The method may further comprise administering to the subject a composition comprising a plurality of particles that target the biomolecule (i.e., a plurality of particles as described herein, comprising an agent that selectively binds to the biomolecule). The measuring step may allow for the appropriate dosing of the particles. Thus, the measuring step may be performed prior to administering the composition. Nevertheless, the measuring step may be performed after administering the composition, e.g., to assess the efficacy of the composition. The method may further comprise administering to the subject a second or subsequent dose of a composition comprising a plurality of particles, e.g., if warranted in light of the measured concentration of the biomolecule. In this way, the concentration of a biomolecule may be titrated, e.g., by iteratively measuring the concentration of the biomolecule in the subject and administering the composition at varying doses or rates. Similarly, the number of particles administered to the subject may be titrated against the concentration of the biomolecule that is targeted by the particles.

Titrating either the concentration of a biomolecule in a subject or the number of particles administered to the subject may be particularly useful, for example, when the biomolecule contributes to a deleterious local effect (e.g., in a tumor) but has a beneficial systemic effect. Thus, a plurality of particles may be inserted either into or adjacent to a location in a patient to bind the biomolecule in the location, and the systemic concentration of the biomolecule may be monitored to determine whether additional particles may be safely administered to the subject.

Titrating either the concentration of a biomolecule in a subject or the number of particles administered to the subject may also be useful, for example, to maintain a concentration of the biomolecule within a predetermined range. The predetermined range may be a range that is associated with a healthy state, e.g., wherein the subject is overproducing the biomolecule, or the predetermined range may be a therapeutic range. Such titration may be particularly useful in methods of treating diseases caused by the over-secretion of hormones. For example, a particle may comprise an agent that binds to the biomolecule growth hormone, e.g., for use in a method of treating acromegaly or gigantism, and such particles may be titrated to ensure that levels of growth hormone remain in a healthy range. A particle may comprise an agent that binds to the biomolecule thyroxine and/or triiodothyronine, e.g., for use in a method of treating hyperthyroidism, and such particles may be titrated to ensure that levels of thyroxine and/or triiodothyronine remain in a healthy range. A particle may comprise an agent that binds to the biomolecule adrenocorticotropic hormone or cortisol, e.g., for use in a method of treating Cushing's disease, and such particles may be titrated to ensure that levels of adrenocorticotropic hormone and/or cortisol remain in a healthy range. An example of a therapeutic range includes the titration of a blood clotting factor, such as Factor VIII, Factor IX, or Factor XI, to a range that inhibits blood clotting for a period of time. Such a range may be below a normal, healthy concentration, and yet the therapeutic range may be useful, for example, to inhibit thrombosis or ischemia in certain patients.

### XVI. Adoptive Cell Transfer Therapy

A method may comprise administering a composition comprising a plurality of particles as described herein to a subject who has received adoptive cell transfer therapy (ACT). A method may comprise administering a composition comprising a plurality of particles as described herein to a subject who might benefit from adoptive cell transfer therapy. The method may further comprise administering adoptive cell transfer therapy to the subject, e.g., before, after, or concurrently with the administration of the composition comprising a plurality of particles.

Adoptive cell transfer therapy may comprise administering a composition comprising lymphocytes to a subject. The lymphocytes may be T lymphocytes (i.e., T cells), such as tumor-infiltrating lymphocytes (TILs). In preferred embodiments, the lymphocytes are T lymphocytes, such as tumor-infiltrating lymphocytes. The composition comprising lymphocytes may be substantially free from cells that are not lymphocytes, e.g., the composition may be substantially free from cells and cell fragments derived from myeloid progenitor cells (e.g., erythrocytes, mast cells, basophils, neutrophils, eosinophils, monocytes, macrophages, megakaryocytes, platelets). The composition comprising lymphocytes may be substantially free from cells that are not T cells, e.g., the composition may be substantially free from natural killer cells, B cells, and/or plasma cells. The composition comprising lymphocytes may comprise cells wherein the cells consist essentially of T cells. The composition comprising lymphocytes may be substantially free from cells that are not tumor-infiltrating lymphocytes. The composition comprising lymphocytes may comprise tumor-infiltrating lymphocytes. The composition comprising lymphocytes may comprise cells wherein the cells consist essentially of tumor-infiltrating lymphocytes.

The composition comprising lymphocytes may comprise recombinant lymphocytes, e.g., wherein the lymphocytes comprise an exogenous nucleic acid. For example, the lymphocytes may comprise a chimeric antigen receptor (CAR). Similarly, the lymphocytes may comprise a gene knockout, e.g., which reduces the risk of a graft-versus-host immune response or a host-versus-graft immune response (e.g., for a non-autologous transplant, such as an allogeneic transplant). In some embodiments, the composition comprising lymphocytes may comprise recombinant T cells, such as recombinant tumor-infiltrating lymphocytes, e.g., the lymphocytes may be recombinant T cells, such as recombinant tumor-infiltrating lymphocytes.

Adoptive cell transfer therapy may comprise an autologous transplant or a non-autologous transplant, such as an allogeneic transplant.

The subject may have received adoptive cell transfer therapy about 1 year prior to administering the composition to the subject, such as about 6 months, about 5 months, about 4 months, about 3 months, about 2 months, about 1 month, about 4 weeks, about 3 weeks, about 2 weeks, about 14 days, about 13 days, about 12 days, about 11 days, about 10 days, about 9 days, about 8 days, about 7 days, about 6 days, about 5 days, about 4 days, about 3 days, about 2 days, or 1 day prior to administering the composition to the subject. The method may comprise administering a composition comprising a plurality of particles to a subject less than about 1 year after administering a composition comprising lymphocytes to the subject, such as less than about 6 months, about 5 months, about 4 months, about 3 months, about 2 months, about 1 month, about 4 weeks, about 3 weeks, about 2 weeks, about 14 days, about 13 days, about 12 days, about 11 days, about 10 days, about 9 days, about 8 days, about 7 days, about 6 days, about 5 days, about 4 days, about 3 days, about 2 days, or 1 day after administering a composition comprising lymphocytes to the subject. The method may comprise administering a composition comprising a plurality of particles to a subject within about 1 year of administering a composition comprising lymphocytes to the subject, such as within about 6 months, about 5 months, about 4 months, about 3 months, about 2 months, about 1 month, about 4 weeks, about 3 weeks, about 2 weeks, about 14 days, about 13 days, about 12 days, about 11 days, about 10 days, about 9 days, about 8 days, about 7 days, about 6 days, about 5 days, about 4 days, about 3 days, about 2 days, or within about 1 day of administering a composition comprising lymphocytes to the subject.

Adoptive cell transfer therapy may be particularly effective in subjects who have a neoplasm, such as cervical cancer, breast cancer, lymphoma, leukemia, chronic lymphocytic leukemia, follicular lymphoma, large-cell lymphoma, lymphoblastic leukemia, myeloid leukemia, multiple myeloma, bile duct cancer, colorectal cancer, neuroblastoma, lung cancer, sarcoma, synovial sarcoma, or melanoma. Nevertheless, adoptive cell transfer therapy may be useful to treat other diseases, such as serious or life-threatening infections (e.g., HIV).

### XVII. Selected Applications Related to Neoplasms

In some embodiments, the particles described herein can be useful for treating a subject with cancer. Exemplary agents useful in the particle compositions described herein, and/or soluble biomolecules which can be scavenged by such particles, are described herein (e.g., Table 2) and known in the art. For example, particles capable of scavenging sTNFR, MMP2, MMP9, sIL-2R, sIL-1 receptor, and the like are useful for treating a cancer and/or for enhancing an immune response to a cancer by relieving immune dis-inhibition.

The immune dis-inhibition approach to immunotherapy is based, in part, on the concept that many cancer patients are generally immunologically competent overall but their immune systems are locally inhibited in the microenvironments of their tumors. If this inhibition of the immune system is relieved by administering a particle of the disclosure, the patient's own immune system can act on the tumor. Thus, in certain embodiments, particles of the disclosure provide an immunotherapy approach without the need for hyper-stimulating the patient's immune system by adding exogenous, active cytokines intended to bind cell surface receptors to provoke an immune response and/or without otherwise hyper-stimulating the patient's immune system.

Without being bound by theory, because the cancer patients are, generally, immunologically competent, the ability of lymphocytes to recognize tumor antigens is generally unaffected by the tumor. Thus, lymphocytes are drawn to the tumor microenvironment as they would be to any aberrant cell cluster, at which point cytokines and cytotoxic factors, such as Tumor Necrosis Factor (TNF, such as TNF alpha, the main cytotoxic “sword” of the immune system) cleave from lymphocytes into the microenvironment. If the cancer cells were instead virally infected cells, the TNF (such as TNF alpha) would engage a TNF receptor (TNFR) on the surface of the infected cell, resulting in rapid destruction by either apoptosis or oxidative stress depending on whether an R1 or R2 type receptor for TNF is engaged. In other words, in the context of a normal immune response that is not being stimulated by the presence of a tumor and/or tumor antigens, TNF deployed by lymphocytes would be available to bind cell surface TNF receptors (R1 and/or R2 receptors) as part of mounting an immune response. Even in the tumor context, the lymphocytes are deployed to the tumor site.

However, many types of cancer cells behave differently than other aberrant cell types, such as virally infected cells, in that they overproduce TNF receptors (both types) and shed them into a cloud around the tumor. Thus, the microenvironment of cancer cells and/or tumors includes amounts of soluble TNF receptors. Without being bound by theory, the soluble TNF receptor concentration in the tumor microenvironment exceed that found in the microenvironment of healthy cells, such as healthy cells of the same tissue type. Additionally or alternatively, the rate and extent of TNF receptor shedding is greater for cancer cells than from healthy cells. Moreover, without being bound by theory, the concentrations of soluble TNF receptor found in the plasma of cancer patients may, in certain embodiments, be higher than in healthy patients.

Regardless of the mechanism, in this model, these shed, soluble TNF receptors bind to the TNF endogenously released by the recruited lymphocytes, neutralizing the endogenous TNF and effectively creating a bubble of immunologic privilege around the tumor, within which the tumor continues to grow and shed additional TNF receptors. In other words, the shed, soluble TNF receptors soak up the TNF alpha endogenously produced by lymphocytes and prevent or inhibit that TNF from binding cell surface TNF receptors on the cancer cells. This decreases or eliminates the TNF available to bind cell surface TNF receptors on the cancer cells. The soluble TNF receptors essentially outcompete for binding to TNF alpha, and thus, decrease the activity of TNF, such as TNF alpha for binding cell surface TNF receptors.

The above scenario can similarly play out in the context of IL-2 and shed, soluble IL-2 receptors.

In some embodiments, the biomolecule is a toxin released by a cancer cell upon apoptosis.

The present disclosure provides pharmacologic approaches that can be deployed systemically or locally to relieve the inhibition of the immune system created by shed receptors in cancer (e.g., immune dis-inhibition). The present disclosure provides methods and compositions for decreasing the amount and/or activity (e.g., neutralizing the activity) of soluble TNF receptors and/or soluble IL-2 receptors (or any other soluble biomolecules that result in immune dis-inhibition) such as in the microenvironment of cancer cells and tumors. Without being bound by theory, decreasing the amount and/or activity of, for example, soluble TNF receptors (e.g., such as in the tumor microenvironment), may be used as part of a method for inhibiting proliferation, growth, or survival of a cell, such as a cancer cell. In certain embodiments, it may be used for inhibiting survival of a cell, such as a cancer cell. Exemplary methods and agents are described herein.

Regulatory T-cells (TREGs) can secrete the same ligands as cancer cells as a way of tamping down the immune response to avoid, e.g., autoimmune disease caused by overactive T-cells or prolonged T-cell function. For instance, CD80/B7-1 and CD86/B7-2 bind to the CTLA-4 receptor on T-cells and inhibit T-cell activity. Rather than blockading the CTLA-4 receptor, the particles described herein can be designed to scavenge CD80/B7-1 and/or CD86/B7-2. Likewise, the particles described herein can be designed to scavenge other immune checkpoint inhibitors, such as PD-L1, e.g., using particles comprising PD-1 receptor. Such particle compositions offer several benefits over other approaches to stimulating the immune system for the treatment of cancer.

The target may be soluble PD-L2, e.g., to inhibit an interaction between soluble PD-L2 and PD1. The agent may be PD1. Inhibition of an interaction between soluble PD-L2 and PD1 may allow for PD1 to bind a membrane-bound version of PD-L2, thereby favoring apoptosis of a cancer cell. The target may be soluble PD1. The agent may be a ligand of PD1, such as PD-L2, soluble PD-L2 or a variant thereof, or an anti-PD1 antibody, such as nivolumab or pembrolizumab. Particles targeting PD1 (i.e., soluble PD1) and ligands thereof may be particularly useful for treating autoimmune disease, in addition to other diseases and conditions.

The target may be soluble CTLA4, e.g., to inhibit an interaction between B7-1 or B7-2 and soluble CTLA4. The agent may be a ligand of CTLA4 such as soluble B7-1, soluble B7-2 or variants thereof, or an anti-CTLA4 antibody, such as ipilimumab or tremelimumab. Inhibition of interaction between B7-1 or B7-2 and soluble CTLA4 may allow for B7-1 or B7-2 to bind to CD28 on T cells, thereby favoring activation of T cells. Particles targeting CTLA4 (i.e., soluble CTLA4) may be particularly useful for treating melanomas and lung cancer, such as non-small cell lung cancer, in addition to other diseases and conditions.

The agent may be a protein that specifically binds adenosine, such as the adenosine-binding portion of an adenosine receptor. The target may be adenosine. Particles targeting adenosine may be particularly useful for treating solid tumors, and such particles may be injected into a solid tumor, e.g., to inhibit adenosine signaling within the tumor microenvironment.

The agent may be osteoprotegerin or a ligand-binding portion thereof, e.g., for selectively binding ligands of osteoprotegerin. Particles targeting ligands of osteoprotegerin may be particularly useful for treating cancer, such as breast cancer, in addition to other diseases and conditions.

In some embodiments, the subject is one who has, is suspected of having, or is at risk for developing a cancer. In some embodiments, the subject is one who has, is suspected of having, or is at risk for developing an autoimmune disease.

As used herein, a subject “at risk for developing” a cancer is a subject having one or more (e.g., two, three, four, five, six, seven, or eight or more) risk factors for developing a cancer. For example, a subject at risk of developing a cancer may have a predisposition to develop a cancer (i.e., a genetic predisposition to develop a cancer such as a mutation in a tumor suppressor gene (e.g., mutation in BRCA1, p53, RB, or APC) or has been exposed to conditions that can result in the condition). Thus, a subject can be one “at risk of developing a cancer when the subject has been exposed to mutagenic or carcinogenic concentrations of certain compounds (e.g., carcinogenic compounds in cigarette smoke such as acrolein, arsenic, benzene, benz[a]anthracene, benzo[a]pyrene, polonium-210 (Radon), urethane, or vinyl chloride). Moreover, the subject can be “at risk of developing a cancer” when the subject has been exposed to, e large doses of ultraviolet light or X-irradiation, or exposed (e.g., infected) to a tumor-causing/associated virus such as papillomavirus, Epstein-Barr virus, hepatitis B virus, or human T-cell leukemia-lymphoma virus. Cancer is a class of diseases or disorders characterized by uncontrolled division of cells and the ability of these to spread, either by direct growth into adjacent tissue through invasion, or by implantation into distant sites by metastasis (where cancer cells are transported through the bloodstream or lymphatic system). Cancer can affect people at all ages, but risk tends to increase with age. Types of cancers can include, e.g., lung cancer, breast cancer, colon cancer, pancreatic cancer, renal cancer, stomach cancer, liver cancer, bone cancer, hematological cancer, neural tissue cancer (e.g., glioblastoma such as glioblastoma multiforme), melanoma, thyroid cancer, ovarian cancer, testicular cancer, prostate cancer, cervical cancer, vaginal cancer, or bladder cancer. In certain preferred embodiments, a patient (or subject) has brain cancer, endometrial cancer, prostate cancer, renal cancer, or squamous cell cancer (e.g., squamous cell cancer of the head and neck), each of which are particularly sensitive to extracellular biomolecules that may exacerbate the disease.

Similarly, a subject at risk for developing an infection is one having one or more risk factors that increase the likelihood of exposure to a pathogenic microorganism.

A subject “suspected of having” a cancer or an infection is one having one or more symptoms of the cancer or infection. It should be understood that subjects at risk for developing, or suspected of having, a cancer or an infection does not include all subjects within the species of interest.

In some embodiments, the methods include determining whether the subject has a cancer.

### XVIII. Selected Applications Related to Inflammatory and Autoimmune Disorders

In some embodiments, the particles described herein can be used for treating an inflammatory disorder and/or an autoimmune disorder. Exemplary agents useful in the particle compositions described herein, and/or soluble biomolecules which can be scavenged by such particles, are described herein (e.g., Table 2) and known in the art. For example, particles capable of scavenging cytokines (e.g., TNFα or interleukins, such as IL-2, IL-6, or IL-1) or chemokines (e.g., CXCL8 or CXCL1) can be useful for treating a variety of autoimmune and/or inflammatory disorders.

The agent may be soluble CD28 or a ligand-binding portion thereof, e.g., for selectively binding ligands of CD28, such as soluble B7 (e.g., soluble B7-1 or soluble B7-2). The agent may be galiximab. The target may be a ligand of CD28, such as soluble B7. Particles targeting ligands of CD28 may be particularly useful for preventing or treating lupus, such as systemic lupus erythematosus, in addition to other diseases and conditions.

The agent may be an anti-B7-H4 antibody, e.g., for selectively binding soluble B7-H4. The target may be soluble B7-H4. Particles targeting soluble B7-H4 may be particularly useful for treating arthritis, such as rheumatoid arthritis and juvenile idiopathic arthritis, in addition to other diseases and conditions.

The agent may be soluble CD278 (inducible co-stimulator; “ICOS”) or a ligand-binding portion thereof, e.g., for selectively binding ligands of CD278, such as ICOSL (inducible co-stimulator ligand; CD275). The target may be a ligand of CD278, such as ICOSL. Particles targeting ligands of CD278 may be particularly useful for preventing or treating lupus, such as systemic lupus erythematosus, in addition to other diseases and conditions.

The agent may be an anti-CD275 antibody, e.g., for selectively binding CD275 (inducible co-stimulator ligand; “ICOSL”). The target may be CD275. Particles targeting CD275 may be particularly useful for preventing or treating lupus, such as systemic lupus erythematosus, in addition to other diseases and conditions.

The agent may be an anti-CD40L antibody, such as dapirolizumab, ruplizumab, or toralizumab, e.g., for selectively binding CD40L (CD40 Ligand; CD154). The target may be CD40L. Particles targeting CD40L may be particularly useful for preventing or treating lupus, such as systemic lupus erythematosus, arthritis, such as rheumatoid arthritis, collagen-induced arthritis, and juvenile idiopathic arthritis, and Sjogren's syndrome, in addition to other diseases and conditions.

The agent may be soluble CD134 (OX40) or a ligand-binding portion thereof, e.g., for selectively binding ligands of CD134, such as CD252 (OX40 ligand; “OX40L”). The target may be a ligand of CD134, such as CD252. Particles targeting ligands of CD134 may be particularly useful for preventing or treating lupus, such as lupus nephritis, symptoms thereof, such as glomerulonephritis, and systemic sclerosis, in addition to other diseases and conditions.

The agent may be 4-1BB (CD137) or a ligand-binding portion thereof, e.g., for selectively binding ligands of 4-1BB, such as soluble 4-1BB ligand (soluble 4-1BBL). The target may be a ligand of 4-1BB, such as soluble 4-1BB ligand. Particles targeting ligands of 4-1BB may be particularly useful for preventing or treating lupus, such as systemic lupus erythematosus, and arthritis, such as rheumatoid arthritis, in addition to other diseases and conditions.

The agent may be 4-1BB ligand, e.g., for selectively binding soluble 4-1BB (soluble CD137). The agent may be an anti-4-1BB antibody, such as urelumab. The target may be soluble 4-1BB. Particles targeting soluble 4-1BB may be particularly useful for preventing or treating arthritis, such as rheumatoid arthritis, in addition to other diseases and conditions, including cancer. In some embodiments, the inflammatory disorder can be, e.g., acute disseminated encephalomyelitis; Addison's disease; Ankylosing spondylitis; Antiphospholipid antibody syndrome; Autoimmune hemolytic anemia; Autoimmune hepatitis; Autoimmune inner ear disease; Bullous pemphigoid; Chagas disease; Chronic obstructive pulmonary disease; Coeliac disease; Dermatomyositis; Diabetes mellitus type 1; Diabetes mellitus type 2; Endometriosis; Goodpasture's syndrome; Graves' disease; Guillain-Barre syndrome; Hashimoto's disease; Idiopathic thrombocytopenic purpura; Interstitial cystitis; Systemic lupus erythematosus (SLE); Metabolic syndrome, Multiple sclerosis; Myasthenia gravis; Myocarditis, Narcolepsy; Obesity; Pemphigus Vulgaris; Pernicious anaemia; Polymyositis; Primary biliary cirrhosis; Rheumatoid arthritis; Schizophrenia; Scleroderma; Sjogren's syndrome; Vasculitis; Vitiligo; Wegener's granulomatosis; Allergic rhinitis; Prostate cancer; Non-small cell lung carcinoma; Ovarian cancer; Breast cancer; Melanoma; Gastric cancer; Colorectal cancer; Brain cancer; Metastatic bone disorder; Pancreatic cancer; a Lymphoma; Nasal polyps; Gastrointestinal cancer; Ulcerative colitis; Crohn's disorder; Collagenous colitis; Lymphocytic colitis; Ischaemic colitis; Diversion colitis; Behcet's syndrome; Infective colitis; Indeterminate colitis; Inflammatory liver disorder, Endotoxin shock, Rheumatoid spondylitis, Ankylosing spondylitis, Gouty arthritis, Polymyalgia rheumatica, Alzheimer's disorder, Parkinson's disorder, Epilepsy, AIDS dementia, Asthma, Adult respiratory distress syndrome, Bronchitis, Cystic fibrosis, Acute leukocyte-mediated lung injury, Distal proctitis, Wegener's granulomatosis, Fibromyalgia, Bronchitis, Cystic fibrosis, Uveitis, Conjunctivitis, Psoriasis, Eczema, Dermatitis, Smooth muscle proliferation disorders, Meningitis, Shingles, Encephalitis, Nephritis, Tuberculosis, Retinitis, Atopic dermatitis, Pancreatitis, Periodontal gingivitis, Coagulative Necrosis, Liquefactive Necrosis, Fibrinoid Necrosis, Hyperacute transplant rejection, Acute transplant rejection, Chronic transplant rejection, Acute graft-versus-host disease, Chronic graft-versus-host disease, or combinations of any of the foregoing. In some embodiments, the autoimmune or inflammatory disorder can be, e.g., colitis, multiple sclerosis, arthritis, rheumatoid arthritis, osteoarthritis, juvenile arthritis, psoriatic arthritis, acute pancreatitis, chronic pancreatitis, diabetes, insulin-dependent diabetes mellitus (IDDM or type I diabetes), insulitis, inflammatory bowel disease, Crohn's disease, ulcerative colitis, autoimmune hemolytic syndromes, autoimmune hepatitis, autoimmune neuropathy, autoimmune ovarian failure, autoimmune orchitis, autoimmune thrombocytopenia, reactive arthritis, ankylosing spondylitis, silicone implant associated autoimmune disease, Sjogren's syndrome, systemic lupus erythematosus (SLE), vasculitis syndromes (e.g., giant cell arteritis, Behcet's disease, and Wegener's granulomatosis), vitiligo, secondary hematologic manifestation of autoimmune diseases (e.g., anemias), drug-induced autoimmunity, Hashimoto's thyroiditis, hypophysitis, idiopathic thrombocytic pupura, metal-induced autoimmunity, myasthenia gravis, pemphigus, autoimmune deafness (e.g., Meniere's disease), Goodpasture's syndrome, Graves' disease, HIV-related autoimmune syndromes, and/or Gullain-Barre disease.

In some embodiments, the autoimmune or inflammatory disorder is a hypersensitivity reaction. As used herein, “hypersensitivity” refers to an undesirable immune system response. Hypersensitivity is divided into four categories. Type I hypersensitivity includes allergies (e.g., Atopy, Anaphylaxis, or Asthma). Type II hypersensitivity is cytotoxic/antibody mediated (e.g., Autoimmune hemolytic anemia, Thrombocytopenia, Erythroblastosis fetalis, or Goodpasture's syndrome). Type III is immune complex diseases (e.g., Serum sickness, Arthus reaction, or SLE). Type IV is delayed-type hypersensitivity (DTH), Cell-mediated immune memory response, and antibody-independent (e.g., Contact dermatitis, Tuberculin skin test, or Chronic transplant rejection). As used herein, “allergy” means a disorder characterized by excessive activation of mast cells and basophils by IgE. In certain instances, the excessive activation of mast cells and basophils by IgE results (either partially or fully) in an inflammatory response. In certain instances, the inflammatory response is local. In certain instances, the inflammatory response results in the narrowing of airways (i.e., bronchoconstriction). In certain instances, the inflammatory response results in inflammation of the nose (i.e., rhinitis). In certain instances, the inflammatory response is systemic (i.e., anaphylaxis).

In some embodiments, the methods include determining whether the subject has an autoimmune disease.

### XIX. Selected Applications Related to Pathogens and Toxins

In some embodiments, the particles described herein can be designed to bind to microorganisms (e.g., viruses or bacteria) or components of microorganisms, such as endotoxin. Accordingly, the particles described herein can be useful to treat, e.g., an infectious disease (e.g., viral infectious diseases including HPV, HBV, hepatitis C Virus (HCV), retroviruses such as human immunodeficiency virus (HIV-1 and HIV-2), herpes viruses such as Epstein Barr Virus (EBV), cytomegalovirus (CMV), HSV-1 and HSV-2, and influenza virus. In addition, bacterial, fungal and other pathogenic infections are included, such as Aspergillus, Brugia, Candida, Chlamydia, Coccidia, Cryptococcus, Dirofilaria, Gonococcus, Histoplasma, Leishmania, Mycobacterium, Mycoplasma, Paramecium, Pertussis, Plasmodium, Pneumococcus, Pneumocystis, Rickettsia, Salmonella, Shigella, Staphylococcus, Streptococcus, Toxoplasma and Vibriocholerae. Exemplary species include Neisseria gonorrhea, Mycobacterium tuberculosis, Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus, Brucella melitensis, Brucella suis, Brucella canis, Campylobacter fetus, Campylobacter fetus intestinalis, Leptospira pomona, Listeria monocytogenes, Brucella ovis, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Babesia caballi, Clostridium tetani, Clostridium botulinum; or, a fungus, such as, e.g., Paracoccidioides brasiliensis; or other pathogen, e.g., Plasmodium falciparum. Also included are National Institute of Allergy and Infectious Diseases (NIAID) priority pathogens. These include Category A agents, such as variola major (smallpox), Bacillus anthracis (anthrax), Yersinia pestis (plague), Clostridium botulinum toxin (botulism), Francisella tularensis (tularaemia), filoviruses (Ebola hemorrhagic fever, Marburg hemorrhagic fever), arenaviruses (Lassa (Lassa fever), Junin (Argentine hemorrhagic fever), and related viruses); Category B agents, such as Coxiella burnetti (Q fever), Brucella species (brucellosis), Burkholderia mallei (glanders), alphaviruses (Venezuelan encephalomyelitis, eastern & western equine encephalomyelitis), ricin toxin from Ricinus communis (castor beans), epsilon toxin of Clostridium perfringens; Staphylococcus enterotoxin B, Salmonella species, Shigella dysenteriae, Escherichia coli strain 0157:H7, Vibrio cholerae, Cryptosporidium parvum; Category C agents, such as nipah virus, hantaviruses, tickborne hemorrhagic fever viruses, tickborne encephalitis viruses, yellow fever, and multidrug-resistant tuberculosis; helminths, such as Schistosoma and Taenia; and protozoa, such as Leishmania (e.g., L. mexicana), and Plasmodium.

The target may be a viral protein. The viral protein may be from arbovirus, adenovirus, alphavirus, arenaviruses, astrovirus, BK virus, bunyaviruses, calicivirus, cercopithecine herpes virus 1, Colorado tick fever virus, coronavirus, Coxsackie virus, Crimean-Congo hemorrhagic fever virus, cytomegalovirus, Dengue virus, ebola virus, echinovirus, echovirus, enterovirus, Epstein-Barr virus, flavivirus, foot-and-mouth disease virus, hantavirus, hepatitis A, hepatitis B, hepatitis C, herpes simplex virus I, herpes simplex virus II, human herpes virus, human immunodeficiency virus type I (HIV-I), human immunodeficiency virus type II (HIV-II), human papillomavirus, human T-cell leukemia virus type I, human T-cell leukemia virus type II, influenza, Japanese encephalitis, JC virus, Junin virus, lentivirus, Machupo virus, Marburg virus, measles virus, mumps virus, naples virus, norovirus, Norwalk virus, orbiviruses, orthomyxovirus, papillomavirus, papovavirus, parainfluenza virus, paramyxovirus, parvovirus, picornaviridae, poliovirus, polyomavirus, poxvirus, rabies virus, reovirus, respiratory syncytial virus, rhinovirus, rotavirus, rubella virus, sapovirus, smallpox, togaviruses, Toscana virus, varicella zoster virus, West Nile virus, or Yellow Fever virus. The viral protein may be, for example, a viral capsid protein or a viral envelope protein.

The target may be a bacterial protein or a component of a bacterial cell wall. For example, the bacterial protein or cell wall component may be from Actinomyces israelii, Bacillus anthracis, Bacillus cereus, Bacteroides fragilis, Bartonella henselae, Bartonella Quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diptheriae, Ehrlichia canis, Ehrlichia chaffeensis, Enterococcus faecalis, Enterococcus faecium, Escherichia coli, Francisella tularensis, Haemophilus influenzae, Haemophilus vaginalis, Helicobacter pylori, Klebsiella pneumoniae, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Nocardia asteroides, Rickettsia rickettsii, Salmonella typhi, Salmonella typhimurium, Shigella sonnei, Shigella dysenteriae, Staphylococcus aureus, Staphylococcus epidermidis, Staphylococcus saprophyticus, Streptococcus agalactiae, Streptococcus pneumoniae, Streptococcus pyogenes, Streptococcus viridans, Treponema pallidum, Ureaplasma urealyticum, Vibrio cholerae, Yersinia pestis, Yersinia enterocolitica, or Yersinia pseudotuberculosis.

The target may be a yeast or fungal protein or a component of a yeast or fungal cell wall. For example, the yeast or fungal protein or cell wall component may be from Apophysomyces variabilis, Aspergillus clavatus, Aspergillus flavus, Aspergillus fumigatus, Basidiobolus ranarum, Candida albicans, Candida glabrata, Candida guilliermondii, Candida krusei, Candida lusitaniae, Candida parapsilosis, Candida tropicalis, Candida stellatoidea, Candida viswanathii, Conidiobolus coronatus, Conidiobolus incongruous, Cryptococcus albidus, Cryptococcus gattii, Cryptococcus laurentii, Cryptococcus neoformans, Encephalitozoon intestinalis, Enterocytozoon bieneusi, Exophiala jeanselmei, Fonsecaea compacta, Fonsecaea pedrosoi, Geotrichum candidum, Histoplasma capsulatum, Lichtheimia corymbifera, Mucor indicus, Paracoccidioides brasiliensis, Phialophora verrucosa, Pneumocystis carinii, Pneumocystis jirovecii, Pseudallescheria boydii, Rhinosporidium seeberi, Rhodotorula mucilaginosa, Stachybotrys chartarum, Syncephalastrum racemosum, or Rhizopus oryzae.

The target may be a protozoan protein. The protozoan protein may be from Cryptosporidium, Giardia intestinalis, Giardia lamblia, Leishmania aethiopica, Leishmania braziliensis, Leishmania donovani, Leishmania infantum, Leishmania major, Leishmania mexicana, Leishmania tropica, Plasmodium coatneyi, Plasmodium falciparum, Plasmodium garnhami, Plasmodium inui, Plasmodium odocoilei, Trichomonas gallinae, Trichomonas vaginalis, Tritrichomonas foetus, Trypanosoma brucei, Trypanosoma cruzi, Trypanosoma equiperdum, Trypanosoma evansi, Trypanosoma lewisi, Trypanosoma pestanai, Trypanosoma suis, or Trypanosoma vivax,

The target may be a toxin, such as a bacterial toxin, a plant toxin, or a zootoxin. The toxin may be, for example, melittin, brevetoxin, tetrodotoxin, chlorotoxin, tetanus toxin, bungarotoxin, Clostridium botulinum toxin, ricin, epsilon toxin of Clostridium perfringens, Staphylococcus enterotoxin B, or endotoxin.

The target may be a bacterial cell-surface lipopolysaccharide, lipopolysaccharide-binding protein, lipoteichoic acid, a bacterial lipoprotein, a bacterial peptidoglycan, lipoarabinomannan, a bacterial flagella protein (e.g., flagellin), profilin, HSP70, zymosan, double-stranded RNA, bacterial ribosomal RNA, or DNA comprising unmethylated CpG.

In some aspects, the invention relates to a method of treating or preventing an infection caused by a pathogen, comprising administering to a subject a composition comprising a plurality of particles as described herein. In some embodiments, the particle comprises an agent that specifically binds to a biomolecule of a pathogen, or a biomolecule produced by the pathogen. In some embodiments, the particle comprises an agent that specifically binds to a biomolecule of the subject (e.g., a biomolecule produced by the subject), such as a cytokine or peroxiredoxin (e.g., peroxiredoxin 1 or peroxiredoxin 2). For example, a method may comprise administering to a subject a composition comprising a plurality of particles that selectively bind TNFα, interleukin 1, interleukin 6, interleukin 8, interleukin 12, interferon gamma, macrophage migration inhibitory factor, GM-C SF, and/or a blood clotting factor, e.g., to treat or prevent sepsis associated with an infection caused by a pathogen. In some embodiments, the method is a method of treating or preventing sepsis, e.g., comprising administering to a subject a composition comprising a plurality of particles as described herein.

The target may be paracetamol (acetaminophen). The agent may be an antibody that specifically binds paracetamol, or an antigen-binding portion thereof. Particles that target paracetamol may be particularly useful for treating or preventing paracetamol toxicity.

### XX. Selected Applications Related to Diet and Metabolism

In some embodiments, the particles described herein can be used to treat obesity, an eating disorder, reduce body mass, promote healthy eating, or reduce the appetite of a subject. For example, in some embodiments, particles comprising agents (e.g., antibodies or soluble forms of the ghrelin receptor (GHSR)) that bind to ghrelin can be administered to a subject (e.g., an overweight or obese subject) to reduce the subject's appetite, treat obesity or an obesity-related disorder, or a metabolic disorder.

As used herein, a metabolic disorder can be any disorder associated with metabolism, and examples include but are not limited to, obesity, central obesity, insulin resistance, glucose intolerance, abnormal glycogen metabolism, type II diabetes, hyperlipidemia, hypoalbuminemia, hypertriglyceridemia, metabolic syndrome, syndrome X, a fatty liver, fatty liver disease, polycystic ovarian syndrome, and acanthosis nigricans.

“Obesity” refers to a condition in which the body weight of a mammal exceeds medically recommended limits by at least about 20%, based upon age and skeletal size. “Obesity” is characterized by fat cell hypertrophy and hyperplasia. “Obesity” may be characterized by the presence of one or more obesity-related phenotypes, including, for example, increased body mass (as measured, for example, by body mass index, or “BMI”), altered anthropometry, basal metabolic rates, or total energy expenditure, chronic disruption of the energy balance, increased Fat Mass as determined, for example, by DEXA (Dexa Fat Mass percent), altered maximum oxygen use (VO₂), high fat oxidation, high relative resting rate, glucose resistance, hyperlipidemia, insulin resistance, and hyperglycemia. See also, for example, Hopkinson et al., Am J Clin Nutr 65(2):432-8 (1997) and Butte et al., Am J Clin Nutr 69(2):299-307 (1999). “Overweight” individuals are generally having a body mass index (BMI) between 25 and 30. “Obese” individuals or individuals suffering from “obesity” are generally individuals having a BMI of 30 or greater. Obesity may or may not be associated with insulin resistance.

An “obesity-related disease” or “obesity related disorder” or “obesity related condition,” which are all used interchangeably, refers to a disease, disorder, or condition, which is associated with, related to, and/or directly or indirectly caused by obesity. The “obesity-related diseases,” or the “obesity-related disorders” or the “obesity related conditions” include but are not limited to, coronary artery disease/cardiovascular disease, hypertension, cerebrovascular disease, stroke, peripheral vascular disease, insulin resistance, glucose intolerance, diabetes mellitus, hyperglycemia, hyperlipidemia, dyslipidemia, hypercholesteremia, hypertriglyceridemia, hyperinsulinemia, atherosclerosis, cellular proliferation and endothelial dysfunction, diabetic dyslipidemia, HIV-related lipodystrophy, peripheral vessel disease, cholesterol gallstones, cancer, menstrual abnormalities, infertility, polycystic ovaries, osteoarthritis, sleep apnea, metabolic syndrome (Syndrome X), type II diabetes, diabetic complications including diabetic neuropathy, nephropathy, retinopathy, cataracts, heart failure, inflammation, thrombosis, congestive heart failure, and any other cardiovascular disease related to obesity or an overweight condition and/or obesity related asthma, airway, and pulmonary disorders.

In yet another aspect, the disclosure features a method for increasing muscle mass or muscle strength in a subject in need thereof, which method comprises administering to the subject one or more of the compositions described herein in an amount sufficient to increase muscle mass or muscle strength in the subject. For example, particles comprising an agent (e.g., an antibody or soluble activin receptor) that binds to myostatin can be administered to a subject to increase muscle mass.

In some embodiments, the subject is one having a muscle disorder (e.g., a muscle wasting disorder).

A muscle wasting disorder, as used herein, encompasses disorders or conditions in which muscle wasting is one of the primary symptoms, such as muscular dystrophy, spinal cord injury, neurodegenerative diseases, anorexia, sarcopenia, cachexia, muscular atrophy due to immobilization, prolonged bed rest, or weightlessness, and the like, as well as disorders in which an abnormally high fat-to-muscle ratio is implicated in a disease or pre-disease state, e.g., Type II diabetes or Syndrome X.

Atrophy of skeletal muscle occurs in muscles of adult animals as a result of lack of use, aging, starvation, and as a consequence of a variety of diseases, disorders, and conditions such as sepsis, muscular dystrophy, AIDS, aging, and cancer. The loss of muscle is generally characterized by decreases in protein content, force production, fatigue resistance, and muscle fiber diameter. These decreases can be attributed to both a decrease in protein synthesis and an increase in protein degradation. Muscle wasting and related conditions to which the compositions and methods of the invention are directed include any condition in which enhanced muscle growth, or diminishment of muscle wasting, produces a therapeutically or otherwise desirable result. Conditions include muscular dystrophy, sarcopenia, cachexia, diabetes mellitus, and the improvement of muscle mass where such improvement is ethical and desirable, e.g., in food animals.

One class of muscle wasting disorders, as mentioned above, is the muscular dystrophies. These are a heterogeneous group of neuromuscular disorders, which include the most common type, Duchenne muscular dystrophy (DMD), multiple types of limb girdle MD (LGMD) and other congenital MDs (CMD). Progressive muscle damage and muscle loss, tissue inflammation and replacement of healthy muscle with fibrous and fatty tissues result in muscle wasting in muscular dystrophy. Extreme muscle loss is one of the most prominent signs of the disease, and leads to complications and symptoms, including death.

Sarcopenia is the age-related loss of muscle mass, strength and function. It begins in the fourth decade of life and accelerates after the age of approximately 75 years. Many factors, including physical inactivity, motor-unit remodeling, decreased hormone levels, and decreased protein synthesis, may all contribute to sarcopenia. With the exception of physical inactivity, all of these may be subject to genetic control where gene modulation may be useful. For example, the rate of muscle protein synthesis and protein breakdown affects sarcopenia. The balance of protein synthesis and breakdown determines the protein content in the body. Research has consistently reported that muscle protein synthesis rates are lower in older adults when compared to younger adults. A decrease in muscle protein catabolism, effected by, e.g., gene modulation, could result in slowing or reversal of the loss of muscle mass.

### XXI. Selected Applications Related to Aging and Neurodegenerative Disorders

In some embodiments, compositions described herein are useful for promoting healthy aging in subject. For example, particles comprising an agent (e.g., an antibody or soluble form of a receptor) capable of binding to any one of TGFβ1, CCL11, MCP-1/CCL2, beta-2 microglobulin, GDF-8/myostatin, or haptoglobin can be used to promote healthy aging in a subject, extend the lifespan of a subject, prevent or delay the onset of an age-related disorder in a subject, or treat a subject suffering from an age-related disorder. In some embodiments, particles comprising an agent that binds to TGFβ1 can be used to enhance/promote neurogenesis and/or muscle regeneration in a subject, e.g., an elderly subject. In some embodiments, the age-related disorder is a cardiovascular disease. In some embodiments, the age-related disorder is a bone loss disorder. In some embodiments, the age-related disorder is a neuromuscular disorder. In some embodiments, the age-related disorder is a neurodegenerative disorder or a cognitive disorder. In some embodiments, the age-related disorder is a metabolic disorder. In some embodiments, the age-related disorder is sarcopenia, osteoarthritis, chronic fatigue syndrome, Alzheimer's disease, senile dementia, mild cognitive impairment due to aging, schizophrenia, Parkinson's disease, Huntington's disease, Pick's disease, Creutzfeldt-Jakob disease, stroke, CNS cerebral senility, age-related cognitive decline, pre-diabetes, diabetes, obesity, osteoporosis, coronary artery disease, cerebrovascular disease, heart attack, stroke, peripheral arterial disease, aortic valve disease, stroke, Lewy body disease, amyotrophic lateral sclerosis (ALS), mild cognitive impairment, pre-dementia, dementia, progressive subcortical gliosis, progressive supranuclear palsy, thalamic degeneration syndrome, hereditary aphasia, myoclonus epilepsy, macular degeneration, or cataracts.

The biomolecule may be alpha-synuclein, tau, amyloid precursor protein, or amyloid β. For example, a method may comprise administering a composition comprising a plurality of particles to a subject with Alzheimer's disease, and the particles may comprise an agent that specifically binds amyloid β (e.g., soluble amyloid β and/or amyloid β aggregates). The biomolecule may be Aβ40 or Aβ42. The agent may comprise aducanumab, bapineuzumab, crenezumab, gantenerumab, ponezumab, solanezumab, or an antigen-binding portion of any one of the foregoing. Similarly, a method may comprise administering a composition comprising a plurality of particles to a subject with Alzheimer's disease, and the particles may comprise an agent that specifically binds tau.

The biomolecule may be TDP-43 or FUS. The biomolecule may be a prion. The biomolecule may be PrP^{Sc}, a soluble PrP protein, or a PrP aggregate.

### XXII. Selected Diagnostic Applications

The particles described herein are also useful as diagnostic agents, or in conjunction with diagnostic tool or apparatus. For example, the particles described herein can be coupled to a detection device that monitors the concentration of a given soluble ligand of interest. For example, a nano channel in a detection device lined with an agent (e.g., a first member of a binding pair) can detect (e.g., in a blood sample) or monitor (e.g., as an implanted device in a subject) the concentration of a soluble biomolecule (e.g., the second member of a binding pair). Such a detector can be useful, e.g., for determining the effectiveness of the particles described herein (at scavenging the soluble biomolecule) or determine/adjust the appropriate dosage of a particle composition (e.g., increasing a dose or dose frequency to more effectively scavenge a soluble biomolecule).

In some embodiments, the particles described herein and the detection devices are integrated and function as a “microgland” or “nanogland” (see, e.g., Sabek et al., Lab Chip 13(18):3675-3688 (2013)). The nanogland features, e.g., a nano-channel diagnostic capable of providing a precise, quantitative measure of the concentration of a soluble biomolecule in a biological fluid of the subject in which the nanogland is implanted. Also featured in the nanogland is a means (e.g., nano-syringe) that would release particles capable of scavenging the biomolecule, e.g., when the concentration of the biomolecule in the biological fluid reaches a set threshold concentration. Given that many thousands of nano-channels can be deployed in a fingernail-sized implantable biochip, microglands or nanoglands can be designed to monitor many different soluble biomolecules and release multiple types of therapeutic particles.

### XXIII. Selected In Vitro Applications

In some aspects, the invention relates to a method for removing a biomolecule from a composition, comprising contacting the composition with a particle as described herein. Such methods are particularly useful for scientific research. For example, it is relatively easy to add a biomolecule to a solution, however it is somewhat more challenging to remove a specific biomolecule from a solution.

Current techniques for removing a biomolecule from solution include, for example, binding the biomolecule to a particle, such as a sepharose bead, and then physically separating the bead from the solution. The particles described herein may sequester a biomolecule in a composition, thereby inhibiting interactions with other components of the composition (e.g., cells), without the need to physically separate the particles from the composition.

A particle may comprise a fluorophore. A particle may be magnetic or paramagnetic or a particle may comprise a magnetic or paramagnetic subparticle or component that allows the particle to be attracted to a magnetic field.

A method may comprise contacting a composition with a particle as described herein, wherein the composition is a cell culture. For example, the cell culture may be a bacterial cell culture or a tissue culture. Such methods may be useful, for example, to remove a secreted protein from the cell culture or to remove a contaminant from the cell culture.

A method may comprise contacting a composition with a particle as described herein, wherein the composition is a cell lysate. The cell lysate may be a prokaryotic or eukaryotic cell lysate. Such methods may be useful, for example, to inhibit the activity of a target biomolecule.

The above methods may be particularly useful for assessing the function of a biomolecule of interest in a particular system. For example, the biomolecule may be introduced to a system (e.g., tissue culture) to assess the effect of the biomolecule on the system (e.g., cell proliferation or cell death), and the biomolecule may be depleted from a similar system using a particle as described herein to assess the effect of the absence of the biomolecule on the system.

In some aspects, the invention relates to a method for expanding or differentiating a population of cells, comprising contacting a composition comprising the population of cells with a plurality of particles as described herein. The plurality of particles may scavenge one or more molecules that favor an alternate differentiation pathway that competes with a desired differentiation pathway. Thus, the method may favor the differentiation of the population of cells into a desired cell type relative to an alternate cell type. The method may further comprise contacting the composition with a cytokine (e.g., as described herein). The method may further comprise contacting the composition with one or more of a chemokine, interleukin, growth factor, wnt-family protein, tumor necrosis factor, and/or hormone (e.g., as described herein).

The population of cells may comprise stem cells. The population of cells may comprise somatic stem cells or embryonic stem cells. The population of cells may comprise induced stem cells, such as induced pluripotent stem cells. The population of cells may comprise progenitor cells, precursor cells, blast cells, unipotent cells, multipotent stem cells, pluripotent stem cells, and/or intermediate progenitor cells. The population of cells may comprise meiocytes. The population of cells may comprise hematopoietic stem cells, mammary stem cells, intestinal stem cells, mesenchymal stem cells, endothelial stem cells, neural stem cells, olfactory adult stem cells, neural crest stem cells, or testicular cells. The population of cells may comprise satellite cells, oligodendrocytes progenitor cells, thymocytes, angioblasts, bone marrow stromal cells, pancreatic progenitor cells, endothelial progenitor cells, or melanoblasts. The population of cells may comprise multipotential hematopoietic stem cells, common myeloid progenitor cells, myeloblasts, monoblasts, promonocytes, monocytes, common lymphoid progenitor cells, lymphoblasts, prolymphocytes, and/or small lymphocytes.

In some embodiments, the invention relates to a method for differentiating a cell, comprising contacting a composition comprising the cell with a plurality of particles as described herein. The plurality of particles may scavenge one or more molecules that favor an alternate differentiation pathway that competes with a desired differentiation pathway. Thus, the method may favor the differentiation of the cell into a desired cell type relative to an alternate cell type. The method may further comprise contacting the composition with a cytokine (e.g., as described herein). The method may further comprise contacting the composition with one or more of a chemokine, interleukin, growth factor, wnt-family protein, and/or tumor necrosis factor (e.g., as described herein).

The cell may be a stem cell. The cell may be a somatic stem cell or an embryonic stem cell. The cell may be an induced stem cell, such as an induced pluripotent stem cell. The cell may be a progenitor cell, precursor cell, blast cell, unipotent cell, multipotent stem cell, pluripotent stem cell, and/or intermediate progenitor cell. The cell may be a meiocyte. The cell may be a hematopoietic stem cell, mammary stem cell, intestinal stem cell, mesenchymal stem cell, endothelial stem cell, neural stem cell, olfactory adult stem cell, neural crest stem cell, or testicular cell. The cell may be a satellite cell, oligodendrocyte progenitor cell, thymocyte, angioblast, bone marrow stromal cell, pancreatic progenitor cell, endothelial progenitor cell, or melanoblast. The cell may be a multipotential hematopoietic stem cell, common myeloid progenitor cell, myeloblast, monoblast, promonocyte, monocyte, common lymphoid progenitor cell, lymphoblast, prolymphocyte, and/or small lymphocyte.

### XXIV. Kits for Administering the Agent

In certain embodiments, the disclosure also provides a pharmaceutical package or kit comprising one or more containers filled with at least one composition (e.g., particle or particles) of the disclosure. Optionally associated with such container(s) can be a notice in the form prescribed by a governmental agency regulating the manufacture, use or sale of pharmaceuticals or biological products, which notice reflects (a) approval by the agency of manufacture, use or sale for human administration, (b) directions for use, or both.

In certain embodiments, the kit includes additional materials to facilitate delivery of the subject agents. For example, the kit may include one or more of a catheter, tubing, infusion bag, syringe, and the like. In certain embodiments, a composition (e.g., comprising particles as described herein) is packaged in a lyophilized form, and the kit includes at least two containers: a container comprising the lyophilized composition and a container comprising a suitable amount of water, buffer, or other liquid suitable for reconstituting the lyophilized material.

The foregoing applies to any of the compositions and methods described herein. The disclosure specifically contemplates any combination of the features of such compositions and methods (alone or in combination) with the features described for the various kits described in this section.

Unless otherwise defined, all technical and scientific terms used herein have the same meaning as commonly understood by one of ordinary skill in the art to which this disclosure pertains. Preferred methods and materials are described herein, although methods and materials similar or equivalent to those described herein can also be used in the practice or testing of the presently disclosed methods and compositions. All publications, patent applications, patents, and other references mentioned herein are incorporated by reference in their entirety.

The disclosure contemplates all combinations of any of the foregoing aspects and embodiments, as well as combinations with any of the embodiments set forth in the detailed description and examples. These and other aspects of the present disclosure will be further appreciated upon consideration of the following Examples, which are intended to illustrate certain particular embodiments of the disclosure but are not intended to limit its scope, as defined by the claims.

### EXEMPLIFICATION

### Example 1—Method for Treating a Cancer

A human patient is identified by a medical practitioner as having a cancer (e.g., lung, colon, breast, brain, liver, pancreatic, skin, or hematological cancer) that shed soluble TNFR or soluble IL-2R. The patient is administered a composition comprising particles (described herein) that bind to and sequester soluble TNFR or IL-2R in an amount effective to treat the cancer. Optionally, the patient is given “maintenance doses” of the composition to maintain inhibition of the effects of soluble TNFR or IL-2R and thereby continue to enhance immune surveillance against the cancer in the patient.

### Example 2—Method for Detoxifying a Human

A human patient is presents with symptoms of toxicity associated with botulinum toxin. The patient is administered a composition comprising particles (described herein) that bind to and sequester soluble botulinum toxin in an amount effective to ameliorate one or more symptoms associated with the toxicity.

### Example 3—Method for Treating a Viral Infection

A human patient is identified by a medical practitioner as having an HIV-1 infection. The patient is administered a composition comprising particles (described herein) that bind to and sequester soluble HIV-1 virions in an amount effective to reduce titers of the virus in the patient's circulation. The patient is given “maintenance doses” of the composition to maintain reduction of HIV-1 virion titers and thereby suppress the infection in the patient, as well as reduce the likelihood of transmission of the virus to another.

### Example 4—Method for Manufacturing Silicon Particles

Porous silicon disks are manufactured with sizes of 1000 nm by 400 nm and 1000 nm by 800 nm with variable pore sizes. The size and morphology of the disks, as well as pore diameters, are characterized by scanning electron microscopy. Gold nanoparticles (Au) are deposited in the pores of the porous silicon disks. Tumor necrosis factors (TNFs) are conjugated to the surfaces of the gold nanoparticles through dative covalent bonds. The ligand density and TNF-Au binding stabilities are assessed.

### Example 5—Method for Manufacturing Polymer Particles

Poly(lactide-co-glycolide) (PLGA) particles are fabricated by emulsion. The size and morphology of the PLGA particles are characterized by scanning electron microscopy, atomic force microscopy, and transmission electron microscopy. The particles are coated with quaternary ammonium beta-cyclodextrin, for macrophage recruitment (i.e., phagocytosis). The coating is verified by atomic force microscopy and transmission electron microscopy. Coating density and uniformity is characterized by transmission electron microscopy and dynamic light scattering.

The beta-cyclodextrin-coated PLGA particles are incubated with macrophages, and phagocytosis is monitored by fluorescence microscopy and by flow cytometry.

The beta-cyclodextrin-coated PLGA particles are coated with a blend of polyethylene glycol (PEG) and thiol moieties to allow for prevention of opsonization and evasion of macrophage uptake, as well as binding to other particles. The uniformity and density of the PEG and thiol coatings are characterized by atomic force microscopy. Coating stabilities are characterized by incubating the particles in media for various periods of time. Evasion and uptake of the particles are monitored at various time points by incubating the particles with macrophages, as described above.

The PLGA particles are coated with tumor necrosis factor (TNF), and the particles are combined by disulfide bonds to form a “sponge,” comprising TNF on the interior surface of the sponge. The exterior surface (i.e., outer surface) of the sponge is optionally blocked with particles that do not comprise TNF to prevent interactions between the TNF of the sponge and cells.

### Example 6—Pharmacokinetics of Polymer-Based Particles

The sponge of Example 5 (i.e., a composition comprising “sponges” of Example 5, such as 10³ to 10¹² sponges) is administered either intravenously or intratumorally into mouse models of primary and metastatic cancer as well as healthy controls. The toxicity of the sponge is determined by identifying LD₅₀'s for each route of administration. The half-life of the sponge is determined by monitoring plasma concentrations of the sponge by LC/MS and ICP for each route of administration. The biodistribution of the sponge is determined by taking biopsies of the mice and analyzing tissue for the sponge and its components by LC/MS, ICP, and confocal microscopy.

### Example 7—Efficacy of Polymer-based Particles

The sponge of Example 5 (i.e., a composition comprising “sponges” of Example 5, such as 10³ to 10¹² sponges) is administered to mice comprising MDA-MB-231 or 4T1 xenographs. The MDA-MB-231 model is used to assess reductions in tumor size and growth, and the 4T1 model is used to assess inhibition of metastasis. The sponge is administered intratumorally to MDA-MB-231 mice once a week for 6 weeks, and body weight and tumor sizes are monitored periodically. The sponge is administered intravenously to 4T1 mice once a week for 6 weeks, and the number of metastases are monitored.

### Example 8—Pharmacokinetics and Efficacy of Silicon/Gold-based Particles

The experiments of Examples 6 and 7 are repeated with the porous silicon particles of Example 5.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

What is claimed is:

1. A particle having at least one surface and an agent immobilized on the surface, wherein:
the agent selectively binds to a target that is a first member of a specific binding pair; and
binding of the target to the particle inhibits the interaction of the target with a second member of the specific binding pair.

2. A particle, comprising a surface and an agent immobilized on the surface, wherein:
the agent can selectively bind to a target; and
binding of an agent to the target inhibits interactions between the target and a cell.

3. The particle of claim 1 or 2, wherein the particle is shaped and sized to circulate in the vasculature of a subject.

4. The particle of any one of the preceding claims, wherein the particle is larger than 1 μm.

5. The particle of any one of the preceding claims, wherein the longest dimension of the particle is no greater than about 5 μm.

6. The particle of any one of the preceding claims, wherein the smallest dimension of the particle is at least about 300 nm.

7. The particle of any one of the preceding claims, further comprising a plurality of coating molecules.

8. The particle of claim 7, wherein:
the particle comprises an interior surface and an exterior surface;
the agent is immobilized on the interior surface and the exterior surface;
the plurality of coating molecules are bound to the exterior surface; and
the coating molecules inhibit interactions between the agent and molecules on a cell surface.

9. The particle of claim 7 or 8, wherein the plurality of coating molecules increases the clearance of the particle in vivo.

10. The particle of claim 9, wherein the plurality of coating molecules increase the clearance of the particle by phagocytosis, renal clearance, or hepatobiliary clearance.

11. The particle of claim 7 or 8, wherein the plurality of coating molecules decreases the clearance of the particle in vivo.

12. The particle of claim 7 or 8, wherein the plurality of coating molecules inhibits interactions between the agent and either cells or extracellular proteins.

13. The particle of any one of claims 7 to 12, wherein the plurality of coating molecules comprises a polymer.

14. The particle of any one of claims 7 to 13, wherein the plurality of coating molecules is biodegradable.

15. The particle of any one of the preceding claims, wherein the particle is dendritic.

16. The particle of any one of the preceding claims, wherein:
the particle is porous;
the surface comprises outer surfaces and inner surfaces; and
the inner surfaces consist of the inner walls of the pores of the particle.

17. The particle of claim 16, wherein the agent is immobilized on the inner surfaces.

18. The particle of claim 16 or 17, wherein a plurality of pores have a cross-sectional dimension of at least 50 nm.

19. The particle of any one of claims 16 to 18, wherein the particle has a porosity of about 40% to about 95%.

20. The particle of any one of claims 16 to 19, wherein the particle comprises metal, gold, alumina, glass, silica, silicon, starch, agarose, latex, plastic, polyacrylamide, methacrylate, a polymer, or a nucleic acid.

21. The particle of claim 20, wherein the particle comprises porous silicon.

22. The particle of any one of the preceding claims, wherein the particle is substantially cubic, pyramidal, conic, spherical, tetrahedral, hexahedral, octahedral, dodecahedral, or icosahedral.

23. The particle of any one of the preceding claims, wherein the particle comprises one or more outward-facing protrusions.

24. The particle of claim 23, wherein the particle comprises more than one outward-facing protrusion.

25. The particle of any one of the preceding claims, wherein the particle comprises:
one or more vertices; and
one or more outward-facing protrusions pointing outward from at least one of its vertices.

26. The particle of any one of claims 23 to 25, wherein one or more protrusions are sized and oriented to inhibit: (i) the agent immobilized on the surface of the particle from binding or activating a cell surface receptor protein and/or (ii) when the target is bound to the agent, the interaction of the target and a second member of a specific binding pair of which the target is the first member.

27. The particle of any one of the preceding claims, wherein the particle comprises two intersecting ridges extending from the surface of the particle, and the ridges are sized and oriented to inhibit: (i) the agent immobilized on the surface of the particle from binding or activating a cell surface receptor protein and/or (ii) when the target is bound to the agent, the interaction of the target and a second member of a specific binding pair of which the target is the first member.

28. The particle of any one of the preceding claims, wherein the particle comprise a tube.

29. The particle of claim 28, wherein the agent is immobilized on the inner surface of the tube.

30. The particle of claim 28 or 29, wherein the tube comprises at least one open end.

31. The particle of any one of claims 28 to 30, wherein the tube is a cylindrical tube, triangular tube, square tube, pentagonal tube, hexagonal tube, heptagonal tube, octahedral tube, or an irregularly-shaped tube.

32. The particle of any one of claims 28 to 31, wherein the particle comprises more than one tube.

33. The particle of claim 32, wherein the particle comprises a lattice defined by a plurality of tubes.

34. The particle of any one of claims 28 to 33, wherein the tube comprises a protein, nucleic acid, or polymer.

35. The particle of any one of claims 1 to 22, wherein:
the particle comprises a core subparticle and a plurality of protecting subparticles; and
the agent is immobilized on the core subparticle.

36. The particle of claim 35, wherein the core subparticle is about 100 nm to about 2 μm in size.

37. The particle of claim 35 or claim 36, wherein the protecting subparticles are about 10 nm to about 1 μm in size.

38. The particle of any one of claims 35 to 37, wherein the particle comprises 4 to 10⁶ protecting subparticles.

39. The particle of any one of claims 35 to 38, wherein the particle comprises more than one core subparticle.

40. The particle of any one of claims 1 to 14, wherein the particle is a 2-dimensional shape.

41. The particle of claim 40, wherein the shape is a circle, ring, cross, fishbone, ellipse, triangle, square, pentagon, hexagon, heptagon, octagon, or star.

42. The particle of any one of the preceding claims, wherein the agent is oriented on the particle such that it has a reduced ability to bind to a molecule on the surface of a cell.

43. The particle of claim 42, wherein the agent is oriented on the particle such that it has a reduced ability to bind to a target on the surface of a cell.

44. The particle of any one of the preceding claims, wherein the agent is oriented on the particle such that it is sterically inhibited from binding to a molecule on the surface of a cell.

45. The particle of claim 44, wherein the agent is oriented on the particle such that it is sterically inhibited from binding to a target on the surface of a cell.

46. The particle of any one of the preceding claims, wherein the surface is oriented such that the agent has a reduced ability to bind to a molecule on the surface of a cell.

47. The particle of any one of the preceding claims, wherein the agent has a reduced ability to activate a cell surface receptor protein, relative to the ability of a natural ligand of the cell surface receptor protein.

48. The particle of claim 47, wherein the agent does not activate the cell surface receptor protein.

49. The particle of any one of claims 1 to 48, wherein the particle comprises void space.

50. The particle of any one of claims 1 to 49, wherein the isoelectric point of the particle is about 5 to about 9.

51. The particle of any one of claims 1 to 50, wherein the target is a viral protein.

52. The particle of claim 51, wherein the viral protein is from arbovirus, adenovirus, alphavirus, arenaviruses, astrovirus, BK virus, bunyaviruses, calicivirus, cercopithecine herpes virus 1, Colorado tick fever virus, coronavirus, Coxsackie virus, Crimean-Congo hemorrhagic fever virus, cytomegalovirus, Dengue virus, ebola virus, echinovirus, echovirus, enterovirus, Epstein-Barr virus, flavivirus, foot-and-mouth disease virus, hantavirus, hepatitis A, hepatitis B, hepatitis C, herpes simplex virus I, herpes simplex virus II, human herpes virus, human immunodeficiency virus type I (HIV-I), human immunodeficiency virus type II (HIV-II),human papillomavirus, human T-cell leukemia virus type I, human T-cell leukemia virus type II, influenza, Japanese encephalitis, JC virus, Junin virus, lentivirus, Machupo virus, Marburg virus, measles virus, mumps virus, naples virus, norovirus, Norwalk virus, orbiviruses, orthomyxovirus, papillomavirus, papovavirus, parainfluenza virus, paramyxovirus, parvovirus, picornaviridae, poliovirus, polyomavirus, poxvirus, rabies virus, reovirus, respiratory syncytial virus, rhinovirus, rotavirus, rubella virus, sapovirus, smallpox, togaviruses, Toscana virus, varicella zoster virus, West Nile virus, or Yellow Fever virus.

53. The particle of claim 51 or 52, wherein the viral protein is a viral capsid protein or a viral envelope protein.

54. The particle of any one or claims 1 to 50, wherein the target is a bacterial protein or a component of a bacterial cell wall.

55. The particle of claim 54, wherein the bacterial protein or cell wall component is from Actinomyces israelii, Bacillus anthracis, Bacillus cereus, Bacteroides fragilis, Bartonella henselae, Bartonella Quintana, Bordetella pertussis, Borrelia burgdorferi, Borrelia garinii, Borrelia afzelii, Borrelia recurrentis, Brucella abortus, Brucella canis, Brucella melitensis, Brucella suis, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium diptheriae, Ehrlichia canis, Ehrlichia chaffeensis, Enterococcus faecalis, Enterococcus faecium, Escherichia coli, Francisella tularensis, Haemophilus influenzae, Haemophilus vaginalis, Helicobacter pylori, Klebsiella pneumoniae, Legionella pneumophila, Leptospira interrogans, Leptospira santarosai, Leptospira weilii, Leptospira noguchii, Listeria monocytogenes, Mycobacterium leprae, Mycobacterium tuberculosis, Mycobacterium ulcerans, Mycoplasma pneumoniae, Neisseria gonorrhoeae, Neisseria meningitidis, Pseudomonas aeruginosa, Nocardia asteroides, Rickettsia rickettsii, Salmonella typhi, Salmonella typhimurium, Shigella sonnei, Shigella dysenteriae, Staphylococcus aureus, Staphylococcus epidermidis, Staphylococcus saprophyticus, Streptococcus agalactiae, Streptococcus pneumoniae, Streptococcus pyogenes, Streptococcus viridans, Treponema pallidum, Ureaplasma urealyticum, Vibrio cholerae, Yersinia pestis, Yersinia enterocolitica, or Yersinia pseudotuberculosis.

56. The particle of any one of claims 1 to 50, wherein the target is a yeast or fungal protein or a component of a yeast or fungal cell wall.

57. The particle of claim 56, wherein the yeast or fungal protein or cell wall component is from Apophysomyces variabilis, Aspergillus clavatus, Aspergillus flavus, Aspergillus fumigatus, Basidiobolus ranarum, Candida albicans, Candida glabrata, Candida guilliermondii, Candida krusei, Candida lusitaniae, Candida parapsilosis, Candida tropicalis, Candida stellatoidea, Candida viswanathii, Conidiobolus coronatus, Conidiobolus incongruous, Cryptococcus albidus, Cryptococcus gattii, Cryptococcus laurentii, Cryptococcus neoformans, Encephalitozoon intestinalis, Enterocytozoon bieneusi, Exophiala jeanselmei, Fonsecaea compacta, Fonsecaea pedrosoi, Geotrichum candidum, Histoplasma capsulatum, Lichtheimia corymbifera, Mucor indicus, Paracoccidioides brasiliensis, Phialophora verrucosa, Pneumocystis carinii, Pneumocystis jirovecii, Pseudallescheria boydii, Rhinosporidium seeberi, Rhodotorula mucilaginosa, Stachybotrys chartarum, Syncephalastrum racemosum, or Rhizopus oryzae.

58. The particle of any one of claims 1 to 50, wherein the target is a protozoan protein.

59. The particle of claim 58, wherein the protozoan protein is from Cryptosporidium, Giardia intestinalis, Giardia lamblia, Leishmania aethiopica, Leishmania braziliensis, Leishmania donovani, Leishmania infantum, Leishmania major, Leishmania mexicana, Leishmania tropica, Plasmodium coatneyi, Plasmodium falciparum, Plasmodium garnhami, Plasmodium inui, Plasmodium odocoilei, Trichomonas gallinae, Trichomonas vaginalis, Tritrichomonas foetus, Trypanosoma brucei, Trypanosoma cruzi, Trypanosoma equiperdum, Trypanosoma evansi, Trypanosoma lewisi, Trypanosoma pestanai, Trypanosoma suis, or Trypanosoma vivax,

60. The particle of any one of claims 1 to 50, wherein the target is a toxin.

61. The particle of claim 60, wherein the toxin is a bacterial toxin, a plant toxin, or a zootoxin.

62. The particle of claim 60 or 61, wherein the toxin is melittin, brevetoxin, tetrodotoxin, chlorotoxin, tetanus toxin, bungarotoxin, Clostridium botulinum toxin, ricin, epsilon toxin of Clostridium perfringens, Staphylococcus enterotoxin B, or endotoxin.

63. The particle of any one of claims 1 to 50, wherein the target is a poison, venom, allergen, carcinogen, psychoactive drug, or an agent of a chemical weapon.

64. The particle of any one of claims 1 to 50, wherein the target is selected from TNFα, TNFβ, a soluble TNF receptor, soluble TNFR-1, soluble TNFR-2, lymphotoxin, lymphotoxin alpha, lymphotoxin beta, 4-1BB Ligand, CD30 Ligand, EDA-A1, LIGHT, TL1A, TWEAK, TRAIL, soluble TRAIL receptor, IL-1, soluble IL-1 receptor, IL-1A, soluble IL-1A receptor, IL-1B, soluble IL-1B receptor, IL-2, soluble IL-2 receptor, IL-5, soluble IL-5 receptor, IL-6, soluble IL-6 receptor, IL-8, IL-10, soluble IL-10 receptor, CXCL1, CXCL8, CXCL9, CXCL10, CX3CL1, FAS ligand, soluble death receptor-3, soluble death receptor-4, soluble death receptor-5, TNF-related weak inducer of apoptosis, MMP1, MMP2, MMP3, MMP9, MMP10, MMP12, CD28, a soluble member of the B7 family, soluble CD80/B7-1, soluble CD86/B7-2, soluble CTLA4, soluble PD-L1, soluble PD-1, soluble Tim3, Tim3L, galectin 3, galectin 9, soluble CEACAM1, soluble LAG3, TGF-β, TGF-β1, TGF-β2, TGF-β3, anti-mullerian hormone, artemin, glial cell-derived neurotrophic factor, a bone morphogenic protein (e.g., BMP2, BMP3, BMP3B, BMP4, BMP5, BMP6, BMP7, BMP8A, BMP8B, BMP10, BMP 11, BMP 12, BMP13, BMP15), a growth differentiation factor (e.g., GDF1, GDF2, GDF3, GDF3A, GDF5, GDF6, GDF7, GDF8, GDF9, GDF10, GDF11, GDF15), inhibin alpha, inhibin beta (e.g., inhibin beta A, B, C, E), lefty, nodal, neurturin, persephin, myostatin, ghrelin, sLR11, CCL2, CCL5, CCL11, CCL12, CCL19, interferon alpha, interferon beta, interferon gamma, clusterin, VEGF-A, granulocyte colony-stimulating factor (G-CSF), granulocyte-macrophage colony-stimulating factor (GM-CSF), prostaglandin E2, hepatocyte growth factor, nerve growth factor, sclerostin, complement C5, angiopoietin 2, angiopoietin 3, PCSK9, amyloid beta, activin, activin A, activin B, β2 microglobulin, soluble NOTCH1, soluble NOTCH2, soluble NOTCH3, soluble NOTCH4, haptoglobin, fibrinogen alpha chain, corticotropin releasing factor, corticotropin releasing factor type 1, corticotropin releasing factor type 2, urocortin 1, urocortin 2, urocortin 3, CD47, an anti-interferon γ autoantibody, an anti-interleukin 6 autoantibody, an anti-interleukin 17 autoantibody, an anti-ghrelin autoantibody, wnt, indoleamine 2,3-dioxygenase, C-reactive protein, and HIV-1 gp120.

65. The particle of any one of claims 1 to 50 and 64, wherein the agent comprises an antibody, or an antigen-binding portion thereof, which specifically binds to TNFα, TNFβ, a soluble TNF receptor, soluble TNFR-1, soluble TNFR-2, lymphotoxin, lymphotoxin alpha, lymphotoxin beta, 4-1BB Ligand, CD30 Ligand, EDA-A1, LIGHT, TL1A, TWEAK, TRAIL, soluble TRAIL receptor, IL-1, soluble IL-1 receptor, IL-1A, soluble IL-1A receptor, IL-1B, soluble IL-1B receptor, IL-2, soluble IL-2 receptor, IL-5, soluble IL-5 receptor, IL-6, soluble IL-6 receptor, IL-8, IL-10, soluble IL-10 receptor, CXCL1, CXCL8, CXCL9, CXCL10, CX3CL1, FAS ligand, soluble death receptor-3, soluble death receptor-4, soluble death receptor-5, TNF-related weak inducer of apoptosis, MMP1, MMP2, MMP3, MMP9, MMP10, MMP12, CD28, a soluble member of the B7 family, soluble CD80/B7-1, soluble CD86/B7-2, soluble CTLA4, soluble PD-L1, soluble PD-1, soluble Tim3, Tim3L, galectin 3, galectin 9, soluble CEACAM1, soluble LAG3, TGF-β, TGF-β1, TGF-β2, TGF-β3, anti-mullerian hormone, artemin, glial cell-derived neurotrophic factor, a bone morphogenic protein (e.g., BMP2, BMP3, BMP3B, BMP4, BMP5, BMP6, BMP7, BMP8A, BMP8B, BMP10, BMP 11, BMP 12, BMP13, BMP15), a growth differentiation factor (e.g., GDF1, GDF2, GDF3, GDF3A, GDF5, GDF6, GDF7, GDF8, GDF9, GDF10, GDF11, GDF15), inhibin alpha, inhibin beta (e.g., inhibin beta A, B, C, E), lefty, nodal, neurturin, persephin, myostatin, ghrelin, sLR11, CCL2, CCL5, CCL11, CCL12, CCL19, interferon alpha, interferon beta, interferon gamma, clusterin, VEGF-A, granulocyte colony-stimulating factor (G-CSF), granulocyte-macrophage colony-stimulating factor (GM-CSF), prostaglandin E2, hepatocyte growth factor, nerve growth factor, sclerostin, complement C5, angiopoietin 2, angiopoietin 3, PCSK9, amyloid beta, activin, activin A, activin B, β2 microglobulin, soluble NOTCH1, soluble NOTCH2, soluble NOTCH3, soluble NOTCH4, haptoglobin, fibrinogen alpha chain, corticotropin releasing factor, corticotropin releasing factor type 1, corticotropin releasing factor type 2, urocortin 1, urocortin 2, urocortin 3, CD47, an anti-interferon γ autoantibody, an anti-interleukin 6 autoantibody, an anti-interleukin 17 autoantibody, an anti-ghrelin autoantibody, wnt, indoleamine 2,3-dioxygenase, C-reactive protein, or HIV-1 gp120.

66. The particle of any one of claims 1 to 50 and 64, wherein the agent comprises TNFα, TNFβ, a soluble TNF receptor, soluble TNFR-1, soluble TNFR-2, vTNF, lymphotoxin, lymphotoxin alpha, lymphotoxin beta, 4-1BB Ligand, CD30 Ligand, EDA-A1, LIGHT, TL1A, TWEAK, TRAIL, soluble TRAIL receptor, IL-1, soluble IL-1 receptor, IL-1A, soluble IL-1A receptor, IL-1B, soluble IL-1B receptor, IL-2, soluble IL-2 receptor, IL-5, soluble IL-5 receptor, IL-6, soluble IL-6 receptor, IL-8, IL-10, soluble IL-10 receptor, CXCL1, CXCL8, CXCL9, CXCL10, CX3CL1, FAS ligand, soluble death receptor-3, soluble death receptor-4, soluble death receptor-5, TNF-related weak inducer of apoptosis, MMP1, MMP2, MMP3, MMP9, MMP10, MMP12, CD28, a soluble member of the B7 family, soluble CD80/B7-1, soluble CD86/B7-2, soluble CTLA4, soluble PD-L1, soluble PD-1, soluble Tim3, Tim3L, galectin 3, galectin 9, soluble CEACAM1, soluble LAG3, TGF-β, TGF-β1, TGF-β2, TGF-β3, sLR11, CCL2, CCL5, CCL11, CCL12, CCL19, activin, activin A, activin B, soluble NOTCH1, soluble NOTCH2, soluble NOTCH3, soluble NOTCH4, soluble Jagged1, soluble Jagged2, soluble DLL1, soluble DLL3, soluble DLL4, or haptoglobin.

67. The particle of any one of claims 1 to 50, 64, and 65, wherein the agent comprises ipilimumab, pembrolizumab, nivolumab, infliximab, adalimumab, certolizumab, golimumab, etanercept, stamulumab, fresolimumab, metelimumab, demcizumab, tarextumab, brontictuzumab, mepolizumab, urelumab, canakinumab, daclizumab, belimumab, denosumab, eculizumab, tocilizumab, atlizumab, ustekinumab, palivizumab, bevacizumab, brolucizumab, ranibizumab, aflibercept, actoxumab, elsilimomab, siltuximab, afelimomab, nerelimomab, ozoralizumab, pateclizumab, sirukumab, omalizumab, aducanumab, bapineuzumab, crenezumab, gantenerumab, ponezumab, solanezumab, dapirolizumab, ruplizumab, toralizumab, enoticumab, alacizumab, cetuximab, futuximab, icrucumab, imgatuzumab, matuzumab, necitumumab, nimotuzumab, panitumumab, ramucirumab, zalutumumab, duligotumab, patritumab, ertumaxomab, pertuzumab, trastuzumab, alirocumab, anrukinzumab, diridavumab, drozitumab, dupilumab, dusigitumab, eculizumab, edobacomab, efungumab, eldelumab, enoblituzumab, enokizumab, evinacumab, evolocumab, exbivirumab, exbivirumab, fasinumab, felvizumab, fezakinumab, ficlatuzumab, firivumab, fletikumab, foralumab, foravirumab, fulranumab, faliximab, ganitumab, gevokizumab, fuselkumab, idarucizumab, imalumab, inolimomab, iratumumab, ixekizumab, lampalizumab, lebrikizumab, lenzilumab, lerdelimumab, lexatumumab, libivirumab, ligelizumab, lodelcizumab, lulizumab, mapatumumab, motavizumab, namilumab, nebacumab, nesvacumab, obiltoxaximab, olokizumab, orticumab, pagibaximab, palivizumab, panobacumab, pascolizumab, perakizumab, pidilizumab, pexelizumab, pritoxaximab, quilizumab, radretumab, rafivirumab, ralpancizumab, raxibacumab, regavirumab, reslizumab, rilotumumab, romosozumab, rontalizumab, sarilumab, secukinumab, setoxaximab, sevirumab, sifalimumab, siltuximab, suvizumab, tabalumab, tacatuzumab, talizumab, tanezumab, tefibazumab, TGN1412, tildrakizumab, tigatuzumab, TNX-650, tosatoxumab, tralokinumab, tremelimumab, trevogrumab, tuvirumab, urtoxazumab, vantictumab, vanucizumab, or an antigen-binding portion of any one of the foregoing.

68. The particle of any one of the preceding claims, wherein the target is a soluble biomolecule.

69. The particle of any one of the preceding claims, wherein the target is:
a target as described anywhere herein, supra;
a biomolecule as described anywhere herein, supra;
a soluble biomolecule as described anywhere herein, supra; or
an antigen of an antibody as described anywhere herein, supra.

70. The particle of any one of the preceding claims, wherein:
the agent is an agent as described anywhere herein, supra;
the agent comprises an antibody, wherein the antibody is described anywhere herein, supra;
the agent comprises an antigen-binding portion of an antibody, wherein the antibody is described anywhere herein, supra; or
the agent comprises an antibody, or an antigen-binding portion thereof, that specifically binds to a target, biomolecule, or soluble biomolecule, wherein the target, biomolecule, or soluble biomolecule is described anywhere herein, supra.

71. The particle of any one of the preceding claims, wherein the longest dimension of the particle is no greater than about 1 μm.

72. The particle of any one of the preceding claims, wherein:
the target is a soluble biomolecule;
the soluble biomolecule is a form of a cell surface receptor protein; and
the agent is oriented on the particle such that it is sterically inhibited from binding or activating the cell surface receptor protein on the surface of a cell.

73. A particle having at least one surface and an agent immobilized on the surface, wherein:
the agent selectively binds to a soluble biomolecule;
the soluble biomolecule is a form of a cell surface receptor protein; and
the agent is oriented on the particle such that the agent is sterically inhibited from binding or activating the cell surface receptor protein on the surface of a cell.

74. The particle of any one of the preceding claims, wherein the agent is a ligand of a cell surface receptor protein.

75. The particle of claim 74, wherein the agent is a natural ligand of the cell surface receptor protein.

76. The particle of any one of claims 72 to 75, wherein the cell surface receptor protein is expressed by a cancer cell.

77. The particle of any one of claims 72 to 76, wherein the cell surface receptor protein is a protein shed by a cancer cell as a soluble form of the cell surface receptor protein.

78. The particle of any one of claims 72 to 77, wherein the cell surface receptor protein, when activated on a cell surface, induces apoptosis.

79. The particle of any one of claims 72 to 78, wherein the cell surface receptor protein is a tumor necrosis factor receptor (TNFR) protein.

80. The particle of any one of claims 72 to 78, wherein the cell surface receptor protein is a Fas receptor protein.

81. The particle of any one of claims 72 to 78, wherein the cell surface receptor protein is a TNF-related apoptosis-inducing ligand receptor (TRAILR) protein, 4-1BB receptor protein, CD30 protein, EDA receptor protein, HVEM protein, lymphotoxin beta receptor protein, DR3 protein, or TWEAK receptor protein.

82. The particle of any one of claims 72 to 81, wherein the agent comprises a tumor necrosis factor (TNF) family ligand or a variant thereof.

83. The particle of claim 82, wherein the TNF family ligand is TNFα.

84. The particle of claim 82, wherein the TNF family ligand is selected from Fas ligand, lymphotoxin, lymphotoxin alpha, lymphotoxin beta, 4-1BB Ligand, CD30 Ligand, EDA-A1, LIGHT, TL1A, TWEAK, TNFβ, and TRAIL.

85. The particle of any one of claims 72 to 78, wherein the cell surface receptor protein is an interleukin receptor protein.

86. The particle of claim 85, wherein the interleukin receptor protein is an IL-2 receptor protein.

87. The particle of claim 85 or 86, wherein the agent is an interleukin protein or variant thereof.

88. The particle of claim 87, wherein the interleukin protein is an IL-2 protein.

89. A plurality of particles according to any one of the preceding claims.

90. The plurality of particles of claim 89, wherein the mean particle size is greater than 1 μm.

91. The plurality of particles of claim 89, wherein the mean particle size is 1 μm to 5 μm.

92. A method for treating a subject afflicted with a cancer, comprising administering to the subject the plurality of particles of any one of claims 89 to 91, wherein:
the cancer comprises cells that shed a soluble form of at least one cell surface receptor protein; and
the plurality of particles inhibits the biological activity of the shed soluble form of the at least one cell surface receptor protein, thereby treating the cancer.

93. The method of claim 92, wherein the cancer cells shed a soluble form of TNF receptor.

94. The method of claim 93, wherein each particle of the plurality comprises an agent comprising a TNFα polypeptide or a variant thereof.

95. The method of claim 92, wherein the cancer cells shed a soluble form of IL-2 receptor.

96. The method of claim 95, wherein each particle of the plurality comprises an agent comprising a IL-2 polypeptide or a variant thereof.

97. The method of any one of claims 92 to 96, wherein the subject has received adoptive cell transfer therapy (ACT).

98. The method of any one of claims 92 to 97, further comprising administering adoptive cell transfer therapy to the subject.

99. The method of claim 97 or 98, wherein the adoptive cell transfer therapy is the administration of a composition comprising lymphocytes to the subject.

100. The method of claim 99, wherein the lymphocytes are tumor-infiltrating lymphocytes (TILs).

101. The method of claim 99 or 100, wherein the lymphocytes comprise a chimeric antigen receptor (CAR).

102. A method for treating a subject afflicted with an autoimmune disease, comprising administering to the subject a plurality of particles of any one of claims 89 to 91.

103. The method of claim 102, wherein the target is interleukin 1A, interleukin 1B, interleukin 2, interleukin 5, interleukin 6, interleukin 8, tumor necrosis factor alpha, fas ligand, TNF-related apoptosis inducing ligand, CXCL8, CXCL1, CD80/B7-1, CD86/B7-2, or PD-L1.

104. A method for treating a subject afflicted with a neurodegenerative disease, comprising administering to the subject a plurality of particles of any one of claims 89 to 91.

105. The method of claim 104, wherein the target is amyloid (3.

106. A method of promoting healthy aging in a subject, comprising administering to the subject a plurality of particles of any one of claims 89 to 91.

107. The method of claim 106, wherein the target is TGF-β1, CCL11, MCP-1/CCL2, beta-2 microglobulin, GDF-8/myostatin, or haptoglobin.

108. A method for treating a metabolic disorder in a subject, comprising administering to the subject a plurality of particles of any one of claims 89 to 91.

109. The method of claim 108, wherein the target is ghrelin, an anti-ghrelin autoantibody, or cortisol.

110. A method for increasing muscle mass in a subject, comprising administering to the subject a plurality of particles of any one of claims 89 to 91.

111. The method of claim 110, wherein the target is myostatin or TGF-β1.

112. The method of any one of claims 92 to 111, wherein the subject is a mammal.

113. The method of claim 112, wherein the subject is a human.
